SEQUENCE LISTING (12 sequences)

SEQ ID NO: 1 - DNA, 29882 bp, SARS-CoV-2
  source 1..29882: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaa

SEQ ID NO: 2 - protein, 1273 aa, synthetic construct
  REGION 1..1273: note = Recombinant protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 22347 bp, synthetic construct
  misc_feature 1..22347: note = recombinant polynucleotide
  source 1..22347: mol_type = other DNA
accaaacaagagaagaaacttgtctgggaatataaatttaactttaaattaacttaggattaaagacattgactagaaggtcaagaaaagggaactctataatttcaaaaatgttgagcctatttgatacatttaatgcacgtaggcaagaaaacataacaaaatcagccggtggagctatcattcctggacagaaaaatactgtctctatattcgcccttggaccgacaataactgatgataatgagaaaatgacattagctcttctatttctatctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaagatctaaaacggcaaaagtatggaggatttgtggttaagacgagagagatgatatatgaaaagacaactgattggatatttggaagtgacctggattatgatcaggaaactatgttgcagaacggcaggaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagaccagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgccgaagctcaaatgagctcaacactggaagatgaacttggagtgacacacgaatctaaagaaagcttgaagagacatataaggaacataaacagttcagagacatctttccacaaaccgacaggtggatcagccatagagatggcaatagatgaagagccagaacaattcgaacatagagcagatcaagaacaaaatggagaacctcaatcatccataattcaatatgcctgggcagaaggaaatagaagcgatgatcagactgagcaagctacagaatctgacaatatcaagaccgaacaacaaaacatcagagacagactaaacaagagactcaacgacaagaagaaacaaagcagtcaaccacccactaatcccacaaacagaacaaaccaggacgaaatagatgatctgtttaacgcatttggaagcaactaatcgaatcaacattttaatctaaatcaataataaataagaaaaacttaggattaaagaatcctatcataccggaatatagggtggtaaatttagagtctgcttgaaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacttatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctggaggaatccccagaagcatcacagattctaaaaatggaacccaaaacacggaggatattgatctcaatgaaattagaaagatggataaggactctattgaggggaaaatgcgacaatctgcaaatgttccaagcgagatatcaggaagtgatgacatatttacaacagaacaaagtagaaacagtgatcatggaagaagcctggaatctatcagtacacctgatacaagatcaataagtgttgttactgctgcaacaccagatgatgaagaagaaatactaatgaaaaatagtaggacaaagaaaagttcttcaacacatcaagaagatgacaaaagaattaaaaaagggggaaaagggaaagactggtttaagaaatcaaaagataccgacaaccagataccaacatcagactacagatccacatcaaaagggcagaagaaaatctcaaagacaacaaccaccaacaccgacacaaaggggcaaacagaaatacagacagaatcatcagaaacacaatcctcatcatggaatctcatcatcgacaacaacaccgaccggaacgaacagacaagcacaactcctccaacaacaacttccagatcaacttatacaaaagaatcgatccgaacaaactctgaatccaaacccaagacacaaaagacaaatggaaaggaaaggaaggatacagaagagagcaatcgatttacagagagggcaattactctattgcagaatcttggtgtaattcaatccacatcaaaactagatttatatcaagacaaacgagttgtatgtgtagcaaatgtactaaacaatgtagatactgcatcaaagatagatttcctggcaggattagtcataggggtttcaatggacaacgacacaaaattaacacagatacaaaatgaaatgctaaacctcaaagcagatctaaagaaaatggacgaatcacatagaagattgatagaaaatcaaagagaacaactgtcattgatcacgtcactaatttcaaatctcaaaattatgactgagagaggaggaaagaaagaccaaaatgaatccaatgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatattaagttcatacaatgagtcaaatgcaacaagactaatacccaaaaaagtgagcagtacaatgagatcactagttgcagtcatcaacaacagcaatctctcacaaagcacaaaacaatcatacataaacgaactcaaacgttgcaaaaatgatgaagaagtatctgaattaatggacatgttcaatgaagatgtcaacaattgccaatgatccaacaaagaaacgacaccgaacaaacagacaagaaacaacagtagatcaaaacctgtcaacacacacaaaatcaagcagaatgaaacaacagatacttaagaatatacaaataagaaaaacttaggattaaagagccatggtcgtgttcctagtcctactaccgctagtctcttctcagtgtgtaaacctaacaacgagaacacaactaccaccggcgtacaccaattctttcacaagaggagtatattacccggacaaggtgttcagatcctccgtactacattctacacaggacctattcctaccgttcttctctaacgtaacatggttccacgcgatccatgtctctggaacaaacggaacgaagagattcgataacccggtcttgccgttcaacgatggtgtatactttgcgtccaccgagaagtccaacatcatcagaggatggatcttcggaaccaccttggattctaagacccagtccttgctaatcgtcaacaacgcgaccaacgtcgtcatcaaagtctgcgaattccagttctgtaacgacccgttcttgggagtctactaccacaagaacaacaagtcctggatggaatccgagttcagagtctactcttccgcgaacaactgcaccttcgaatatgtatctcagccgttcctaatggacctagagggaaagcagggaaacttcaagaacctaagagagttcgtattcaagaacatcgacggatacttcaagatctactccaagcacactccgatcaacctagttagagatctaccgcaaggattctctgcgctagaaccgttagtagatttgccgatcggaatcaacatcaccagattccagacactactagcgctacacagatcttacctaacgccgggagattcttcttctggatggactgctggtgctgcggcttattatgtaggatacctacagccgagaaccttcctattgaagtacaacgaaaacggaaccatcaccgatgccgtagattgtgctctagatccgctatccgaaacgaagtgcaccctaaagtctttcaccgtcgagaagggaatctaccagacctccaactttagagtacagccgaccgaatccatcgtcagatttccgaacatcacgaacctatgtccgttcggagaagtgttcaacgcgacaagatttgcgtctgtctatgcgtggaacagaaaaagaatcagtaactgcgtcgcggactactccgtcctatacaactctgcctctttctccacgttcaaatgctacggtgtatctccgacaaagctaaacgatctatgcttcaccaacgtctacgcggactccttcgtaatcagaggagatgaagttagacagattgcgccgggacaaactggaaagatcgcggattataactacaagctaccggacgacttcaccggatgtgtaattgcgtggaattcgaacaacctagactccaaagtcggaggaaactacaactacttgtacagactattcagaaagtccaacctaaagccgttcgagagagacatctccaccgaaatctatcaggctggatctacaccgtgtaatggtgtcgaaggattcaactgctacttcccgctacagtcttacggatttcaaccgacaaacggtgtaggatatcagccgtacagagtcgtcgtactatccttcgaactactacatgctccggcgacagtatgtggaccgaaaaagtctaccaacctagtcaagaacaaatgcgtcaactttaacttcaacggactaaccggaaccggtgtcctaaccgaatctaacaagaagtttctaccgttccagcagttcggaagagatatcgcggatacaacagacgctgtcagagatccgcaaaccttggagatcctagatatcacaccgtgttctttcggtggtgtctctgtaattactccgggaacgaacacctccaatcaagtagcggtactataccaggacgtgaactgtacagaagtaccggtagctattcacgcggatcaactaacaccaacttggagagtgtactccaccggatctaacgtattccaaacaagagcgggatgtctaatcggagcggaacacgtaaacaactcctacgaatgtgatatcccgattggagcgggaatctgtgcgtcttaccaaacacaaacaaactctccgagaagagcgagatctgtagcctctcaatctattatcgcctacaccatgtccttgggagccgaaaattctgtcgcgtactccaacaattctatcgcgatcccgacaaacttcaccatctctgtaacaaccgagatcctaccggtgtctatgaccaagacatctgtcgattgcaccatgtacatctgcggagattccaccgagtgctccaacctactactacagtacggatctttctgtacccagctaaacagagcgttgactggaatcgctgtagagcaggataagaacactcaagaggtattcgcgcaagtcaagcagatctataagactccgccgatcaaggacttcggaggtttcaacttctctcagatcttgccggatccgtccaaaccgtctaagagatctttcatcgaggacctactattcaacaaagtcaccctagctgacgcgggattcatcaaacaatacggagattgcttgggagacattgcggcgagagatctaatttgcgcgcagaagtttaacggattgacagtactaccgccgctactaaccgatgagatgattgcgcagtacacgtctgctctattggcgggaacaattacaagtggatggacatttggagccggtgccgctctacaaattccgtttgctatgcaaatggcgtacagattcaacggaatcggagtaacccagaacgtcttgtacgagaaccagaagctaatcgcgaaccagttcaattccgcgatcggaaagatccaggacagtctatcttctactgcttcggcgttgggaaagctacaggatgtagtaaatcaaaacgcgcaggcgctaaacaccttggtcaagcaactatcctctaacttcggagcgatctcgtccgtcctaaacgacatcttatccagactagataaggtcgaagcggaggtccagatcgatagactaatcactggaagattgcagtccctacagacctacgtaacacagcaactaattagagcggcggagattagagcctctgctaatctagctgcgaccaagatgtccgaatgtgtcttgggacaatccaagagagtggacttctgcggaaagggataccacctaatgtctttcccacaatctgcgccgcatggtgtcgtattcctacatgtaacatatgtgccggcgcaagaaaagaacttcacaacagctccagcgatctgccatgatggaaaagctcatttcccgagagagggagtctttgtctctaacggaactcattggttcgtcacccagagaaacttctacgagccgcagatcatcaccaccgacaacacattcgtctcgggaaactgcgacgtggtcatcggaatcgtaaacaataccgtctacgatccgttgcagccggaactagactccttcaaagaagagttggacaagtacttcaagaaccacacctctccggatgtggacttgggagatatctctggaatcaacgcgtccgtcgtcaacatccagaaagaaatcgatagattgaacgaggtcgcgaagaacttgaacgagtccctaatcgacctacaagagctaggaaaatacgagcagtacatcaagtggccgtggtacatctggctaggattcattgctggactaattgcgatcgtcatggtcaccatcatgctatgctgtatgacctcctgttgctcctgtctaaagggatgttgttcctgcggatcctgttgcaagttcgatgaagatgatagtgaaccggtcctaaagggtgtcaagctacactacacataatatagctaagcttacgcgtttaagaatatacaaataagaaaaacttaggattaaagaataaattaatccttgtccaaaatgagtataactaactctgcaatatacacattcccagaatcatcattctctgaaaatggtcatatagaaccattaccactcaaagtcaatgaacagaggaaagcagtaccccacattagagttgccaagatcggaaatccaccaaaacacggatcccggtatttagatgtcttcttactcggcttcttcgagatggaacgaatcaaagacaaatacgggagtgtgaatgatctcgacagtgacccgagttacaaagtttgtggctctggatcattaccaatcggattggctaagtacactgggaatgaccaggaattgttacaagccgcaaccaaactggatatagaagtgagaagaacagtcaaagcgaaagagatggttgtttacacggtacaaaatataaaaccagaactgtacccgtggtccaatagactaagaaaaggaatgctgttcgatgccaacaaagttgctcttgctcctcaatgtcttccactagataggagcataaaatttagagtaatcttcgtgaattgtacggcaattggatcaataaccttgttcaaaattcctaagtcaatggcatcactatctctacccaacacaatatcaatcaatctgcaggtacacataaaaacaggggttcagactgattctaaagggatagttcaaattttggatgagaaaggcgaaaaatcactgaatttcatggtccatctcggattgatcaaaagaaaagtaggcagaatgtactctgttgaatactgtaaacagaaaatcgagaaaatgagattgatattttctttaggactagttggaggaatcagtcttcatgtcaatgcaactgggtccatatcaaaaacactagcaagtcagctggtattcaaaagagagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctattttagtccggacgtatctattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatatagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtacttggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacaaaagggaaatataaaaaacttaggagcaaagcgtgctcgggaaatggacactgaatctaacaatggcactgtatctgacatactctatcctgagtgtcaccttaactctcctatcgttaaaggtaaaatagcacaattacacactattatgagtctacctcagccttatgatatggatgacgactcaatactagttatcactagacagaaaataaaacttaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcgaaagttcacacaacctataaatcagataaatggtataatccattcaaaacatggtttactatcaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattgtttccaattatgggagaaaagacatttgatgtgatatcgttattagaaccacttgcattatccttaattcaaactcatgatcctgttaaacaactaagaggagcttttttaaatcatgtgttatccgagatggaattaatatttgaatctagagaatcgattaaggaatttctgagtgtagattacattgataaaattttagatatatttaataagtctacaatagatgaaatagcagagattttctctttttttagaacatttgggcatcctccattagaagctagtattgcagcagaaaaggttagaaaatatatgtatattggaaaacaattaaaatttgacactattaataaatgtcatgctatcttctgtacaataataattaacggatatagagagaggcatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaggatttgacaatttatatgaaagataaagcattatctccaaaaaaatcaaattgggacacagtttatcctgcatctaatttactgtaccgtactaacgcatccaacgaatcacgaagattagttgaagtatttatagcagatagtaaatttgatcctcatcagatattggattatgtagaatctggggactggttagatgatccagaatttaatatttcttatagtcttaaagaaaaagagatcaaacaggaaggtagactctttgcaaaaatgacatacaaaatgagagctacacaagttttatcagagaccctacttgcaaataacataggaaaattctttcaagaaaatgggatggtgaagggagagattgaattacttaagagattaacaaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttggattaaataaattgtttaattggttacaccctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcaatacatctagcagctgttagaataggcgtgagggtgactgcaatggttcaaggagacaatcaagctatagctgtaaccacaagagtacccaacaattatgactacagagttaagaaggagatagtttataaagatgtagtgagattttttgattcattaagagaagtgatggatgatctaggtcatgaacttaaattaaatgaaacgattataagtagcaagatgttcatatatagcaaaagaatctattatgatgggagaattcttcctcaagctctaaaagcattatctagatgtgtcttctggtcagagacagtaatagacgaaacaagatcagcatcttcaaatttggcaacatcatttgcaaaagcaattgagaatggttattcacctgttctaggatatgcatgctcaatttttaagaatattcaacaactatatattgcccttgggatgaatatcaatccaactataacacagaatatcagagatcagtattttaggaatccaaattggatgcaatatgcctctttaatacctgctagtgttgggggattcaattacatggccatgtcaagatgttttgtaaggaatattggtgatccatcagttgccgcattggctgatattaaaagatttattaaggcgaatctattagaccgaagtgttctttataggattatgaatcaagaaccaggtgagtcatcttttttggactgggcttcagatccatattcatgcaatttaccacaatctcaaaatataaccaccatgataaaaaatataacagcaaggaatgtattacaagattcaccaaatccattattatctggattattcacaaatacaatgatagaagaagatgaagaattagctgagttcctgatggacaggaaggtaattctccctagagttgcacatgatattctagataattctctcacaggaattagaaatgccatagctggaatgttagatacgacaaaatcactaattcgggttggcataaatagaggaggactgacatatagtttgttgaggaaaatcagtaattacgatctagtacaatatgaaacactaagtaggactttgcgactaattgtaagtgataaaatcaagtatgaagatatgtgttcggtagaccttgccatagcattgcgacaaaagatgtggattcatttatcaggaggaaggatgataagtggacttgaaacgcctgacccattagaattactatctggggtagtaataacaggatcagaacattgtaaaatatgttattcttcagatggcacaaacccatatacttggatgtatttacccggtaatatcaaaataggatcagcagaaacaggtatatcgtcattaagagttccttattttggatcagtcactgatgaaagatctgaagcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttaacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagatactaatcttatttatcaacaaataatgttaacaggattaagtgttttcgaatatttatttagattaaaagaaaccacaggacacaaccctatagttatgcatctgcacatagaagatgagtgttgtattaaagaaagttttaatgatgaacatattaatccagagtctacattagaattaattcgatatcctgaaagtaatgaatttatttatgataaagacccactcaaagatgtggacttatcaaaacttatggttattaaagaccattcttacacaattgatatgaattattgggatgatactgacatcatacatgcaatttcaatatgtactgcaattacaatagcagatactatgtcacaattagatcgagataatttaaaagagataatagttattgcaaatgatgatgatattaatagcttaatcactgaatttttgactcttgacatacttgtatttctcaagacatttggtggattattagtaaatcaatttgcatacactctttatagtctaaaaatagaaggtagggatctcatttgggattatataatgagaacactgagagatacttcccattcaatattaaaagtattatctaatgcattatctcatcctaaagtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatatacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgcggtattagtccacctgaggtaattgatgattgggatccggtagaagatgaaaatatgctggataacattgtcaaaactataaatgataactgtaataaagataataaagggaataaaattaacaatttctggggactagcacttaagaactatcaagtccttaaaatcagatctataacaagtgattctgatgataatgatagactagatgctaatacaagtggtttgacacttcctcaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggtaaaaaattaggaaatgtgacacagattcttaacagggtaaaagtactgttcaatgggaatcctaattcaacatggataggaaatatggaatgtgagagcttaatatggagtgaattaaatgataagtccattggattagtacattgtgatatggaaggagctatcggtaaatcagaagaaactgttctacatgaacattatagtgttataagaattacatacttgattggggatgatgatgttgttttagtttccaaaattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaggaataatgaatggttacatcatgaaatcaaagaaggagaaagagattatggaatcatgagaccatatcatatggcactacaaatctttggatttcaaatcaatttaaatcatctggcgaaagaatttttatcaaccccagatctgactaatatcaacaatataatccaaagttttcagcgaacaataaaggatgttttatttgaatggattaatataactcatgatgataagagacataaattaggcggaagatataacatattcccactgaaaaataagggaaagttaagactgctatcgagaagactagtattaagttggatttcattatcattatcgactcgattacttacaggtcgctttcctgatgaaaaatttgaacatagagcacagactggatatgtatcattagctgatactgatttagaatcattaaagttattgtcgaaaaacatcattaagaattacagagagtgtataggatcaatatcatattggtttctaaccaaagaagttaaaatacttatgaaattgatcggtggtgctaaattattaggaattcccagacaatataaagaacccgaagaccagttattagaaaactacaatcaacatgatgaatttgatatcgattaaaacataaatacaatgaagatatatcctaacctttatctttaagcctaggaatagacaaaaagtaagaaaaacatgtaatatatatataccaaacagagttcttctcttgtttggtgggtcggcatggcatctccacctcctcgcggtccggacctgggcatccgaaggaggacgcacgtccactcggatggctaagggagagcctgcagtagcataaccccttggggcctctaaacgggtcttgaggggttttttgctgaaaggaggaactatatacgcgtcgacgggccccgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcggggcccgtcgacgcgttaatacgactcactatagg

SEQ ID NO: 4 - DNA, 22347 bp, synthetic construct
  misc_feature 1..22347: note = recombinant polynucleotide
  source 1..22347: mol_type = other DNA
accaaacaagagaagaaacttgtctgggaatataaatttaactttaaattaacttaggattaaagacattgactagaaggtcaagaaaagggaactctataatttcaaaaatgttgagcctatttgatacatttaatgcacgtaggcaagaaaacataacaaaatcagccggtggagctatcattcctggacagaaaaatactgtctctatattcgcccttggaccgacaataactgatgataatgagaaaatgacattagctcttctatttctatctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaagatctaaaacggcaaaagtatggaggatttgtggttaagacgagagagatgatatatgaaaagacaactgattggatatttggaagtgacctggattatgatcaggaaactatgttgcagaacggcaggaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagaccagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgccgaagctcaaatgagctcaacactggaagatgaacttggagtgacacacgaatctaaagaaagcttgaagagacatataaggaacataaacagttcagagacatctttccacaaaccgacaggtggatcagccatagagatggcaatagatgaagagccagaacaattcgaacatagagcagatcaagaacaaaatggagaacctcaatcatccataattcaatatgcctgggcagaaggaaatagaagcgatgatcagactgagcaagctacagaatctgacaatatcaagaccgaacaacaaaacatcagagacagactaaacaagagactcaacgacaagaagaaacaaagcagtcaaccacccactaatcccacaaacagaacaaaccaggacgaaatagatgatctgtttaacgcatttggaagcaactaatcgaatcaacattttaatctaaatcaataataaataagaaaaacttaggattaaagaatcctatcataccggaatatagggtggtaaatttagagtctgcttgaaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacttatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctggaggaatccccagaagcatcacagattctaaaaatggaacccaaaacacggaggatattgatctcaatgaaattagaaagatggataaggactctattgaggggaaaatgcgacaatctgcaaatgttccaagcgagatatcaggaagtgatgacatatttacaacagaacaaagtagaaacagtgatcatggaagaagcctggaatctatcagtacacctgatacaagatcaataagtgttgttactgctgcaacaccagatgatgaagaagaaatactaatgaaaaatagtaggacaaagaaaagttcttcaacacatcaagaagatgacaaaagaattaaaaaagggggaaaagggaaagactggtttaagaaatcaaaagataccgacaaccagataccaacatcagactacagatccacatcaaaagggcagaagaaaatctcaaagacaacaaccaccaacaccgacacaaaggggcaaacagaaatacagacagaatcatcagaaacacaatcctcatcatggaatctcatcatcgacaacaacaccgaccggaacgaacagacaagcacaactcctccaacaacaacttccagatcaacttatacaaaagaatcgatccgaacaaactctgaatccaaacccaagacacaaaagacaaatggaaaggaaaggaaggatacagaagagagcaatcgatttacagagagggcaattactctattgcagaatcttggtgtaattcaatccacatcaaaactagatttatatcaagacaaacgagttgtatgtgtagcaaatgtactaaacaatgtagatactgcatcaaagatagatttcctggcaggattagtcataggggtttcaatggacaacgacacaaaattaacacagatacaaaatgaaatgctaaacctcaaagcagatctaaagaaaatggacgaatcacatagaagattgatagaaaatcaaagagaacaactgtcattgatcacgtcactaatttcaaatctcaaaattatgactgagagaggaggaaagaaagaccaaaatgaatccaatgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatattaagttcatacaatgagtcaaatgcaacaagactaatacccaaaaaagtgagcagtacaatgagatcactagttgcagtcatcaacaacagcaatctctcacaaagcacaaaacaatcatacataaacgaactcaaacgttgcaaaaatgatgaagaagtatctgaattaatggacatgttcaatgaagatgtcaacaattgccaatgatccaacaaagaaacgacaccgaacaaacagacaagaaacaacagtagatcaaaacctgtcaacacacacaaaatcaagcagaatgaaacaacagatacttaagaatatacaaataagaaaaacttaggattaaagagccatggtcgtgtttctggtgctgctgcctctggtgagctcccagtgcgtgaacctgaccacaaggacccagctgccccctgcctataccaattccttcacacggggcgtgtactatcccgacaaggtgttccggagcagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaaaatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagcagcagcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagaaatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggaacaggcgtgctgaccgagtccaacaagaagttcctgccttttcagcagttcggcagggacatcgcagataccacagacgccgtgcgcgaccctcagaccctggagatcctggacatcacaccatgctccttcggcggcgtgtctgtgatcacaccaggcaccaatacaagcaaccaggtggccgtgctgtatcaggacgtgaattgtaccgaggtgccagtggcaatccacgcagatcagctgacccctacatggcgggtgtactctaccggcagcaacgtgttccagacaagagccggatgcctgatcggagcagagcacgtgaacaatagctatgagtgcgacatccctatcggcgccggcatctgtgcctcctaccagacccagacaaactccccaaggagagcacggtctgtggccagccagtccatcatcgcctataccatgagcctgggcgccgagaattccgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctgaatagagccctgacaggcatcgccgtggagcaggacaagaacacacaggaggtgttcgcccaggtgaagcaaatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggaaccatcacaagcggatggaccttcggcgcaggagccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctgtctagcaacttcggcgccatctcctctgtgctgaatgacatcctgagccggctggacaaggtggaggcagaggtgcagatcgaccggctgatcacaggcagactgcagtccctgcagacctacgtgacacagcagctgatcagggcagcagagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcacgtgacctacgtgccagcccaggagaagaacttcaccacagcaccagcaatctgccacgatggcaaggcacactttcctagggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggcagctgctgtaagtttgatgaggacgatagcgagcctgtgctgaagggcgtgaagctgcactacacctgatatagctaagcttacgcgtttaagaatatacaaataagaaaaacttaggattaaagaataaattaatccttgtccaaaatgagtataactaactctgcaatatacacattcccagaatcatcattctctgaaaatggtcatatagaaccattaccactcaaagtcaatgaacagaggaaagcagtaccccacattagagttgccaagatcggaaatccaccaaaacacggatcccggtatttagatgtcttcttactcggcttcttcgagatggaacgaatcaaagacaaatacgggagtgtgaatgatctcgacagtgacccgagttacaaagtttgtggctctggatcattaccaatcggattggctaagtacactgggaatgaccaggaattgttacaagccgcaaccaaactggatatagaagtgagaagaacagtcaaagcgaaagagatggttgtttacacggtacaaaatataaaaccagaactgtacccgtggtccaatagactaagaaaaggaatgctgttcgatgccaacaaagttgctcttgctcctcaatgtcttccactagataggagcataaaatttagagtaatcttcgtgaattgtacggcaattggatcaataaccttgttcaaaattcctaagtcaatggcatcactatctctacccaacacaatatcaatcaatctgcaggtacacataaaaacaggggttcagactgattctaaagggatagttcaaattttggatgagaaaggcgaaaaatcactgaatttcatggtccatctcggattgatcaaaagaaaagtaggcagaatgtactctgttgaatactgtaaacagaaaatcgagaaaatgagattgatattttctttaggactagttggaggaatcagtcttcatgtcaatgcaactgggtccatatcaaaaacactagcaagtcagctggtattcaaaagagagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctattttagtccggacgtatctattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatatagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtacttggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacaaaagggaaatataaaaaacttaggagcaaagcgtgctcgggaaatggacactgaatctaacaatggcactgtatctgacatactctatcctgagtgtcaccttaactctcctatcgttaaaggtaaaatagcacaattacacactattatgagtctacctcagccttatgatatggatgacgactcaatactagttatcactagacagaaaataaaacttaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcgaaagttcacacaacctataaatcagataaatggtataatccattcaaaacatggtttactatcaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattgtttccaattatgggagaaaagacatttgatgtgatatcgttattagaaccacttgcattatccttaattcaaactcatgatcctgttaaacaactaagaggagcttttttaaatcatgtgttatccgagatggaattaatatttgaatctagagaatcgattaaggaatttctgagtgtagattacattgataaaattttagatatatttaataagtctacaatagatgaaatagcagagattttctctttttttagaacatttgggcatcctccattagaagctagtattgcagcagaaaaggttagaaaatatatgtatattggaaaacaattaaaatttgacactattaataaatgtcatgctatcttctgtacaataataattaacggatatagagagaggcatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaggatttgacaatttatatgaaagataaagcattatctccaaaaaaatcaaattgggacacagtttatcctgcatctaatttactgtaccgtactaacgcatccaacgaatcacgaagattagttgaagtatttatagcagatagtaaatttgatcctcatcagatattggattatgtagaatctggggactggttagatgatccagaatttaatatttcttatagtcttaaagaaaaagagatcaaacaggaaggtagactctttgcaaaaatgacatacaaaatgagagctacacaagttttatcagagaccctacttgcaaataacataggaaaattctttcaagaaaatgggatggtgaagggagagattgaattacttaagagattaacaaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttggattaaataaattgtttaattggttacaccctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcaatacatctagcagctgttagaataggcgtgagggtgactgcaatggttcaaggagacaatcaagctatagctgtaaccacaagagtacccaacaattatgactacagagttaagaaggagatagtttataaagatgtagtgagattttttgattcattaagagaagtgatggatgatctaggtcatgaacttaaattaaatgaaacgattataagtagcaagatgttcatatatagcaaaagaatctattatgatgggagaattcttcctcaagctctaaaagcattatctagatgtgtcttctggtcagagacagtaatagacgaaacaagatcagcatcttcaaatttggcaacatcatttgcaaaagcaattgagaatggttattcacctgttctaggatatgcatgctcaatttttaagaatattcaacaactatatattgcccttgggatgaatatcaatccaactataacacagaatatcagagatcagtattttaggaatccaaattggatgcaatatgcctctttaatacctgctagtgttgggggattcaattacatggccatgtcaagatgttttgtaaggaatattggtgatccatcagttgccgcattggctgatattaaaagatttattaaggcgaatctattagaccgaagtgttctttataggattatgaatcaagaaccaggtgagtcatcttttttggactgggcttcagatccatattcatgcaatttaccacaatctcaaaatataaccaccatgataaaaaatataacagcaaggaatgtattacaagattcaccaaatccattattatctggattattcacaaatacaatgatagaagaagatgaagaattagctgagttcctgatggacaggaaggtaattctccctagagttgcacatgatattctagataattctctcacaggaattagaaatgccatagctggaatgttagatacgacaaaatcactaattcgggttggcataaatagaggaggactgacatatagtttgttgaggaaaatcagtaattacgatctagtacaatatgaaacactaagtaggactttgcgactaattgtaagtgataaaatcaagtatgaagatatgtgttcggtagaccttgccatagcattgcgacaaaagatgtggattcatttatcaggaggaaggatgataagtggacttgaaacgcctgacccattagaattactatctggggtagtaataacaggatcagaacattgtaaaatatgttattcttcagatggcacaaacccatatacttggatgtatttacccggtaatatcaaaataggatcagcagaaacaggtatatcgtcattaagagttccttattttggatcagtcactgatgaaagatctgaagcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttaacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagatactaatcttatttatcaacaaataatgttaacaggattaagtgttttcgaatatttatttagattaaaagaaaccacaggacacaaccctatagttatgcatctgcacatagaagatgagtgttgtattaaagaaagttttaatgatgaacatattaatccagagtctacattagaattaattcgatatcctgaaagtaatgaatttatttatgataaagacccactcaaagatgtggacttatcaaaacttatggttattaaagaccattcttacacaattgatatgaattattgggatgatactgacatcatacatgcaatttcaatatgtactgcaattacaatagcagatactatgtcacaattagatcgagataatttaaaagagataatagttattgcaaatgatgatgatattaatagcttaatcactgaatttttgactcttgacatacttgtatttctcaagacatttggtggattattagtaaatcaatttgcatacactctttatagtctaaaaatagaaggtagggatctcatttgggattatataatgagaacactgagagatacttcccattcaatattaaaagtattatctaatgcattatctcatcctaaagtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatatacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgcggtattagtccacctgaggtaattgatgattgggatccggtagaagatgaaaatatgctggataacattgtcaaaactataaatgataactgtaataaagataataaagggaataaaattaacaatttctggggactagcacttaagaactatcaagtccttaaaatcagatctataacaagtgattctgatgataatgatagactagatgctaatacaagtggtttgacacttcctcaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggtaaaaaattaggaaatgtgacacagattcttaacagggtaaaagtactgttcaatgggaatcctaattcaacatggataggaaatatggaatgtgagagcttaatatggagtgaattaaatgataagtccattggattagtacattgtgatatggaaggagctatcggtaaatcagaagaaactgttctacatgaacattatagtgttataagaattacatacttgattggggatgatgatgttgttttagtttccaaaattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaggaataatgaatggttacatcatgaaatcaaagaaggagaaagagattatggaatcatgagaccatatcatatggcactacaaatctttggatttcaaatcaatttaaatcatctggcgaaagaatttttatcaaccccagatctgactaatatcaacaatataatccaaagttttcagcgaacaataaaggatgttttatttgaatggattaatataactcatgatgataagagacataaattaggcggaagatataacatattcccactgaaaaataagggaaagttaagactgctatcgagaagactagtattaagttggatttcattatcattatcgactcgattacttacaggtcgctttcctgatgaaaaatttgaacatagagcacagactggatatgtatcattagctgatactgatttagaatcattaaagttattgtcgaaaaacatcattaagaattacagagagtgtataggatcaatatcatattggtttctaaccaaagaagttaaaatacttatgaaattgatcggtggtgctaaattattaggaattcccagacaatataaagaacccgaagaccagttattagaaaactacaatcaacatgatgaatttgatatcgattaaaacataaatacaatgaagatatatcctaacctttatctttaagcctaggaatagacaaaaagtaagaaaaacatgtaatatatatataccaaacagagttcttctcttgtttggtgggtcggcatggcatctccacctcctcgcggtccggacctgggcatccgaaggaggacgcacgtccactcggatggctaagggagagcctgcagtagcataaccccttggggcctctaaacgggtcttgaggggttttttgctgaaaggaggaactatatacgcgtcgacgggccccgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcggggcccgtcgacgcgttaatacgactcactatagg

SEQ ID NO: 5 - DNA, 19353 bp, synthetic construct
  misc_feature 1..19353: note = recombinant polynucleotide
  source 1..19353: mol_type = other DNA
accaaacaagagaagaaacttgtctgggaatataaatttaactttaaattaacttaggattaaagacattgactagaaggtcaagaaaagggaactctataatttcaaaaatgttgagcctatttgatacatttaatgcacgtaggcaagaaaacataacaaaatcagccggtggagctatcattcctggacagaaaaatactgtctctatattcgcccttggaccgacaataactgatgataatgagaaaatgacattagctcttctatttctatctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaagatctaaaacggcaaaagtatggaggatttgtggttaagacgagagagatgatatatgaaaagacaactgattggatatttggaagtgacctggattatgatcaggaaactatgttgcagaacggcaggaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagaccagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgccgaagctcaaatgagctcaacactggaagatgaacttggagtgacacacgaatctaaagaaagcttgaagagacatataaggaacataaacagttcagagacatctttccacaaaccgacaggtggatcagccatagagatggcaatagatgaagagccagaacaattcgaacatagagcagatcaagaacaaaatggagaacctcaatcatccataattcaatatgcctgggcagaaggaaatagaagcgatgatcagactgagcaagctacagaatctgacaatatcaagaccgaacaacaaaacatcagagacagactaaacaagagactcaacgacaagaagaaacaaagcagtcaaccacccactaatcccacaaacagaacaaaccaggacgaaatagatgatctgtttaacgcatttggaagcaactaatcgaatcaacattttaatctaaatcaataataaataagaaaaacttaggattaaagaatcctatcataccggaatatagggtggtaaatttagagtctgcttgaaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacttatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctggaggaatccccagaagcatcacagattctaaaaatggaacccaaaacacggaggatattgatctcaatgaaattagaaagatggataaggactctattgaggggaaaatgcgacaatctgcaaatgttccaagcgagatatcaggaagtgatgacatatttacaacagaacaaagtagaaacagtgatcatggaagaagcctggaatctatcagtacacctgatacaagatcaataagtgttgttactgctgcaacaccagatgatgaagaagaaatactaatgaaaaatagtaggacaaagaaaagttcttcaacacatcaagaagatgacaaaagaattaaaaaagggggaaaagggaaagactggtttaagaaatcaaaagataccgacaaccagataccaacatcagactacagatccacatcaaaagggcagaagaaaatctcaaagacaacaaccaccaacaccgacacaaaggggcaaacagaaatacagacagaatcatcagaaacacaatcctcatcatggaatctcatcatcgacaacaacaccgaccggaacgaacagacaagcacaactcctccaacaacaacttccagatcaacttatacaaaagaatcgatccgaacaaactctgaatccaaacccaagacacaaaagacaaatggaaaggaaaggaaggatacagaagagagcaatcgatttacagagagggcaattactctattgcagaatcttggtgtaattcaatccacatcaaaactagatttatatcaagacaaacgagttgtatgtgtagcaaatgtactaaacaatgtagatactgcatcaaagatagatttcctggcaggattagtcataggggtttcaatggacaacgacacaaaattaacacagatacaaaatgaaatgctaaacctcaaagcagatctaaagaaaatggacgaatcacatagaagattgatagaaaatcaaagagaacaactgtcattgatcacgtcactaatttcaaatctcaaaattatgactgagagaggaggaaagaaagaccaaaatgaatccaatgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatattaagttcatacaatgagtcaaatgcaacaagactaatacccaaaaaagtgagcagtacaatgagatcactagttgcagtcatcaacaacagcaatctctcacaaagcacaaaacaatcatacataaacgaactcaaacgttgcaaaaatgatgaagaagtatctgaattaatggacatgttcaatgaagatgtcaacaattgccaatgatccaacaaagaaacgacaccgaacaaacagacaagaaacaacagtagatcaaaacctgtcaacacacacaaaatcaagcagaatgaaacaacagatacttaagaatatacaaataagaaaaacttaggattaaagagccatggttagagtacagccgaccgaatccatcgtcagatttccgaacatcacgaacctatgtccgttcggagaagtgttcaacgcgacaagatttgcgtctgtctatgcgtggaacagaaaaagaatcagtaactgcgtcgcggactactccgtcctatacaactctgcctctttctccacgttcaaatgctacggtgtatctccgacaaagctaaacgatctatgcttcaccaacgtctacgcggactccttcgtaatcagaggagatgaagttagacagattgcgccgggacaaactggaaagatcgcggattataactacaagctaccggacgacttcaccggatgtgtaattgcgtggaattcgaacaacctagactccaaagtcggaggaaactacaactacttgtacagactattcagaaagtccaacctaaagccgttcgagagagacatctccaccgaaatctatcaggctggatctacaccgtgtaatggtgtcgaaggattcaactgctacttcccgctacagtcttacggatttcaaccgacaaacggtgtaggatatcagccgtacagagtcgtcgtactatccttcgaactactacatgctccggcgacagtatgtggaccgaaaaagtctaccaacctagtcaagaacaaatgcgtcaactttaacttcaacggactaaccggaaccggtgtcctaaccgaatctaacaagaagtttctaccgttccagcagttcggaagagatatcgcggatacaacagacgctgtcagagatccgcaaaccttggagatcctagatatcacaccgtgttcttaatatagctaagcttacgcgtttaagaatatacaaataagaaaaacttaggattaaagaataaattaatccttgtccaaaatgagtataactaactctgcaatatacacattcccagaatcatcattctctgaaaatggtcatatagaaccattaccactcaaagtcaatgaacagaggaaagcagtaccccacattagagttgccaagatcggaaatccaccaaaacacggatcccggtatttagatgtcttcttactcggcttcttcgagatggaacgaatcaaagacaaatacgggagtgtgaatgatctcgacagtgacccgagttacaaagtttgtggctctggatcattaccaatcggattggctaagtacactgggaatgaccaggaattgttacaagccgcaaccaaactggatatagaagtgagaagaacagtcaaagcgaaagagatggttgtttacacggtacaaaatataaaaccagaactgtacccgtggtccaatagactaagaaaaggaatgctgttcgatgccaacaaagttgctcttgctcctcaatgtcttccactagataggagcataaaatttagagtaatcttcgtgaattgtacggcaattggatcaataaccttgttcaaaattcctaagtcaatggcatcactatctctacccaacacaatatcaatcaatctgcaggtacacataaaaacaggggttcagactgattctaaagggatagttcaaattttggatgagaaaggcgaaaaatcactgaatttcatggtccatctcggattgatcaaaagaaaagtaggcagaatgtactctgttgaatactgtaaacagaaaatcgagaaaatgagattgatattttctttaggactagttggaggaatcagtcttcatgtcaatgcaactgggtccatatcaaaaacactagcaagtcagctggtattcaaaagagagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctattttagtccggacgtatctattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatatagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtacttggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacaaaagggaaatataaaaaacttaggagcaaagcgtgctcgggaaatggacactgaatctaacaatggcactgtatctgacatactctatcctgagtgtcaccttaactctcctatcgttaaaggtaaaatagcacaattacacactattatgagtctacctcagccttatgatatggatgacgactcaatactagttatcactagacagaaaataaaacttaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcgaaagttcacacaacctataaatcagataaatggtataatccattcaaaacatggtttactatcaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattgtttccaattatgggagaaaagacatttgatgtgatatcgttattagaaccacttgcattatccttaattcaaactcatgatcctgttaaacaactaagaggagcttttttaaatcatgtgttatccgagatggaattaatatttgaatctagagaatcgattaaggaatttctgagtgtagattacattgataaaattttagatatatttaataagtctacaatagatgaaatagcagagattttctctttttttagaacatttgggcatcctccattagaagctagtattgcagcagaaaaggttagaaaatatatgtatattggaaaacaattaaaatttgacactattaataaatgtcatgctatcttctgtacaataataattaacggatatagagagaggcatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaggatttgacaatttatatgaaagataaagcattatctccaaaaaaatcaaattgggacacagtttatcctgcatctaatttactgtaccgtactaacgcatccaacgaatcacgaagattagttgaagtatttatagcagatagtaaatttgatcctcatcagatattggattatgtagaatctggggactggttagatgatccagaatttaatatttcttatagtcttaaagaaaaagagatcaaacaggaaggtagactctttgcaaaaatgacatacaaaatgagagctacacaagttttatcagagaccctacttgcaaataacataggaaaattctttcaagaaaatgggatggtgaagggagagattgaattacttaagagattaacaaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttggattaaataaattgtttaattggttacaccctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcaatacatctagcagctgttagaataggcgtgagggtgactgcaatggttcaaggagacaatcaagctatagctgtaaccacaagagtacccaacaattatgactacagagttaagaaggagatagtttataaagatgtagtgagattttttgattcattaagagaagtgatggatgatctaggtcatgaacttaaattaaatgaaacgattataagtagcaagatgttcatatatagcaaaagaatctattatgatgggagaattcttcctcaagctctaaaagcattatctagatgtgtcttctggtcagagacagtaatagacgaaacaagatcagcatcttcaaatttggcaacatcatttgcaaaagcaattgagaatggttattcacctgttctaggatatgcatgctcaatttttaagaatattcaacaactatatattgcccttgggatgaatatcaatccaactataacacagaatatcagagatcagtattttaggaatccaaattggatgcaatatgcctctttaatacctgctagtgttgggggattcaattacatggccatgtcaagatgttttgtaaggaatattggtgatccatcagttgccgcattggctgatattaaaagatttattaaggcgaatctattagaccgaagtgttctttataggattatgaatcaagaaccaggtgagtcatcttttttggactgggcttcagatccatattcatgcaatttaccacaatctcaaaatataaccaccatgataaaaaatataacagcaaggaatgtattacaagattcaccaaatccattattatctggattattcacaaatacaatgatagaagaagatgaagaattagctgagttcctgatggacaggaaggtaattctccctagagttgcacatgatattctagataattctctcacaggaattagaaatgccatagctggaatgttagatacgacaaaatcactaattcgggttggcataaatagaggaggactgacatatagtttgttgaggaaaatcagtaattacgatctagtacaatatgaaacactaagtaggactttgcgactaattgtaagtgataaaatcaagtatgaagatatgtgttcggtagaccttgccatagcattgcgacaaaagatgtggattcatttatcaggaggaaggatgataagtggacttgaaacgcctgacccattagaattactatctggggtagtaataacaggatcagaacattgtaaaatatgttattcttcagatggcacaaacccatatacttggatgtatttacccggtaatatcaaaataggatcagcagaaacaggtatatcgtcattaagagttccttattttggatcagtcactgatgaaagatctgaagcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttaacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagatactaatcttatttatcaacaaataatgttaacaggattaagtgttttcgaatatttatttagattaaaagaaaccacaggacacaaccctatagttatgcatctgcacatagaagatgagtgttgtattaaagaaagttttaatgatgaacatattaatccagagtctacattagaattaattcgatatcctgaaagtaatgaatttatttatgataaagacccactcaaagatgtggacttatcaaaacttatggttattaaagaccattcttacacaattgatatgaattattgggatgatactgacatcatacatgcaatttcaatatgtactgcaattacaatagcagatactatgtcacaattagatcgagataatttaaaagagataatagttattgcaaatgatgatgatattaatagcttaatcactgaatttttgactcttgacatacttgtatttctcaagacatttggtggattattagtaaatcaatttgcatacactctttatagtctaaaaatagaaggtagggatctcatttgggattatataatgagaacactgagagatacttcccattcaatattaaaagtattatctaatgcattatctcatcctaaagtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatatacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgcggtattagtccacctgaggtaattgatgattgggatccggtagaagatgaaaatatgctggataacattgtcaaaactataaatgataactgtaataaagataataaagggaataaaattaacaatttctggggactagcacttaagaactatcaagtccttaaaatcagatctataacaagtgattctgatgataatgatagactagatgctaatacaagtggtttgacacttcctcaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggtaaaaaattaggaaatgtgacacagattcttaacagggtaaaagtactgttcaatgggaatcctaattcaacatggataggaaatatggaatgtgagagcttaatatggagtgaattaaatgataagtccattggattagtacattgtgatatggaaggagctatcggtaaatcagaagaaactgttctacatgaacattatagtgttataagaattacatacttgattggggatgatgatgttgttttagtttccaaaattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaggaataatgaatggttacatcatgaaatcaaagaaggagaaagagattatggaatcatgagaccatatcatatggcactacaaatctttggatttcaaatcaatttaaatcatctggcgaaagaatttttatcaaccccagatctgactaatatcaacaatataatccaaagttttcagcgaacaataaaggatgttttatttgaatggattaatataactcatgatgataagagacataaattaggcggaagatataacatattcccactgaaaaataagggaaagttaagactgctatcgagaagactagtattaagttggatttcattatcattatcgactcgattacttacaggtcgctttcctgatgaaaaatttgaacatagagcacagactggatatgtatcattagctgatactgatttagaatcattaaagttattgtcgaaaaacatcattaagaattacagagagtgtataggatcaatatcatattggtttctaaccaaagaagttaaaatacttatgaaattgatcggtggtgctaaattattaggaattcccagacaatataaagaacccgaagaccagttattagaaaactacaatcaacatgatgaatttgatatcgattaaaacataaatacaatgaagatatatcctaacctttatctttaagcctaggaatagacaaaaagtaagaaaaacatgtaatatatatataccaaacagagttcttctcttgtttggtgggtcggcatggcatctccacctcctcgcggtccggacctgggcatccgaaggaggacgcacgtccactcggatggctaagggagagcctgcagtagcataaccccttggggcctctaaacgggtcttgaggggttttttgctgaaaggaggaactatatacgcgtcgacgggccccgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcggggcccgtcgacgcgttaatacgactcactatagg

SEQ ID NO: 6 - DNA, 19353 bp, synthetic construct
  misc_feature 1..19353: note = recombinant polynucleotide
  source 1..19353: mol_type = other DNA
accaaacaagagaagaaacttgtctgggaatataaatttaactttaaattaacttaggattaaagacattgactagaaggtcaagaaaagggaactctataatttcaaaaatgttgagcctatttgatacatttaatgcacgtaggcaagaaaacataacaaaatcagccggtggagctatcattcctggacagaaaaatactgtctctatattcgcccttggaccgacaataactgatgataatgagaaaatgacattagctcttctatttctatctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaagatctaaaacggcaaaagtatggaggatttgtggttaagacgagagagatgatatatgaaaagacaactgattggatatttggaagtgacctggattatgatcaggaaactatgttgcagaacggcaggaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagaccagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgccgaagctcaaatgagctcaacactggaagatgaacttggagtgacacacgaatctaaagaaagcttgaagagacatataaggaacataaacagttcagagacatctttccacaaaccgacaggtggatcagccatagagatggcaatagatgaagagccagaacaattcgaacatagagcagatcaagaacaaaatggagaacctcaatcatccataattcaatatgcctgggcagaaggaaatagaagcgatgatcagactgagcaagctacagaatctgacaatatcaagaccgaacaacaaaacatcagagacagactaaacaagagactcaacgacaagaagaaacaaagcagtcaaccacccactaatcccacaaacagaacaaaccaggacgaaatagatgatctgtttaacgcatttggaagcaactaatcgaatcaacattttaatctaaatcaataataaataagaaaaacttaggattaaagaatcctatcataccggaatatagggtggtaaatttagagtctgcttgaaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacttatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctggaggaatccccagaagcatcacagattctaaaaatggaacccaaaacacggaggatattgatctcaatgaaattagaaagatggataaggactctattgaggggaaaatgcgacaatctgcaaatgttccaagcgagatatcaggaagtgatgacatatttacaacagaacaaagtagaaacagtgatcatggaagaagcctggaatctatcagtacacctgatacaagatcaataagtgttgttactgctgcaacaccagatgatgaagaagaaatactaatgaaaaatagtaggacaaagaaaagttcttcaacacatcaagaagatgacaaaagaattaaaaaagggggaaaagggaaagactggtttaagaaatcaaaagataccgacaaccagataccaacatcagactacagatccacatcaaaagggcagaagaaaatctcaaagacaacaaccaccaacaccgacacaaaggggcaaacagaaatacagacagaatcatcagaaacacaatcctcatcatggaatctcatcatcgacaacaacaccgaccggaacgaacagacaagcacaactcctccaacaacaacttccagatcaacttatacaaaagaatcgatccgaacaaactctgaatccaaacccaagacacaaaagacaaatggaaaggaaaggaaggatacagaagagagcaatcgatttacagagagggcaattactctattgcagaatcttggtgtaattcaatccacatcaaaactagatttatatcaagacaaacgagttgtatgtgtagcaaatgtactaaacaatgtagatactgcatcaaagatagatttcctggcaggattagtcataggggtttcaatggacaacgacacaaaattaacacagatacaaaatgaaatgctaaacctcaaagcagatctaaagaaaatggacgaatcacatagaagattgatagaaaatcaaagagaacaactgtcattgatcacgtcactaatttcaaatctcaaaattatgactgagagaggaggaaagaaagaccaaaatgaatccaatgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatattaagttcatacaatgagtcaaatgcaacaagactaatacccaaaaaagtgagcagtacaatgagatcactagttgcagtcatcaacaacagcaatctctcacaaagcacaaaacaatcatacataaacgaactcaaacgttgcaaaaatgatgaagaagtatctgaattaatggacatgttcaatgaagatgtcaacaattgccaatgatccaacaaagaaacgacaccgaacaaacagacaagaaacaacagtagatcaaaacctgtcaacacacacaaaatcaagcagaatgaaacaacagatacttaagaatatacaaataagaaaaacttaggattaaagagccatggtcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagaaatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggaacaggcgtgctgaccgagtccaacaagaagttcctgccttttcagcagttcggcagggacatcgcagataccacagacgccgtgcgcgaccctcagaccctggagatcctggacatcacaccatgctcctaatatagctaagcttacgcgtttaagaatatacaaataagaaaaacttaggattaaagaataaattaatccttgtccaaaatgagtataactaactctgcaatatacacattcccagaatcatcattctctgaaaatggtcatatagaaccattaccactcaaagtcaatgaacagaggaaagcagtaccccacattagagttgccaagatcggaaatccaccaaaacacggatcccggtatttagatgtcttcttactcggcttcttcgagatggaacgaatcaaagacaaatacgggagtgtgaatgatctcgacagtgacccgagttacaaagtttgtggctctggatcattaccaatcggattggctaagtacactgggaatgaccaggaattgttacaagccgcaaccaaactggatatagaagtgagaagaacagtcaaagcgaaagagatggttgtttacacggtacaaaatataaaaccagaactgtacccgtggtccaatagactaagaaaaggaatgctgttcgatgccaacaaagttgctcttgctcctcaatgtcttccactagataggagcataaaatttagagtaatcttcgtgaattgtacggcaattggatcaataaccttgttcaaaattcctaagtcaatggcatcactatctctacccaacacaatatcaatcaatctgcaggtacacataaaaacaggggttcagactgattctaaagggatagttcaaattttggatgagaaaggcgaaaaatcactgaatttcatggtccatctcggattgatcaaaagaaaagtaggcagaatgtactctgttgaatactgtaaacagaaaatcgagaaaatgagattgatattttctttaggactagttggaggaatcagtcttcatgtcaatgcaactgggtccatatcaaaaacactagcaagtcagctggtattcaaaagagagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctattttagtccggacgtatctattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatatagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtacttggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacaaaagggaaatataaaaaacttaggagcaaagcgtgctcgggaaatggacactgaatctaacaatggcactgtatctgacatactctatcctgagtgtcaccttaactctcctatcgttaaaggtaaaatagcacaattacacactattatgagtctacctcagccttatgatatggatgacgactcaatactagttatcactagacagaaaataaaacttaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcgaaagttcacacaacctataaatcagataaatggtataatccattcaaaacatggtttactatcaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattgtttccaattatgggagaaaagacatttgatgtgatatcgttattagaaccacttgcattatccttaattcaaactcatgatcctgttaaacaactaagaggagcttttttaaatcatgtgttatccgagatggaattaatatttgaatctagagaatcgattaaggaatttctgagtgtagattacattgataaaattttagatatatttaataagtctacaatagatgaaatagcagagattttctctttttttagaacatttgggcatcctccattagaagctagtattgcagcagaaaaggttagaaaatatatgtatattggaaaacaattaaaatttgacactattaataaatgtcatgctatcttctgtacaataataattaacggatatagagagaggcatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaggatttgacaatttatatgaaagataaagcattatctccaaaaaaatcaaattgggacacagtttatcctgcatctaatttactgtaccgtactaacgcatccaacgaatcacgaagattagttgaagtatttatagcagatagtaaatttgatcctcatcagatattggattatgtagaatctggggactggttagatgatccagaatttaatatttcttatagtcttaaagaaaaagagatcaaacaggaaggtagactctttgcaaaaatgacatacaaaatgagagctacacaagttttatcagagaccctacttgcaaataacataggaaaattctttcaagaaaatgggatggtgaagggagagattgaattacttaagagattaacaaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttggattaaataaattgtttaattggttacaccctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcaatacatctagcagctgttagaataggcgtgagggtgactgcaatggttcaaggagacaatcaagctatagctgtaaccacaagagtacccaacaattatgactacagagttaagaaggagatagtttataaagatgtagtgagattttttgattcattaagagaagtgatggatgatctaggtcatgaacttaaattaaatgaaacgattataagtagcaagatgttcatatatagcaaaagaatctattatgatgggagaattcttcctcaagctctaaaagcattatctagatgtgtcttctggtcagagacagtaatagacgaaacaagatcagcatcttcaaatttggcaacatcatttgcaaaagcaattgagaatggttattcacctgttctaggatatgcatgctcaatttttaagaatattcaacaactatatattgcccttgggatgaatatcaatccaactataacacagaatatcagagatcagtattttaggaatccaaattggatgcaatatgcctctttaatacctgctagtgttgggggattcaattacatggccatgtcaagatgttttgtaaggaatattggtgatccatcagttgccgcattggctgatattaaaagatttattaaggcgaatctattagaccgaagtgttctttataggattatgaatcaagaaccaggtgagtcatcttttttggactgggcttcagatccatattcatgcaatttaccacaatctcaaaatataaccaccatgataaaaaatataacagcaaggaatgtattacaagattcaccaaatccattattatctggattattcacaaatacaatgatagaagaagatgaagaattagctgagttcctgatggacaggaaggtaattctccctagagttgcacatgatattctagataattctctcacaggaattagaaatgccatagctggaatgttagatacgacaaaatcactaattcgggttggcataaatagaggaggactgacatatagtttgttgaggaaaatcagtaattacgatctagtacaatatgaaacactaagtaggactttgcgactaattgtaagtgataaaatcaagtatgaagatatgtgttcggtagaccttgccatagcattgcgacaaaagatgtggattcatttatcaggaggaaggatgataagtggacttgaaacgcctgacccattagaattactatctggggtagtaataacaggatcagaacattgtaaaatatgttattcttcagatggcacaaacccatatacttggatgtatttacccggtaatatcaaaataggatcagcagaaacaggtatatcgtcattaagagttccttattttggatcagtcactgatgaaagatctgaagcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttaacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagatactaatcttatttatcaacaaataatgttaacaggattaagtgttttcgaatatttatttagattaaaagaaaccacaggacacaaccctatagttatgcatctgcacatagaagatgagtgttgtattaaagaaagttttaatgatgaacatattaatccagagtctacattagaattaattcgatatcctgaaagtaatgaatttatttatgataaagacccactcaaagatgtggacttatcaaaacttatggttattaaagaccattcttacacaattgatatgaattattgggatgatactgacatcatacatgcaatttcaatatgtactgcaattacaatagcagatactatgtcacaattagatcgagataatttaaaagagataatagttattgcaaatgatgatgatattaatagcttaatcactgaatttttgactcttgacatacttgtatttctcaagacatttggtggattattagtaaatcaatttgcatacactctttatagtctaaaaatagaaggtagggatctcatttgggattatataatgagaacactgagagatacttcccattcaatattaaaagtattatctaatgcattatctcatcctaaagtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatatacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgcggtattagtccacctgaggtaattgatgattgggatccggtagaagatgaaaatatgctggataacattgtcaaaactataaatgataactgtaataaagataataaagggaataaaattaacaatttctggggactagcacttaagaactatcaagtccttaaaatcagatctataacaagtgattctgatgataatgatagactagatgctaatacaagtggtttgacacttcctcaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggtaaaaaattaggaaatgtgacacagattcttaacagggtaaaagtactgttcaatgggaatcctaattcaacatggataggaaatatggaatgtgagagcttaatatggagtgaattaaatgataagtccattggattagtacattgtgatatggaaggagctatcggtaaatcagaagaaactgttctacatgaacattatagtgttataagaattacatacttgattggggatgatgatgttgttttagtttccaaaattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaggaataatgaatggttacatcatgaaatcaaagaaggagaaagagattatggaatcatgagaccatatcatatggcactacaaatctttggatttcaaatcaatttaaatcatctggcgaaagaatttttatcaaccccagatctgactaatatcaacaatataatccaaagttttcagcgaacaataaaggatgttttatttgaatggattaatataactcatgatgataagagacataaattaggcggaagatataacatattcccactgaaaaataagggaaagttaagactgctatcgagaagactagtattaagttggatttcattatcattatcgactcgattacttacaggtcgctttcctgatgaaaaatttgaacatagagcacagactggatatgtatcattagctgatactgatttagaatcattaaagttattgtcgaaaaacatcattaagaattacagagagtgtataggatcaatatcatattggtttctaaccaaagaagttaaaatacttatgaaattgatcggtggtgctaaattattaggaattcccagacaatataaagaacccgaagaccagttattagaaaactacaatcaacatgatgaatttgatatcgattaaaacataaatacaatgaagatatatcctaacctttatctttaagcctaggaatagacaaaaagtaagaaaaacatgtaatatatatataccaaacagagttcttctcttgtttggtgggtcggcatggcatctccacctcctcgcggtccggacctgggcatccgaaggaggacgcacgtccactcggatggctaagggagagcctgcagtagcataaccccttggggcctctaaacgggtcttgaggggttttttgctgaaaggaggaactatatacgcgtcgacgggccccgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcggggcccgtcgacgcgttaatacgactcactatagg

SEQ ID NO: 7 - DNA, 19167 bp, synthetic construct
  misc_feature 1..19167: note = recombinant polynucleotide
  source 1..19167: mol_type = other DNA
accaaacaagagaagaaacttgtctgggaatataaatttaactttaaattaacttaggattaaagacattgactagaaggtcaagaaaagggaactctataatttcaaaaatgttgagcctatttgatacatttaatgcacgtaggcaagaaaacataacaaaatcagccggtggagctatcattcctggacagaaaaatactgtctctatattcgcccttggaccgacaataactgatgataatgagaaaatgacattagctcttctatttctatctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaagatctaaaacggcaaaagtatggaggatttgtggttaagacgagagagatgatatatgaaaagacaactgattggatatttggaagtgacctggattatgatcaggaaactatgttgcagaacggcaggaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagaccagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgccgaagctcaaatgagctcaacactggaagatgaacttggagtgacacacgaatctaaagaaagcttgaagagacatataaggaacataaacagttcagagacatctttccacaaaccgacaggtggatcagccatagagatggcaatagatgaagagccagaacaattcgaacatagagcagatcaagaacaaaatggagaacctcaatcatccataattcaatatgcctgggcagaaggaaatagaagcgatgatcagactgagcaagctacagaatctgacaatatcaagaccgaacaacaaaacatcagagacagactaaacaagagactcaacgacaagaagaaacaaagcagtcaaccacccactaatcccacaaacagaacaaaccaggacgaaatagatgatctgtttaacgcatttggaagcaactaatcgaatcaacattttaatctaaatcaataataaataagaaaaacttaggattaaagaatcctatcataccggaatatagggtggtaaatttagagtctgcttgaaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacttatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctggaggaatccccagaagcatcacagattctaaaaatggaacccaaaacacggaggatattgatctcaatgaaattagaaagatggataaggactctattgaggggaaaatgcgacaatctgcaaatgttccaagcgagatatcaggaagtgatgacatatttacaacagaacaaagtagaaacagtgatcatggaagaagcctggaatctatcagtacacctgatacaagatcaataagtgttgttactgctgcaacaccagatgatgaagaagaaatactaatgaaaaatagtaggacaaagaaaagttcttcaacacatcaagaagatgacaaaagaattaaaaaagggggaaaagggaaagactggtttaagaaatcaaaagataccgacaaccagataccaacatcagactacagatccacatcaaaagggcagaagaaaatctcaaagacaacaaccaccaacaccgacacaaaggggcaaacagaaatacagacagaatcatcagaaacacaatcctcatcatggaatctcatcatcgacaacaacaccgaccggaacgaacagacaagcacaactcctccaacaacaacttccagatcaacttatacaaaagaatcgatccgaacaaactctgaatccaaacccaagacacaaaagacaaatggaaaggaaaggaaggatacagaagagagcaatcgatttacagagagggcaattactctattgcagaatcttggtgtaattcaatccacatcaaaactagatttatatcaagacaaacgagttgtatgtgtagcaaatgtactaaacaatgtagatactgcatcaaagatagatttcctggcaggattagtcataggggtttcaatggacaacgacacaaaattaacacagatacaaaatgaaatgctaaacctcaaagcagatctaaagaaaatggacgaatcacatagaagattgatagaaaatcaaagagaacaactgtcattgatcacgtcactaatttcaaatctcaaaattatgactgagagaggaggaaagaaagaccaaaatgaatccaatgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatattaagttcatacaatgagtcaaatgcaacaagactaatacccaaaaaagtgagcagtacaatgagatcactagttgcagtcatcaacaacagcaatctctcacaaagcacaaaacaatcatacataaacgaactcaaacgttgcaaaaatgatgaagaagtatctgaattaatggacatgttcaatgaagatgtcaacaattgccaatgatccaacaaagaaacgacaccgaacaaacagacaagaaacaacagtagatcaaaacctgtcaacacacacaaaatcaagcagaatgaaacaacagatacttaagaatatacaaataagaaaaacttaggattaaagagccatggttagagtacagccgaccgaatccatcgtcagatttccgaacatcacgaacctatgtccgttcggagaagtgttcaacgcgacaagatttgcgtctgtctatgcgtggaacagaaaaagaatcagtaactgcgtcgcggactactccgtcctatacaactctgcctctttctccacgttcaaatgctacggtgtatctccgacaaagctaaacgatctatgcttcaccaacgtctacgcggactccttcgtaatcagaggagatgaagttagacagattgcgccgggacaaactggaaagatcgcggattataactacaagctaccggacgacttcaccggatgtgtaattgcgtggaattcgaacaacctagactccaaagtcggaggaaactacaactacttgtacagactattcagaaagtccaacctaaagccgttcgagagagacatctccaccgaaatctatcaggctggatctacaccgtgtaatggtgtcgaaggattcaactgctacttcccgctacagtcttacggatttcaaccgacaaacggtgtaggatatcagccgtacagagtcgtcgtactatccttcgaactactacatgctccggcgacagtatgtggaccgaaaaagtaatatagctaagcttacgcgtttaagaatatacaaataagaaaaacttaggattaaagaataaattaatccttgtccaaaatgagtataactaactctgcaatatacacattcccagaatcatcattctctgaaaatggtcatatagaaccattaccactcaaagtcaatgaacagaggaaagcagtaccccacattagagttgccaagatcggaaatccaccaaaacacggatcccggtatttagatgtcttcttactcggcttcttcgagatggaacgaatcaaagacaaatacgggagtgtgaatgatctcgacagtgacccgagttacaaagtttgtggctctggatcattaccaatcggattggctaagtacactgggaatgaccaggaattgttacaagccgcaaccaaactggatatagaagtgagaagaacagtcaaagcgaaagagatggttgtttacacggtacaaaatataaaaccagaactgtacccgtggtccaatagactaagaaaaggaatgctgttcgatgccaacaaagttgctcttgctcctcaatgtcttccactagataggagcataaaatttagagtaatcttcgtgaattgtacggcaattggatcaataaccttgttcaaaattcctaagtcaatggcatcactatctctacccaacacaatatcaatcaatctgcaggtacacataaaaacaggggttcagactgattctaaagggatagttcaaattttggatgagaaaggcgaaaaatcactgaatttcatggtccatctcggattgatcaaaagaaaagtaggcagaatgtactctgttgaatactgtaaacagaaaatcgagaaaatgagattgatattttctttaggactagttggaggaatcagtcttcatgtcaatgcaactgggtccatatcaaaaacactagcaagtcagctggtattcaaaagagagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctattttagtccggacgtatctattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatatagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtacttggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacaaaagggaaatataaaaaacttaggagcaaagcgtgctcgggaaatggacactgaatctaacaatggcactgtatctgacatactctatcctgagtgtcaccttaactctcctatcgttaaaggtaaaatagcacaattacacactattatgagtctacctcagccttatgatatggatgacgactcaatactagttatcactagacagaaaataaaacttaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcgaaagttcacacaacctataaatcagataaatggtataatccattcaaaacatggtttactatcaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattgtttccaattatgggagaaaagacatttgatgtgatatcgttattagaaccacttgcattatccttaattcaaactcatgatcctgttaaacaactaagaggagcttttttaaatcatgtgttatccgagatggaattaatatttgaatctagagaatcgattaaggaatttctgagtgtagattacattgataaaattttagatatatttaataagtctacaatagatgaaatagcagagattttctctttttttagaacatttgggcatcctccattagaagctagtattgcagcagaaaaggttagaaaatatatgtatattggaaaacaattaaaatttgacactattaataaatgtcatgctatcttctgtacaataataattaacggatatagagagaggcatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaggatttgacaatttatatgaaagataaagcattatctccaaaaaaatcaaattgggacacagtttatcctgcatctaatttactgtaccgtactaacgcatccaacgaatcacgaagattagttgaagtatttatagcagatagtaaatttgatcctcatcagatattggattatgtagaatctggggactggttagatgatccagaatttaatatttcttatagtcttaaagaaaaagagatcaaacaggaaggtagactctttgcaaaaatgacatacaaaatgagagctacacaagttttatcagagaccctacttgcaaataacataggaaaattctttcaagaaaatgggatggtgaagggagagattgaattacttaagagattaacaaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttggattaaataaattgtttaattggttacaccctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcaatacatctagcagctgttagaataggcgtgagggtgactgcaatggttcaaggagacaatcaagctatagctgtaaccacaagagtacccaacaattatgactacagagttaagaaggagatagtttataaagatgtagtgagattttttgattcattaagagaagtgatggatgatctaggtcatgaacttaaattaaatgaaacgattataagtagcaagatgttcatatatagcaaaagaatctattatgatgggagaattcttcctcaagctctaaaagcattatctagatgtgtcttctggtcagagacagtaatagacgaaacaagatcagcatcttcaaatttggcaacatcatttgcaaaagcaattgagaatggttattcacctgttctaggatatgcatgctcaatttttaagaatattcaacaactatatattgcccttgggatgaatatcaatccaactataacacagaatatcagagatcagtattttaggaatccaaattggatgcaatatgcctctttaatacctgctagtgttgggggattcaattacatggccatgtcaagatgttttgtaaggaatattggtgatccatcagttgccgcattggctgatattaaaagatttattaaggcgaatctattagaccgaagtgttctttataggattatgaatcaagaaccaggtgagtcatcttttttggactgggcttcagatccatattcatgcaatttaccacaatctcaaaatataaccaccatgataaaaaatataacagcaaggaatgtattacaagattcaccaaatccattattatctggattattcacaaatacaatgatagaagaagatgaagaattagctgagttcctgatggacaggaaggtaattctccctagagttgcacatgatattctagataattctctcacaggaattagaaatgccatagctggaatgttagatacgacaaaatcactaattcgggttggcataaatagaggaggactgacatatagtttgttgaggaaaatcagtaattacgatctagtacaatatgaaacactaagtaggactttgcgactaattgtaagtgataaaatcaagtatgaagatatgtgttcggtagaccttgccatagcattgcgacaaaagatgtggattcatttatcaggaggaaggatgataagtggacttgaaacgcctgacccattagaattactatctggggtagtaataacaggatcagaacattgtaaaatatgttattcttcagatggcacaaacccatatacttggatgtatttacccggtaatatcaaaataggatcagcagaaacaggtatatcgtcattaagagttccttattttggatcagtcactgatgaaagatctgaagcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttaacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagatactaatcttatttatcaacaaataatgttaacaggattaagtgttttcgaatatttatttagattaaaagaaaccacaggacacaaccctatagttatgcatctgcacatagaagatgagtgttgtattaaagaaagttttaatgatgaacatattaatccagagtctacattagaattaattcgatatcctgaaagtaatgaatttatttatgataaagacccactcaaagatgtggacttatcaaaacttatggttattaaagaccattcttacacaattgatatgaattattgggatgatactgacatcatacatgcaatttcaatatgtactgcaattacaatagcagatactatgtcacaattagatcgagataatttaaaagagataatagttattgcaaatgatgatgatattaatagcttaatcactgaatttttgactcttgacatacttgtatttctcaagacatttggtggattattagtaaatcaatttgcatacactctttatagtctaaaaatagaaggtagggatctcatttgggattatataatgagaacactgagagatacttcccattcaatattaaaagtattatctaatgcattatctcatcctaaagtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatatacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgcggtattagtccacctgaggtaattgatgattgggatccggtagaagatgaaaatatgctggataacattgtcaaaactataaatgataactgtaataaagataataaagggaataaaattaacaatttctggggactagcacttaagaactatcaagtccttaaaatcagatctataacaagtgattctgatgataatgatagactagatgctaatacaagtggtttgacacttcctcaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggtaaaaaattaggaaatgtgacacagattcttaacagggtaaaagtactgttcaatgggaatcctaattcaacatggataggaaatatggaatgtgagagcttaatatggagtgaattaaatgataagtccattggattagtacattgtgatatggaaggagctatcggtaaatcagaagaaactgttctacatgaacattatagtgttataagaattacatacttgattggggatgatgatgttgttttagtttccaaaattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaggaataatgaatggttacatcatgaaatcaaagaaggagaaagagattatggaatcatgagaccatatcatatggcactacaaatctttggatttcaaatcaatttaaatcatctggcgaaagaatttttatcaaccccagatctgactaatatcaacaatataatccaaagttttcagcgaacaataaaggatgttttatttgaatggattaatataactcatgatgataagagacataaattaggcggaagatataacatattcccactgaaaaataagggaaagttaagactgctatcgagaagactagtattaagttggatttcattatcattatcgactcgattacttacaggtcgctttcctgatgaaaaatttgaacatagagcacagactggatatgtatcattagctgatactgatttagaatcattaaagttattgtcgaaaaacatcattaagaattacagagagtgtataggatcaatatcatattggtttctaaccaaagaagttaaaatacttatgaaattgatcggtggtgctaaattattaggaattcccagacaatataaagaacccgaagaccagttattagaaaactacaatcaacatgatgaatttgatatcgattaaaacataaatacaatgaagatatatcctaacctttatctttaagcctaggaatagacaaaaagtaagaaaaacatgtaatatatatataccaaacagagttcttctcttgtttggtgggtcggcatggcatctccacctcctcgcggtccggacctgggcatccgaaggaggacgcacgtccactcggatggctaagggagagcctgcagtagcataaccccttggggcctctaaacgggtcttgaggggttttttgctgaaaggaggaactatatacgcgtcgacgggccccgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcggggcccgtcgacgcgttaatacgactcactatagg

SEQ ID NO: 8 - DNA, 19167 bp, synthetic construct
  misc_feature 1..19167: note = recombinant polynucleotide
  source 1..19167: mol_type = other DNA
accaaacaagagaagaaacttgtctgggaatataaatttaactttaaattaacttaggattaaagacattgactagaaggtcaagaaaagggaactctataatttcaaaaatgttgagcctatttgatacatttaatgcacgtaggcaagaaaacataacaaaatcagccggtggagctatcattcctggacagaaaaatactgtctctatattcgcccttggaccgacaataactgatgataatgagaaaatgacattagctcttctatttctatctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaagatctaaaacggcaaaagtatggaggatttgtggttaagacgagagagatgatatatgaaaagacaactgattggatatttggaagtgacctggattatgatcaggaaactatgttgcagaacggcaggaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagaccagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgccgaagctcaaatgagctcaacactggaagatgaacttggagtgacacacgaatctaaagaaagcttgaagagacatataaggaacataaacagttcagagacatctttccacaaaccgacaggtggatcagccatagagatggcaatagatgaagagccagaacaattcgaacatagagcagatcaagaacaaaatggagaacctcaatcatccataattcaatatgcctgggcagaaggaaatagaagcgatgatcagactgagcaagctacagaatctgacaatatcaagaccgaacaacaaaacatcagagacagactaaacaagagactcaacgacaagaagaaacaaagcagtcaaccacccactaatcccacaaacagaacaaaccaggacgaaatagatgatctgtttaacgcatttggaagcaactaatcgaatcaacattttaatctaaatcaataataaataagaaaaacttaggattaaagaatcctatcataccggaatatagggtggtaaatttagagtctgcttgaaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacttatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctggaggaatccccagaagcatcacagattctaaaaatggaacccaaaacacggaggatattgatctcaatgaaattagaaagatggataaggactctattgaggggaaaatgcgacaatctgcaaatgttccaagcgagatatcaggaagtgatgacatatttacaacagaacaaagtagaaacagtgatcatggaagaagcctggaatctatcagtacacctgatacaagatcaataagtgttgttactgctgcaacaccagatgatgaagaagaaatactaatgaaaaatagtaggacaaagaaaagttcttcaacacatcaagaagatgacaaaagaattaaaaaagggggaaaagggaaagactggtttaagaaatcaaaagataccgacaaccagataccaacatcagactacagatccacatcaaaagggcagaagaaaatctcaaagacaacaaccaccaacaccgacacaaaggggcaaacagaaatacagacagaatcatcagaaacacaatcctcatcatggaatctcatcatcgacaacaacaccgaccggaacgaacagacaagcacaactcctccaacaacaacttccagatcaacttatacaaaagaatcgatccgaacaaactctgaatccaaacccaagacacaaaagacaaatggaaaggaaaggaaggatacagaagagagcaatcgatttacagagagggcaattactctattgcagaatcttggtgtaattcaatccacatcaaaactagatttatatcaagacaaacgagttgtatgtgtagcaaatgtactaaacaatgtagatactgcatcaaagatagatttcctggcaggattagtcataggggtttcaatggacaacgacacaaaattaacacagatacaaaatgaaatgctaaacctcaaagcagatctaaagaaaatggacgaatcacatagaagattgatagaaaatcaaagagaacaactgtcattgatcacgtcactaatttcaaatctcaaaattatgactgagagaggaggaaagaaagaccaaaatgaatccaatgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatattaagttcatacaatgagtcaaatgcaacaagactaatacccaaaaaagtgagcagtacaatgagatcactagttgcagtcatcaacaacagcaatctctcacaaagcacaaaacaatcatacataaacgaactcaaacgttgcaaaaatgatgaagaagtatctgaattaatggacatgttcaatgaagatgtcaacaattgccaatgatccaacaaagaaacgacaccgaacaaacagacaagaaacaacagtagatcaaaacctgtcaacacacacaaaatcaagcagaatgaaacaacagatacttaagaatatacaaataagaaaaacttaggattaaagagccatggtcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagaaatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagtaatatagctaagcttacgcgtttaagaatatacaaataagaaaaacttaggattaaagaataaattaatccttgtccaaaatgagtataactaactctgcaatatacacattcccagaatcatcattctctgaaaatggtcatatagaaccattaccactcaaagtcaatgaacagaggaaagcagtaccccacattagagttgccaagatcggaaatccaccaaaacacggatcccggtatttagatgtcttcttactcggcttcttcgagatggaacgaatcaaagacaaatacgggagtgtgaatgatctcgacagtgacccgagttacaaagtttgtggctctggatcattaccaatcggattggctaagtacactgggaatgaccaggaattgttacaagccgcaaccaaactggatatagaagtgagaagaacagtcaaagcgaaagagatggttgtttacacggtacaaaatataaaaccagaactgtacccgtggtccaatagactaagaaaaggaatgctgttcgatgccaacaaagttgctcttgctcctcaatgtcttccactagataggagcataaaatttagagtaatcttcgtgaattgtacggcaattggatcaataaccttgttcaaaattcctaagtcaatggcatcactatctctacccaacacaatatcaatcaatctgcaggtacacataaaaacaggggttcagactgattctaaagggatagttcaaattttggatgagaaaggcgaaaaatcactgaatttcatggtccatctcggattgatcaaaagaaaagtaggcagaatgtactctgttgaatactgtaaacagaaaatcgagaaaatgagattgatattttctttaggactagttggaggaatcagtcttcatgtcaatgcaactgggtccatatcaaaaacactagcaagtcagctggtattcaaaagagagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctattttagtccggacgtatctattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatatagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtacttggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacaaaagggaaatataaaaaacttaggagcaaagcgtgctcgggaaatggacactgaatctaacaatggcactgtatctgacatactctatcctgagtgtcaccttaactctcctatcgttaaaggtaaaatagcacaattacacactattatgagtctacctcagccttatgatatggatgacgactcaatactagttatcactagacagaaaataaaacttaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcgaaagttcacacaacctataaatcagataaatggtataatccattcaaaacatggtttactatcaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattgtttccaattatgggagaaaagacatttgatgtgatatcgttattagaaccacttgcattatccttaattcaaactcatgatcctgttaaacaactaagaggagcttttttaaatcatgtgttatccgagatggaattaatatttgaatctagagaatcgattaaggaatttctgagtgtagattacattgataaaattttagatatatttaataagtctacaatagatgaaatagcagagattttctctttttttagaacatttgggcatcctccattagaagctagtattgcagcagaaaaggttagaaaatatatgtatattggaaaacaattaaaatttgacactattaataaatgtcatgctatcttctgtacaataataattaacggatatagagagaggcatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaggatttgacaatttatatgaaagataaagcattatctccaaaaaaatcaaattgggacacagtttatcctgcatctaatttactgtaccgtactaacgcatccaacgaatcacgaagattagttgaagtatttatagcagatagtaaatttgatcctcatcagatattggattatgtagaatctggggactggttagatgatccagaatttaatatttcttatagtcttaaagaaaaagagatcaaacaggaaggtagactctttgcaaaaatgacatacaaaatgagagctacacaagttttatcagagaccctacttgcaaataacataggaaaattctttcaagaaaatgggatggtgaagggagagattgaattacttaagagattaacaaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttggattaaataaattgtttaattggttacaccctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcaatacatctagcagctgttagaataggcgtgagggtgactgcaatggttcaaggagacaatcaagctatagctgtaaccacaagagtacccaacaattatgactacagagttaagaaggagatagtttataaagatgtagtgagattttttgattcattaagagaagtgatggatgatctaggtcatgaacttaaattaaatgaaacgattataagtagcaagatgttcatatatagcaaaagaatctattatgatgggagaattcttcctcaagctctaaaagcattatctagatgtgtcttctggtcagagacagtaatagacgaaacaagatcagcatcttcaaatttggcaacatcatttgcaaaagcaattgagaatggttattcacctgttctaggatatgcatgctcaatttttaagaatattcaacaactatatattgcccttgggatgaatatcaatccaactataacacagaatatcagagatcagtattttaggaatccaaattggatgcaatatgcctctttaatacctgctagtgttgggggattcaattacatggccatgtcaagatgttttgtaaggaatattggtgatccatcagttgccgcattggctgatattaaaagatttattaaggcgaatctattagaccgaagtgttctttataggattatgaatcaagaaccaggtgagtcatcttttttggactgggcttcagatccatattcatgcaatttaccacaatctcaaaatataaccaccatgataaaaaatataacagcaaggaatgtattacaagattcaccaaatccattattatctggattattcacaaatacaatgatagaagaagatgaagaattagctgagttcctgatggacaggaaggtaattctccctagagttgcacatgatattctagataattctctcacaggaattagaaatgccatagctggaatgttagatacgacaaaatcactaattcgggttggcataaatagaggaggactgacatatagtttgttgaggaaaatcagtaattacgatctagtacaatatgaaacactaagtaggactttgcgactaattgtaagtgataaaatcaagtatgaagatatgtgttcggtagaccttgccatagcattgcgacaaaagatgtggattcatttatcaggaggaaggatgataagtggacttgaaacgcctgacccattagaattactatctggggtagtaataacaggatcagaacattgtaaaatatgttattcttcagatggcacaaacccatatacttggatgtatttacccggtaatatcaaaataggatcagcagaaacaggtatatcgtcattaagagttccttattttggatcagtcactgatgaaagatctgaagcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttaacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagatactaatcttatttatcaacaaataatgttaacaggattaagtgttttcgaatatttatttagattaaaagaaaccacaggacacaaccctatagttatgcatctgcacatagaagatgagtgttgtattaaagaaagttttaatgatgaacatattaatccagagtctacattagaattaattcgatatcctgaaagtaatgaatttatttatgataaagacccactcaaagatgtggacttatcaaaacttatggttattaaagaccattcttacacaattgatatgaattattgggatgatactgacatcatacatgcaatttcaatatgtactgcaattacaatagcagatactatgtcacaattagatcgagataatttaaaagagataatagttattgcaaatgatgatgatattaatagcttaatcactgaatttttgactcttgacatacttgtatttctcaagacatttggtggattattagtaaatcaatttgcatacactctttatagtctaaaaatagaaggtagggatctcatttgggattatataatgagaacactgagagatacttcccattcaatattaaaagtattatctaatgcattatctcatcctaaagtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatatacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgcggtattagtccacctgaggtaattgatgattgggatccggtagaagatgaaaatatgctggataacattgtcaaaactataaatgataactgtaataaagataataaagggaataaaattaacaatttctggggactagcacttaagaactatcaagtccttaaaatcagatctataacaagtgattctgatgataatgatagactagatgctaatacaagtggtttgacacttcctcaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggtaaaaaattaggaaatgtgacacagattcttaacagggtaaaagtactgttcaatgggaatcctaattcaacatggataggaaatatggaatgtgagagcttaatatggagtgaattaaatgataagtccattggattagtacattgtgatatggaaggagctatcggtaaatcagaagaaactgttctacatgaacattatagtgttataagaattacatacttgattggggatgatgatgttgttttagtttccaaaattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaggaataatgaatggttacatcatgaaatcaaagaaggagaaagagattatggaatcatgagaccatatcatatggcactacaaatctttggatttcaaatcaatttaaatcatctggcgaaagaatttttatcaaccccagatctgactaatatcaacaatataatccaaagttttcagcgaacaataaaggatgttttatttgaatggattaatataactcatgatgataagagacataaattaggcggaagatataacatattcccactgaaaaataagggaaagttaagactgctatcgagaagactagtattaagttggatttcattatcattatcgactcgattacttacaggtcgctttcctgatgaaaaatttgaacatagagcacagactggatatgtatcattagctgatactgatttagaatcattaaagttattgtcgaaaaacatcattaagaattacagagagtgtataggatcaatatcatattggtttctaaccaaagaagttaaaatacttatgaaattgatcggtggtgctaaattattaggaattcccagacaatataaagaacccgaagaccagttattagaaaactacaatcaacatgatgaatttgatatcgattaaaacataaatacaatgaagatatatcctaacctttatctttaagcctaggaatagacaaaaagtaagaaaaacatgtaatatatatataccaaacagagttcttctcttgtttggtgggtcggcatggcatctccacctcctcgcggtccggacctgggcatccgaaggaggacgcacgtccactcggatggctaagggagagcctgcagtagcataaccccttggggcctctaaacgggtcttgaggggttttttgctgaaaggaggaactatatacgcgtcgacgggccccgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcggggcccgtcgacgcgttaatacgactcactatagg

SEQ ID NO: 9 - DNA, 20583 bp, synthetic construct
  misc_feature 1..20583: note = recombinant polynucleotide
  source 1..20583: mol_type = other DNA
accaaacaagagaagaaacttgtctgggaatataaatttaactttaaattaacttaggattaaagacattgactagaaggtcaagaaaagggaactctataatttcaaaaatgttgagcctatttgatacatttaatgcacgtaggcaagaaaacataacaaaatcagccggtggagctatcattcctggacagaaaaatactgtctctatattcgcccttggaccgacaataactgatgataatgagaaaatgacattagctcttctatttctatctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaagatctaaaacggcaaaagtatggaggatttgtggttaagacgagagagatgatatatgaaaagacaactgattggatatttggaagtgacctggattatgatcaggaaactatgttgcagaacggcaggaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagaccagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgccgaagctcaaatgagctcaacactggaagatgaacttggagtgacacacgaatctaaagaaagcttgaagagacatataaggaacataaacagttcagagacatctttccacaaaccgacaggtggatcagccatagagatggcaatagatgaagagccagaacaattcgaacatagagcagatcaagaacaaaatggagaacctcaatcatccataattcaatatgcctgggcagaaggaaatagaagcgatgatcagactgagcaagctacagaatctgacaatatcaagaccgaacaacaaaacatcagagacagactaaacaagagactcaacgacaagaagaaacaaagcagtcaaccacccactaatcccacaaacagaacaaaccaggacgaaatagatgatctgtttaacgcatttggaagcaactaatcgaatcaacattttaatctaaatcaataataaataagaaaaacttaggattaaagaatcctatcataccggaatatagggtggtaaatttagagtctgcttgaaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacttatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctggaggaatccccagaagcatcacagattctaaaaatggaacccaaaacacggaggatattgatctcaatgaaattagaaagatggataaggactctattgaggggaaaatgcgacaatctgcaaatgttccaagcgagatatcaggaagtgatgacatatttacaacagaacaaagtagaaacagtgatcatggaagaagcctggaatctatcagtacacctgatacaagatcaataagtgttgttactgctgcaacaccagatgatgaagaagaaatactaatgaaaaatagtaggacaaagaaaagttcttcaacacatcaagaagatgacaaaagaattaaaaaagggggaaaagggaaagactggtttaagaaatcaaaagataccgacaaccagataccaacatcagactacagatccacatcaaaagggcagaagaaaatctcaaagacaacaaccaccaacaccgacacaaaggggcaaacagaaatacagacagaatcatcagaaacacaatcctcatcatggaatctcatcatcgacaacaacaccgaccggaacgaacagacaagcacaactcctccaacaacaacttccagatcaacttatacaaaagaatcgatccgaacaaactctgaatccaaacccaagacacaaaagacaaatggaaaggaaaggaaggatacagaagagagcaatcgatttacagagagggcaattactctattgcagaatcttggtgtaattcaatccacatcaaaactagatttatatcaagacaaacgagttgtatgtgtagcaaatgtactaaacaatgtagatactgcatcaaagatagatttcctggcaggattagtcataggggtttcaatggacaacgacacaaaattaacacagatacaaaatgaaatgctaaacctcaaagcagatctaaagaaaatggacgaatcacatagaagattgatagaaaatcaaagagaacaactgtcattgatcacgtcactaatttcaaatctcaaaattatgactgagagaggaggaaagaaagaccaaaatgaatccaatgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatattaagttcatacaatgagtcaaatgcaacaagactaatacccaaaaaagtgagcagtacaatgagatcactagttgcagtcatcaacaacagcaatctctcacaaagcacaaaacaatcatacataaacgaactcaaacgttgcaaaaatgatgaagaagtatctgaattaatggacatgttcaatgaagatgtcaacaattgccaatgatccaacaaagaaacgacaccgaacaaacagacaagaaacaacagtagatcaaaacctgtcaacacacacaaaatcaagcagaatgaaacaacagatacttaagaatatacaaataagaaaaacttaggattaaagagccatggtcgtgttcctagtcctactaccgctagtctcttctcagtgtgtaaacctaacaacgagaacacaactaccaccggcgtacaccaattctttcacaagaggagtatattacccggacaaggtgttcagatcctccgtactacattctacacaggacctattcctaccgttcttctctaacgtaacatggttccacgcgatccatgtctctggaacaaacggaacgaagagattcgataacccggtcttgccgttcaacgatggtgtatactttgcgtccaccgagaagtccaacatcatcagaggatggatcttcggaaccaccttggattctaagacccagtccttgctaatcgtcaacaacgcgaccaacgtcgtcatcaaagtctgcgaattccagttctgtaacgacccgttcttgggagtctactaccacaagaacaacaagtcctggatggaatccgagttcagagtctactcttccgcgaacaactgcaccttcgaatatgtatctcagccgttcctaatggacctagagggaaagcagggaaacttcaagaacctaagagagttcgtattcaagaacatcgacggatacttcaagatctactccaagcacactccgatcaacctagttagagatctaccgcaaggattctctgcgctagaaccgttagtagatttgccgatcggaatcaacatcaccagattccagacactactagcgctacacagatcttacctaacgccgggagattcttcttctggatggactgctggtgctgcggcttattatgtaggatacctacagccgagaaccttcctattgaagtacaacgaaaacggaaccatcaccgatgccgtagattgtgctctagatccgctatccgaaacgaagtgcaccctaaagtctttcaccgtcgagaagggaatctaccagacctccaactttagagtacagccgaccgaatccatcgtcagatttccgaacatcacgaacctatgtccgttcggagaagtgttcaacgcgacaagatttgcgtctgtctatgcgtggaacagaaaaagaatcagtaactgcgtcgcggactactccgtcctatacaactctgcctctttctccacgttcaaatgctacggtgtatctccgacaaagctaaacgatctatgcttcaccaacgtctacgcggactccttcgtaatcagaggagatgaagttagacagattgcgccgggacaaactggaaagatcgcggattataactacaagctaccggacgacttcaccggatgtgtaattgcgtggaattcgaacaacctagactccaaagtcggaggaaactacaactacttgtacagactattcagaaagtccaacctaaagccgttcgagagagacatctccaccgaaatctatcaggctggatctacaccgtgtaatggtgtcgaaggattcaactgctacttcccgctacagtcttacggatttcaaccgacaaacggtgtaggatatcagccgtacagagtcgtcgtactatccttcgaactactacatgctccggcgacagtatgtggaccgaaaaagtctaccaacctagtcaagaacaaatgcgtcaactttaacttcaacggactaaccggaaccggtgtcctaaccgaatctaacaagaagtttctaccgttccagcagttcggaagagatatcgcggatacaacagacgctgtcagagatccgcaaaccttggagatcctagatatcacaccgtgttctttcggtggtgtctctgtaattactccgggaacgaacacctccaatcaagtagcggtactataccaggacgtgaactgtacagaagtaccggtagctattcacgcggatcaactaacaccaacttggagagtgtactccaccggatctaacgtattccaaacaagagcgggatgtctaatcggagcggaacacgtaaacaactcctacgaatgtgatatcccgattggagcgggaatctgtgcgtcttaccaaacacaaacaaactctccgagaagagcgagataatatagctaagcttacgcgtttaagaatatacaaataagaaaaacttaggattaaagaataaattaatccttgtccaaaatgagtataactaactctgcaatatacacattcccagaatcatcattctctgaaaatggtcatatagaaccattaccactcaaagtcaatgaacagaggaaagcagtaccccacattagagttgccaagatcggaaatccaccaaaacacggatcccggtatttagatgtcttcttactcggcttcttcgagatggaacgaatcaaagacaaatacgggagtgtgaatgatctcgacagtgacccgagttacaaagtttgtggctctggatcattaccaatcggattggctaagtacactgggaatgaccaggaattgttacaagccgcaaccaaactggatatagaagtgagaagaacagtcaaagcgaaagagatggttgtttacacggtacaaaatataaaaccagaactgtacccgtggtccaatagactaagaaaaggaatgctgttcgatgccaacaaagttgctcttgctcctcaatgtcttccactagataggagcataaaatttagagtaatcttcgtgaattgtacggcaattggatcaataaccttgttcaaaattcctaagtcaatggcatcactatctctacccaacacaatatcaatcaatctgcaggtacacataaaaacaggggttcagactgattctaaagggatagttcaaattttggatgagaaaggcgaaaaatcactgaatttcatggtccatctcggattgatcaaaagaaaagtaggcagaatgtactctgttgaatactgtaaacagaaaatcgagaaaatgagattgatattttctttaggactagttggaggaatcagtcttcatgtcaatgcaactgggtccatatcaaaaacactagcaagtcagctggtattcaaaagagagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctattttagtccggacgtatctattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatatagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtacttggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacaaaagggaaatataaaaaacttaggagcaaagcgtgctcgggaaatggacactgaatctaacaatggcactgtatctgacatactctatcctgagtgtcaccttaactctcctatcgttaaaggtaaaatagcacaattacacactattatgagtctacctcagccttatgatatggatgacgactcaatactagttatcactagacagaaaataaaacttaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcgaaagttcacacaacctataaatcagataaatggtataatccattcaaaacatggtttactatcaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattgtttccaattatgggagaaaagacatttgatgtgatatcgttattagaaccacttgcattatccttaattcaaactcatgatcctgttaaacaactaagaggagcttttttaaatcatgtgttatccgagatggaattaatatttgaatctagagaatcgattaaggaatttctgagtgtagattacattgataaaattttagatatatttaataagtctacaatagatgaaatagcagagattttctctttttttagaacatttgggcatcctccattagaagctagtattgcagcagaaaaggttagaaaatatatgtatattggaaaacaattaaaatttgacactattaataaatgtcatgctatcttctgtacaataataattaacggatatagagagaggcatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaggatttgacaatttatatgaaagataaagcattatctccaaaaaaatcaaattgggacacagtttatcctgcatctaatttactgtaccgtactaacgcatccaacgaatcacgaagattagttgaagtatttatagcagatagtaaatttgatcctcatcagatattggattatgtagaatctggggactggttagatgatccagaatttaatatttcttatagtcttaaagaaaaagagatcaaacaggaaggtagactctttgcaaaaatgacatacaaaatgagagctacacaagttttatcagagaccctacttgcaaataacataggaaaattctttcaagaaaatgggatggtgaagggagagattgaattacttaagagattaacaaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttggattaaataaattgtttaattggttacaccctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcaatacatctagcagctgttagaataggcgtgagggtgactgcaatggttcaaggagacaatcaagctatagctgtaaccacaagagtacccaacaattatgactacagagttaagaaggagatagtttataaagatgtagtgagattttttgattcattaagagaagtgatggatgatctaggtcatgaacttaaattaaatgaaacgattataagtagcaagatgttcatatatagcaaaagaatctattatgatgggagaattcttcctcaagctctaaaagcattatctagatgtgtcttctggtcagagacagtaatagacgaaacaagatcagcatcttcaaatttggcaacatcatttgcaaaagcaattgagaatggttattcacctgttctaggatatgcatgctcaatttttaagaatattcaacaactatatattgcccttgggatgaatatcaatccaactataacacagaatatcagagatcagtattttaggaatccaaattggatgcaatatgcctctttaatacctgctagtgttgggggattcaattacatggccatgtcaagatgttttgtaaggaatattggtgatccatcagttgccgcattggctgatattaaaagatttattaaggcgaatctattagaccgaagtgttctttataggattatgaatcaagaaccaggtgagtcatcttttttggactgggcttcagatccatattcatgcaatttaccacaatctcaaaatataaccaccatgataaaaaatataacagcaaggaatgtattacaagattcaccaaatccattattatctggattattcacaaatacaatgatagaagaagatgaagaattagctgagttcctgatggacaggaaggtaattctccctagagttgcacatgatattctagataattctctcacaggaattagaaatgccatagctggaatgttagatacgacaaaatcactaattcgggttggcataaatagaggaggactgacatatagtttgttgaggaaaatcagtaattacgatctagtacaatatgaaacactaagtaggactttgcgactaattgtaagtgataaaatcaagtatgaagatatgtgttcggtagaccttgccatagcattgcgacaaaagatgtggattcatttatcaggaggaaggatgataagtggacttgaaacgcctgacccattagaattactatctggggtagtaataacaggatcagaacattgtaaaatatgttattcttcagatggcacaaacccatatacttggatgtatttacccggtaatatcaaaataggatcagcagaaacaggtatatcgtcattaagagttccttattttggatcagtcactgatgaaagatctgaagcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttaacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagatactaatcttatttatcaacaaataatgttaacaggattaagtgttttcgaatatttatttagattaaaagaaaccacaggacacaaccctatagttatgcatctgcacatagaagatgagtgttgtattaaagaaagttttaatgatgaacatattaatccagagtctacattagaattaattcgatatcctgaaagtaatgaatttatttatgataaagacccactcaaagatgtggacttatcaaaacttatggttattaaagaccattcttacacaattgatatgaattattgggatgatactgacatcatacatgcaatttcaatatgtactgcaattacaatagcagatactatgtcacaattagatcgagataatttaaaagagataatagttattgcaaatgatgatgatattaatagcttaatcactgaatttttgactcttgacatacttgtatttctcaagacatttggtggattattagtaaatcaatttgcatacactctttatagtctaaaaatagaaggtagggatctcatttgggattatataatgagaacactgagagatacttcccattcaatattaaaagtattatctaatgcattatctcatcctaaagtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatatacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgcggtattagtccacctgaggtaattgatgattgggatccggtagaagatgaaaatatgctggataacattgtcaaaactataaatgataactgtaataaagataataaagggaataaaattaacaatttctggggactagcacttaagaactatcaagtccttaaaatcagatctataacaagtgattctgatgataatgatagactagatgctaatacaagtggtttgacacttcctcaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggtaaaaaattaggaaatgtgacacagattcttaacagggtaaaagtactgttcaatgggaatcctaattcaacatggataggaaatatggaatgtgagagcttaatatggagtgaattaaatgataagtccattggattagtacattgtgatatggaaggagctatcggtaaatcagaagaaactgttctacatgaacattatagtgttataagaattacatacttgattggggatgatgatgttgttttagtttccaaaattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaggaataatgaatggttacatcatgaaatcaaagaaggagaaagagattatggaatcatgagaccatatcatatggcactacaaatctttggatttcaaatcaatttaaatcatctggcgaaagaatttttatcaaccccagatctgactaatatcaacaatataatccaaagttttcagcgaacaataaaggatgttttatttgaatggattaatataactcatgatgataagagacataaattaggcggaagatataacatattcccactgaaaaataagggaaagttaagactgctatcgagaagactagtattaagttggatttcattatcattatcgactcgattacttacaggtcgctttcctgatgaaaaatttgaacatagagcacagactggatatgtatcattagctgatactgatttagaatcattaaagttattgtcgaaaaacatcattaagaattacagagagtgtataggatcaatatcatattggtttctaaccaaagaagttaaaatacttatgaaattgatcggtggtgctaaattattaggaattcccagacaatataaagaacccgaagaccagttattagaaaactacaatcaacatgatgaatttgatatcgattaaaacataaatacaatgaagatatatcctaacctttatctttaagcctaggaatagacaaaaagtaagaaaaacatgtaatatatatataccaaacagagttcttctcttgtttggtgggtcggcatggcatctccacctcctcgcggtccggacctgggcatccgaaggaggacgcacgtccactcggatggctaagggagagcctgcagtagcataaccccttggggcctctaaacgggtcttgaggggttttttgctgaaaggaggaactatatacgcgtcgacgggccccgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcggggcccgtcgacgcgttaatacgactcactatagg

SEQ ID NO: 10 - DNA, 20583 bp, synthetic construct
  misc_feature 1..20583: note = recombinant polynucleotide
  source 1..20583: mol_type = other DNA
accaaacaagagaagaaacttgtctgggaatataaatttaactttaaattaacttaggattaaagacattgactagaaggtcaagaaaagggaactctataatttcaaaaatgttgagcctatttgatacatttaatgcacgtaggcaagaaaacataacaaaatcagccggtggagctatcattcctggacagaaaaatactgtctctatattcgcccttggaccgacaataactgatgataatgagaaaatgacattagctcttctatttctatctcattcactagataatgagaaacaacatgcacaaagggcagggttcttggtgtctttattgtcaatggcttatgccaatccagagctctacctaacaacaaatggaagtaatgcagatgtcaagtatgtcatatacatgattgagaaagatctaaaacggcaaaagtatggaggatttgtggttaagacgagagagatgatatatgaaaagacaactgattggatatttggaagtgacctggattatgatcaggaaactatgttgcagaacggcaggaacaattcaacaattgaagaccttgtccacacatttgggtatccatcatgtttaggagctcttataatacagatctggatagttctggtcaaagctatcactagtatctcagggttaagaaaaggctttttcacccgattggaagctttcagacaagatggaacagtgcaggcagggctggtattgagcggtgacacagtggatcagattgggtcaatcatgcggtctcaacagagcttggtaactcttatggttgaaacattaataacaatgaataccagcagaaatgacctcacaaccatagaaaagaatatacaaattgttggcaactacataagagatgcaggtctcgcttcattcttcaatacaatcagatatggaattgagaccagaatggcagctttgactctatccactctcagaccagatatcaatagattaaaagctttgatggaactgtatttatcaaagggaccacgcgctcctttcatctgtatcctcagagatcctatacatggtgagttcgcaccaggcaactatcctgccatatggagctatgcaatgggggtggcagttgtacaaaatagagccatgcaacagtatgtgacgggaagatcatatctagacattgatatgttccagctaggacaagcagtagcacgtgatgccgaagctcaaatgagctcaacactggaagatgaacttggagtgacacacgaatctaaagaaagcttgaagagacatataaggaacataaacagttcagagacatctttccacaaaccgacaggtggatcagccatagagatggcaatagatgaagagccagaacaattcgaacatagagcagatcaagaacaaaatggagaacctcaatcatccataattcaatatgcctgggcagaaggaaatagaagcgatgatcagactgagcaagctacagaatctgacaatatcaagaccgaacaacaaaacatcagagacagactaaacaagagactcaacgacaagaagaaacaaagcagtcaaccacccactaatcccacaaacagaacaaaccaggacgaaatagatgatctgtttaacgcatttggaagcaactaatcgaatcaacattttaatctaaatcaataataaataagaaaaacttaggattaaagaatcctatcataccggaatatagggtggtaaatttagagtctgcttgaaactcaatcaatagagagttgatggaaagcgatgctaaaaactatcaaatcatggattcttgggaagaggaatcaagagataaatcaactaatatctcctcggccctcaacatcattgaattcatactcagcaccgacccccaagaagacttatcggaaaacgacacaatcaacacaagaacccagcaactcagtgccaccatctgtcaaccagaaatcaaaccaacagaaacaagtgagaaagatagtggatcaactgacaaaaatagacagtctgggtcatcacacgaatgtacaacagaagcaaaagatagaaatattgatcaggaaactgtacagagaggacctgggagaagaagcagctcagatagtagagctgagactgtggtctctggaggaatccccagaagcatcacagattctaaaaatggaacccaaaacacggaggatattgatctcaatgaaattagaaagatggataaggactctattgaggggaaaatgcgacaatctgcaaatgttccaagcgagatatcaggaagtgatgacatatttacaacagaacaaagtagaaacagtgatcatggaagaagcctggaatctatcagtacacctgatacaagatcaataagtgttgttactgctgcaacaccagatgatgaagaagaaatactaatgaaaaatagtaggacaaagaaaagttcttcaacacatcaagaagatgacaaaagaattaaaaaagggggaaaagggaaagactggtttaagaaatcaaaagataccgacaaccagataccaacatcagactacagatccacatcaaaagggcagaagaaaatctcaaagacaacaaccaccaacaccgacacaaaggggcaaacagaaatacagacagaatcatcagaaacacaatcctcatcatggaatctcatcatcgacaacaacaccgaccggaacgaacagacaagcacaactcctccaacaacaacttccagatcaacttatacaaaagaatcgatccgaacaaactctgaatccaaacccaagacacaaaagacaaatggaaaggaaaggaaggatacagaagagagcaatcgatttacagagagggcaattactctattgcagaatcttggtgtaattcaatccacatcaaaactagatttatatcaagacaaacgagttgtatgtgtagcaaatgtactaaacaatgtagatactgcatcaaagatagatttcctggcaggattagtcataggggtttcaatggacaacgacacaaaattaacacagatacaaaatgaaatgctaaacctcaaagcagatctaaagaaaatggacgaatcacatagaagattgatagaaaatcaaagagaacaactgtcattgatcacgtcactaatttcaaatctcaaaattatgactgagagaggaggaaagaaagaccaaaatgaatccaatgagagagtatccatgatcaaaacaaaattgaaagaagaaaagatcaagaagaccaggtttgacccacttatggaggcacaaggcattgacaagaatatacccgatctatatcgacatgcaggagatacactagagaacgatgtacaagttaaatcagagatattaagttcatacaatgagtcaaatgcaacaagactaatacccaaaaaagtgagcagtacaatgagatcactagttgcagtcatcaacaacagcaatctctcacaaagcacaaaacaatcatacataaacgaactcaaacgttgcaaaaatgatgaagaagtatctgaattaatggacatgttcaatgaagatgtcaacaattgccaatgatccaacaaagaaacgacaccgaacaaacagacaagaaacaacagtagatcaaaacctgtcaacacacacaaaatcaagcagaatgaaacaacagatacttaagaatatacaaataagaaaaacttaggattaaagagccatggtcgtgtttctggtgctgctgcctctggtgagctcccagtgcgtgaacctgaccacaaggacccagctgccccctgcctataccaattccttcacacggggcgtgtactatcccgacaaggtgttccggagcagcgtgctgcactccacacaggatctgtttctgcctttcttttctaacgtgacctggttccacgccatccacgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaaaatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgcaggagcagcagcctactatgtgggctatctgcagcccaggaccttcctgctgaagtacaacgagaatggcaccatcacagacgccgtggattgcgccctggatcccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaacccgcttcgccagcgtgtacgcctggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagaaatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggactgaccggaacaggcgtgctgaccgagtccaacaagaagttcctgccttttcagcagttcggcagggacatcgcagataccacagacgccgtgcgcgaccctcagaccctggagatcctggacatcacaccatgctccttcggcggcgtgtctgtgatcacaccaggcaccaatacaagcaaccaggtggccgtgctgtatcaggacgtgaattgtaccgaggtgccagtggcaatccacgcagatcagctgacccctacatggcgggtgtactctaccggcagcaacgtgttccagacaagagccggatgcctgatcggagcagagcacgtgaacaatagctatgagtgcgacatccctatcggcgccggcatctgtgcctcctaccagacccagacaaactccccaaggagagcacggtaatatagctaagcttacgcgtttaagaatatacaaataagaaaaacttaggattaaagaataaattaatccttgtccaaaatgagtataactaactctgcaatatacacattcccagaatcatcattctctgaaaatggtcatatagaaccattaccactcaaagtcaatgaacagaggaaagcagtaccccacattagagttgccaagatcggaaatccaccaaaacacggatcccggtatttagatgtcttcttactcggcttcttcgagatggaacgaatcaaagacaaatacgggagtgtgaatgatctcgacagtgacccgagttacaaagtttgtggctctggatcattaccaatcggattggctaagtacactgggaatgaccaggaattgttacaagccgcaaccaaactggatatagaagtgagaagaacagtcaaagcgaaagagatggttgtttacacggtacaaaatataaaaccagaactgtacccgtggtccaatagactaagaaaaggaatgctgttcgatgccaacaaagttgctcttgctcctcaatgtcttccactagataggagcataaaatttagagtaatcttcgtgaattgtacggcaattggatcaataaccttgttcaaaattcctaagtcaatggcatcactatctctacccaacacaatatcaatcaatctgcaggtacacataaaaacaggggttcagactgattctaaagggatagttcaaattttggatgagaaaggcgaaaaatcactgaatttcatggtccatctcggattgatcaaaagaaaagtaggcagaatgtactctgttgaatactgtaaacagaaaatcgagaaaatgagattgatattttctttaggactagttggaggaatcagtcttcatgtcaatgcaactgggtccatatcaaaaacactagcaagtcagctggtattcaaaagagagatttgttatcctttaatggatctaaatccgcatctcaatctagttatctgggcttcatcagtagagattacaagagtggatgcaattttccaaccttctttacctggcgagttcagatactatcctaatattattgcaaaaggagttgggaaaatcaaacaatggaactagtaatctctattttagtccggacgtatctattaagccgaagcaaataaaggataatcaaaaacttaggacaaaagaggtcaataccaacaactattagcagtcacactcgcaagaataagagagaagggaccaaaaaagtcaaataggagaaatcaaaacaaaaggtacagaacaccagaacaacaaaatcaaaacatccaactcactcaaaacaaaaattccaaaagagaccggcaacacaacaagcactgaacacaatgccaacttcaatactgctaattattacaaccatgatcatggcatctttctgccaaatagatatcacaaaactacagcacgtaggtgtattggtcaacagtcccaaagggatgaagatatcacaaaactttgaaacaagatatctaattttgagcctcataccaaaaatagaagactctaactcttgtggtgaccaacagatcaagcaatacaagaagttattggatagactgatcatccctttatatgatggattaagattacagaaagatgtgatagtaaccaatcaagaatccaatgaaaacactgatcccagaacaaaacgattctttggaggggtaattggaaccattgctctgggagtagcaacctcagcacaaattacagcggcagttgctctggttgaagccaagcaggcaagatcagacatcgaaaaactcaaagaagcaattagggacacaaacaaagcagtgcagtcagttcagagctccataggaaatttaatagtagcaattaaatcagtccaggattatgttaacaaagaaatcgtgccatcgattgcgaggctaggttgtgaagcagcaggacttcaattaggaattgcattaacacagcattactcagaattaacaaacatatttggtgataacataggatcgttacaagaaaaaggaataaaattacaaggtatagcatcattataccgcacaaatatcacagaaatattcacaacatcaacagttgataaatatgatatctatgatctgttatttacagaatcaataaaggtgagagttatagatgttgacttgaatgattactcaatcaccctccaagtcagactccctttattaactaggctgctgaacactcagatctacaaagtagattccatatcatataacatccaaaacagagaatggtatatccctcttcccagccatatcatgacgaaaggggcatttctaggtggagcagacgtcaaagaatgtatagaagcattcagcagctatatatgcccttctgatccaggatttgtattaaaccatgaaatagagagctgcttatcaggaaacatatcccaatgtccaagaacaacggtcacatcagacattgttccaagatatgcatttgtcaatggaggagtggttgcaaactgtataacaaccacctgtacatgcaacggaattggtaatagaatcaatcaaccacctgatcaaggagtaaaaattataacacataaagaatgtagtacaataggtatcaacggaatgctgttcaatacaaataaagaaggaactcttgcattctatacaccaaatgatataacactaaacaattctgttgcacttgatccaattgacatatcaatcgagctcaacaaggccaaatcagatctagaagaatcaaaagaatggataagaaggtcaaatcaaaaactagattctattggaaattggcatcaatctagcactacaatcataattattttgataatgatcattatattgtttataattaatataacgataattacaattgcaattaagtattacagaattcaaaagagaaatcgagtggatcaaaatgacaagccatatgtactaacaaacaaataacatatctacagatcattagatattaaaattataaaaaacttaggagtaaagttacgcaatccaactctactcatataattgaggaaggacccaatagacaaatccaaattcgagatggaatactggaagcataccaatcacggaaaggatgctggtaatgagctggagacgtctatggctactcatggcaacaagctcactaataagataatatacatattatggacaataatcctggtgttattatcaatagtcttcatcatagtgctaattaattccatcaaaagtgaaaaggcccacgaatcattgctgcaagacataaataatgagtttatggaaattacagaaaagatccaaatggcatcggataataccaatgatctaatacagtcaggagtgaatacaaggcttcttacaattcagagtcatgtccagaattacataccaatatcattgacacaacagatgtcagatcttaggaaattcattagtgaaattacaattagaaatgataatcaagaagtgctgccacaaagaataacacatgatgtaggtataaaacctttaaatccagatgatttttggagatgcacgtctggtcttccatctttaatgaaaactccaaaaataaggttaatgccagggccgggattattagctatgccaacgactgttgatggctgtgttagaactccgtctttagttataaatgatctgatttatgcttatacctcaaatctaattactcgaggttgtcaggatataggaaaatcatatcaagtcttacagatagggataataactgtaaactcagacttggtacctgacttaaatcctaggatctctcatacctttaacataaatgacaataggaagtcatgttctctagcactcctaaatatagatgtatatcaactgtgttcaactcccaaagttgatgaaagatcagattatgcatcatcaggcatagaagatattgtacttgatattgtcaattatgatggttcaatctcaacaacaagatttaagaataataacataagctttgatcaaccatatgctgcactatacccatctgttggaccagggatatactacaaaggcaaaataatatttctcgggtatggaggtcttgaacatccaataaatgagaatgtaatctgcaacacaactgggtgccccgggaaaacacagagagactgtaatcaagcatctcatagtacttggttttcagataggaggatggtcaactccatcattgttgttgacaaaggcttaaactcaattccaaaattgaaagtatggacgatatctatgcgacaaaattactgggggtcagaaggaaggttacttctactaggtaacaagatctatatatatacaagatctacaagttggcatagcaagttacaattaggaataattgatattactgattacagtgatataaggataaaatggacatggcataatgtgctatcaagaccaggaaacaatgaatgtccatggggacattcatgtccagatggatgtataacaggagtatatactgatgcatatccactcaatcccacagggagcattgtgtcatctgtcatattagactcacaaaaatcgagagtgaacccagtcataacttactcaacagcaaccgaaagagtaaacgagctggccatcctaaacagaacactctcagctggatatacaacaacaagctgcattacacactataacaaaggatattgttttcatatagtagaaataaatcataaaagcttaaacacatttcaacccatgttgttcaaaacagagattccaaaaagctgcagttaatcataattaaccataatatgcatcaatctatctataatacaagtatatgataagtaatcagcaatcagacaatagacaaaagggaaatataaaaaacttaggagcaaagcgtgctcgggaaatggacactgaatctaacaatggcactgtatctgacatactctatcctgagtgtcaccttaactctcctatcgttaaaggtaaaatagcacaattacacactattatgagtctacctcagccttatgatatggatgacgactcaatactagttatcactagacagaaaataaaacttaataaattggataaaagacaacgatctattagaagattaaaattaatattaactgaaaaagtgaatgacttaggaaaatacacatttatcagatatccagaaatgtcaaaagaaatgttcaaattatatatacctggtattaacagtaaagtgactgaattattacttaaagcagatagaacatatagtcaaatgactgatggattaagagatctatggattaatgtgctatcaaaattagcctcaaaaaatgatggaagcaattatgatcttaatgaagaaattaataatatatcgaaagttcacacaacctataaatcagataaatggtataatccattcaaaacatggtttactatcaagtatgatatgagaagattacaaaaagctcgaaatgagatcacttttaatgttgggaaggattataacttgttagaagaccagaagaatttcttattgatacatccagaattggttttgatattagataaacaaaactataatggttatctaattactcctgaattagtattgatgtattgtgacgtagtcgaaggccgatggaatataagtgcatgtgctaagttagatccaaaattacaatctatgtatcagaaaggtaataacctgtgggaagtgatagataaattgtttccaattatgggagaaaagacatttgatgtgatatcgttattagaaccacttgcattatccttaattcaaactcatgatcctgttaaacaactaagaggagcttttttaaatcatgtgttatccgagatggaattaatatttgaatctagagaatcgattaaggaatttctgagtgtagattacattgataaaattttagatatatttaataagtctacaatagatgaaatagcagagattttctctttttttagaacatttgggcatcctccattagaagctagtattgcagcagaaaaggttagaaaatatatgtatattggaaaacaattaaaatttgacactattaataaatgtcatgctatcttctgtacaataataattaacggatatagagagaggcatggtggacagtggcctcctgtgacattacctgatcatgcacacgaattcatcataaatgcttacggttcaaactctgcgatatcatatgaaaatgctgttgattattaccagagctttataggaataaaattcaataaattcatagagcctcagttagatgaggatttgacaatttatatgaaagataaagcattatctccaaaaaaatcaaattgggacacagtttatcctgcatctaatttactgtaccgtactaacgcatccaacgaatcacgaagattagttgaagtatttatagcagatagtaaatttgatcctcatcagatattggattatgtagaatctggggactggttagatgatccagaatttaatatttcttatagtcttaaagaaaaagagatcaaacaggaaggtagactctttgcaaaaatgacatacaaaatgagagctacacaagttttatcagagaccctacttgcaaataacataggaaaattctttcaagaaaatgggatggtgaagggagagattgaattacttaagagattaacaaccatatcaatatcaggagttccacggtataatgaagtgtacaataattctaaaagccatacagatgaccttaaaacctacaataaaataagtaatcttaatttgtcttctaatcagaaatcaaagaaatttgaattcaagtcaacggatatctacaatgatggatacgagactgtgagctgtttcctaacaacagatctcaaaaaatactgtcttaattggagatatgaatcaacagctctatttggagaaacttgcaaccaaatatttggattaaataaattgtttaattggttacaccctcgtcttgaaggaagtacaatctatgtaggtgatccttactgtcctccatcagataaagaacatatatcattagaggatcaccctgattctggtttttacgttcataacccaagagggggtatagaaggattttgtcaaaaattatggacactcatatctataagtgcaatacatctagcagctgttagaataggcgtgagggtgactgcaatggttcaaggagacaatcaagctatagctgtaaccacaagagtacccaacaattatgactacagagttaagaaggagatagtttataaagatgtagtgagattttttgattcattaagagaagtgatggatgatctaggtcatgaacttaaattaaatgaaacgattataagtagcaagatgttcatatatagcaaaagaatctattatgatgggagaattcttcctcaagctctaaaagcattatctagatgtgtcttctggtcagagacagtaatagacgaaacaagatcagcatcttcaaatttggcaacatcatttgcaaaagcaattgagaatggttattcacctgttctaggatatgcatgctcaatttttaagaatattcaacaactatatattgcccttgggatgaatatcaatccaactataacacagaatatcagagatcagtattttaggaatccaaattggatgcaatatgcctctttaatacctgctagtgttgggggattcaattacatggccatgtcaagatgttttgtaaggaatattggtgatccatcagttgccgcattggctgatattaaaagatttattaaggcgaatctattagaccgaagtgttctttataggattatgaatcaagaaccaggtgagtcatcttttttggactgggcttcagatccatattcatgcaatttaccacaatctcaaaatataaccaccatgataaaaaatataacagcaaggaatgtattacaagattcaccaaatccattattatctggattattcacaaatacaatgatagaagaagatgaagaattagctgagttcctgatggacaggaaggtaattctccctagagttgcacatgatattctagataattctctcacaggaattagaaatgccatagctggaatgttagatacgacaaaatcactaattcgggttggcataaatagaggaggactgacatatagtttgttgaggaaaatcagtaattacgatctagtacaatatgaaacactaagtaggactttgcgactaattgtaagtgataaaatcaagtatgaagatatgtgttcggtagaccttgccatagcattgcgacaaaagatgtggattcatttatcaggaggaaggatgataagtggacttgaaacgcctgacccattagaattactatctggggtagtaataacaggatcagaacattgtaaaatatgttattcttcagatggcacaaacccatatacttggatgtatttacccggtaatatcaaaataggatcagcagaaacaggtatatcgtcattaagagttccttattttggatcagtcactgatgaaagatctgaagcacaattaggatatatcaagaatcttagtaaacctgcaaaagccgcaataagaatagcaatgatatatacatgggcatttggtaatgatgagatatcttggatggaagcctcacagatagcacaaacacgtgcaaattttacactagatagtctcaaaattttaacaccggtagctacatcaacaaatttatcacacagattaaaggatactgcaactcagatgaaattctccagtacatcattgatcagagtcagcagattcataacaatgtccaatgataacatgtctatcaaagaagctaatgaaaccaaagatactaatcttatttatcaacaaataatgttaacaggattaagtgttttcgaatatttatttagattaaaagaaaccacaggacacaaccctatagttatgcatctgcacatagaagatgagtgttgtattaaagaaagttttaatgatgaacatattaatccagagtctacattagaattaattcgatatcctgaaagtaatgaatttatttatgataaagacccactcaaagatgtggacttatcaaaacttatggttattaaagaccattcttacacaattgatatgaattattgggatgatactgacatcatacatgcaatttcaatatgtactgcaattacaatagcagatactatgtcacaattagatcgagataatttaaaagagataatagttattgcaaatgatgatgatattaatagcttaatcactgaatttttgactcttgacatacttgtatttctcaagacatttggtggattattagtaaatcaatttgcatacactctttatagtctaaaaatagaaggtagggatctcatttgggattatataatgagaacactgagagatacttcccattcaatattaaaagtattatctaatgcattatctcatcctaaagtattcaagaggttctgggattgtggagttttaaaccctatttatggtcctaatactgctagtcaagaccagataaaacttgccctatctatatgtgaatattcactagatctatttatgagagaatggttgaatggtgtatcacttgaaatatacatttgtgacagcgatatggaagttgcaaatgataggaaacaagcctttatttctagacacctttcatttgtttgttgtttagcagaaattgcatctttcggacctaacctgttaaacttaacatacttggagagacttgatctattgaaacaatatcttgaattaaatattaaagaagaccctactcttaaatatgtacaaatatctggattattaattaaatcgttcccatcaactgtaacatacgtaagaaagactgcaatcaaatatctaaggattcgcggtattagtccacctgaggtaattgatgattgggatccggtagaagatgaaaatatgctggataacattgtcaaaactataaatgataactgtaataaagataataaagggaataaaattaacaatttctggggactagcacttaagaactatcaagtccttaaaatcagatctataacaagtgattctgatgataatgatagactagatgctaatacaagtggtttgacacttcctcaaggagggaattatctatcgcatcaattgagattattcggaatcaacagcactagttgtctgaaagctcttgagttatcacaaattttaatgaaggaagtcaataaagacaaggacaggctcttcctgggagaaggagcaggagctatgctagcatgttatgatgccacattaggacctgcagttaattattataattcaggtttgaatataacagatgtaattggtcaacgagaattgaaaatatttccttcagaggtatcattagtaggtaaaaaattaggaaatgtgacacagattcttaacagggtaaaagtactgttcaatgggaatcctaattcaacatggataggaaatatggaatgtgagagcttaatatggagtgaattaaatgataagtccattggattagtacattgtgatatggaaggagctatcggtaaatcagaagaaactgttctacatgaacattatagtgttataagaattacatacttgattggggatgatgatgttgttttagtttccaaaattatacctacaatcactccgaattggtctagaatactttatctatataaattatattggaaagatgtaagtataatatcactcaaaacttctaatcctgcatcaacagaattatatctaatttcgaaagatgcatattgtactataatggaacctagtgaaattgttttatcaaaacttaaaagattgtcactcttggaagaaaataatctattaaaatggatcattttatcaaagaagaggaataatgaatggttacatcatgaaatcaaagaaggagaaagagattatggaatcatgagaccatatcatatggcactacaaatctttggatttcaaatcaatttaaatcatctggcgaaagaatttttatcaaccccagatctgactaatatcaacaatataatccaaagttttcagcgaacaataaaggatgttttatttgaatggattaatataactcatgatgataagagacataaattaggcggaagatataacatattcccactgaaaaataagggaaagttaagactgctatcgagaagactagtattaagttggatttcattatcattatcgactcgattacttacaggtcgctttcctgatgaaaaatttgaacatagagcacagactggatatgtatcattagctgatactgatttagaatcattaaagttattgtcgaaaaacatcattaagaattacagagagtgtataggatcaatatcatattggtttctaaccaaagaagttaaaatacttatgaaattgatcggtggtgctaaattattaggaattcccagacaatataaagaacccgaagaccagttattagaaaactacaatcaacatgatgaatttgatatcgattaaaacataaatacaatgaagatatatcctaacctttatctttaagcctaggaatagacaaaaagtaagaaaaacatgtaatatatatataccaaacagagttcttctcttgtttggtgggtcggcatggcatctccacctcctcgcggtccggacctgggcatccgaaggaggacgcacgtccactcggatggctaagggagagcctgcagtagcataaccccttggggcctctaaacgggtcttgaggggttttttgctgaaaggaggaactatatacgcgtcgacgggccccgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcggggcccgtcgacgcgttaatacgactcactatagg

SEQ ID NO: 11 - DNA, 11 bp, synthetic construct
  misc_feature 1..11: note = synthetic polynucleotide
  source 1..11: mol_type = other DNA
aaaaagaaaaa

SEQ ID NO: 12 - DNA, 10 bp, synthetic construct
  misc_feature 1..10: note = synthetic polynucleotide
  source 1..10: mol_type = other DNA
aggattaaag

(19) United States
(12) Patent Application Publication
(10) Pub. No.: US 2025/0064919 A1
(43) Pub. Date: Feb. 27, 2025

(54) A SARS-COV-2 Human Parainfluenza Virus Type 3-Vectored Vaccine

(71) Applicant: Board of Regents, The University of Texas System, Austin, TX (US)

(72) Inventors: Alexander Bukreyev, Galveston, TX (US); Philipp A. Ilinykh, Galveston, TX (US); Sivakumar Periasamy, Galveston, TX (US); Kai Huang, Galveston, TX (US)

(21) Appl. No.: 18/726,909

(22) PCT Filed: Jan. 5, 2023
(86) PCT No.: PCT/US2023/010245

§ 371 Date: Jul. 5, 2024

Related U.S. Application Data
(60) Provisional application No. 63/297,201, filed on Jan. 6, 2022.

(51) Int. Cl. A61K 39/215; A61K 9/00; A61K 39/00; A61P 31/14; C12N 7/00

(52) U.S. Cl. CPC A61K 39/215; A61K 9/0043; A61P 31/14; C12N 7/00; A61K 2039/5256; A61K 2039/543; A61K 2039/545; A61K 2039/575; C12N 2760/18622; C12N 2760/18634; C12N 2770/20034

(57) ABSTRACT

The disclosure is directed to a new human parainfluenza virus (HPIV)/SARS-CoV-2 vaccine or vaccine construct/polynucleotide. Specifically, the HPIV is a Human parainfluenza virus type 3 (HIPV3), and the vaccine construct encoding a SARS-CoV-2 spike protein (S protein), that is a SARS-CoV-2 S protein S1 subunit and/or a SARS-CoV-2 S protein receptor binding domain (RBD), in certain aspects the vaccine or vaccine construct is administered via intranasal administration.

### RELATED APPLICATIONS

This application is an international application claiming priority to U.S. Provisional Application 63/297,201 filed Jan. 6, 2022 which is incorporated herein by reference in its entirety.

### STATEMENT REGARDING FEDERALLY FUNDED RESEARCH

None.

### REFERENCE TO SEQUENCE LISTING

A sequence listing is being submitted electronically with this application. The sequence listing is incorporated herein by reference.

### BACKGROUND

In December 2019, an outbreak of a severe respiratory disease was first reported in the city of Wuhan, Hubei, China. The causative agent of this outbreak was identified as a novel coronavirus named severe acute respiratory syndrome coronavirus-2 (SARS-CoV-2), causing COVID-19 (Zhu et al., 2020). The World Health Organization declared the outbreak a Public Health Emergency of International Concern on Jan. 30, 2020, and a pandemic on Mar. 11, 2020. It spread rapidly around the world, causing more than 261 million cases and 5.2 million deaths as of Nov. 30, 2021 (URL covid19.who.int). Since the last quarter of 2020, variant viruses have emerged in many parts of the world as a result of the high burden of infection and the adaptation of SARS-CoV-2 to human cells under immune pressure (Andreano and Rappuoli, 2021; Subbarao, 2021). While approved SARS-CoV-2 vaccines are being rolled out, many developing countries are still waiting for access to these vaccines. Even with the deployment of safe and effective vaccines, alternative vaccine platforms are needed to address the pandemic (Krause et al., 2021). Furthermore, children and infants, who were considered less susceptible at the beginning of the pandemic, are now representing an important population which requires vaccination (2021).

Neutralizing antibody titers are likely to be an essential correlate of protection against SARS-CoV-2 (Lau et al., 2021). This was further confirmed in the clinical trials of several vaccine candidates (Khoury et al., 2021). The envelope spike(S) glycoprotein of SARS-CoV-2, which enables binding and entry to the host cell, is comprised of two subunits, S1 and S2. The S1 subunit contains the receptor-binding domain (RBD), which is responsible for recognition of the carboxypeptidase angiotensin-converting enzyme 2 (ACE2) receptor on host cells. Being the sole viral antigen that elicits the neutralizing immune response, the S protein serves as a main target for therapeutic antibodies and vaccine design efforts. Its RBD contains numerous conformational B cell epitopes (Walls et al., 2020). RBD-specific antibodies prevent virus attachment to the host cell and were shown to make up most of the virus-neutralizing response during infection (Liu et al., 2020; Ni et al., 2020; Piccoli et al., 2020; Robbiani et al., 2020).

The initial site of SARS-CoV-2 infection is the sinonasal epithelium (Bridges et al., 2021). The pathogenesis of the early stages of COVID-19 is associated with the penetration of the upper respiratory tract by SARS-CoV-2 and the subsequent development of viral infection in tissues of the upper and lower respiratory tracts. The level of lung damage largely determines the severity and outcome of the disease. Therefore, the local immune responses, i.e., the S-specific antibodies on the airway mucosa and T cell immunity, play an important role in prevention of the disease by blocking SARS-CoV-2 infection upon its entry to the respiratory tract. A desirable feature of any COVID-19 vaccine is to stop viral replication in the upper respiratory tract before progression into the lungs. This feature would also strengthen prevention of the interpersonal transmission. Available vaccines against SARS-CoV-2 include those based on mRNA (Baden et al., 2021; Polack et al., 2020), viral vectors expressing the S protein (Logunov et al., 2021; Mercado et al., 2020; van Doremalen et al., 2020; Voysey et al., 2021), inactivated whole virus (Gao et al., 2020; Wang et al., 2020), protein subunit (Keech et al., 2020) or DNA platforms (Smith et al., 2020; Yu et al., 2020), among others. Notably, currently approved vaccines are administered by intramuscular (IM) injection, resulting in robust systemic yet uncertain mucosal immunity. In contrast, intranasal (IN) administration has a great potential to elicit both systemic and local responses with the ease of vaccination, including the production of IgA and stimulation of T and B cells in the nasopharynx-associated lymphoid tissue (Lycke, 2012), that can effectively and immediately eliminate viruses entering the upper respiratory tract. Additionally, IN vaccines provide strategies for improved booster vaccinations after the prime with any of the approved vaccines, since it might also be more effective if sequential vaccinations employ different routes of administration.

### SUMMARY

One solution to the problem of SARS-CoV-2 infection is the design, production, and administration of new vaccines. In certain aspects the new vaccine is a human parainfluenza virus (HPIV) vaccine. Certain embodiments are directed to the use of such a HPIV/SARS-CoV-2 vaccine via intranasal administration.

Respiratory tract vaccination has an advantage of needle-free delivery and induction of mucosal immune response in the portal of SARS-CoV-2 entry. The human parainfluenza virus type 3 vector was used to generate constructs expressing the full spike(S) protein of SARS-CoV-2, its S1 subunit, or the receptor-binding domain (RBD), which were tested in hamsters as single-dose intranasal vaccines. The construct bearing full-length S induced high titers of neutralizing antibodies specific to S protein domains critical to the protein functions. Robust tissue-resident T cell responses in the lungs were also induced, which represent an additional barrier to infection and should be less sensitive than the antibody responses to mutations present in SARS-CoV-2 variants. Following SARS-CoV-2 challenge, animals were protected from the disease and detectable viral replication. Vaccination prevented induction of gene pathways associated with inflammation. These results indicate advantages of respiratory vaccination against COVID-19 and inform the design of mucosal SARS-CoV-2 vaccines.

Certain embodiments are directed to a SARS-CoV-2 vaccine, the vaccine comprising an engineered human parainfluenza virus (HPIV) encoding a SARS-CoV-2 spike protein (S protein) transcriptional cassette. In certain aspects the human parainfluenza virus is human parainfluenza virus type 3 (HIPV3). The SARS-CoV-2 S protein peptide is a SARS-CoV-2 S protein, a SARS-CoV-2 S protein S1 subunit, or a SARS-CoV-2 S protein receptor binding domain (RBD). In certain aspects the SARS-CoV-2 spike protein is at least 80, 85, 90, 95, 98, 99, to 100% identical to 100, 200, 300, 400, 500, 600, 700, 800, 900, 1000, 1100, 1200, up to 1273 consecutive amino acids of the amino acid sequence of SEQ ID NO:2. In certain aspects the transcriptional cassette comprises a HPIV3-specific gene-start and gene-end transcriptional signals upstream and downstream of the SARS-CoV-2 S protein encoding region. The SARS-CoV-2 S protein transcriptional cassette can be incorporated in the P-M intergenic sequence of the engineered HPIV.

Certain embodiments are directed to a human parainfluenza virus (HPIV)/SARS-CoV-2 polynucleotide or construct comprising a HPIV genome having a SARS-CoV-2 transcription cassette insert, wherein the transcription cassette encodes all or part of a SARS-CoV-2 spike protein(S). The terms “transcription cassette,” “expression cassette,” or “cassette” as used herein means a DNA sequence capable of directing expression of a particular nucleotide sequence in an appropriate host cell, comprising a promoter operably linked to the nucleotide sequence which is operably linked to termination signals. The expression of the nucleotide sequence in the expression cassette may be under the control of a constitutive promoter or of an inducible promoter that initiates transcription only when the host cell is exposed to some particular external stimulus. The expression cassette also typically comprises sequences required for proper translation of the nucleotide sequence. The transcriptional cassette comprising the nucleotide sequence of interest may be chimeric. “Chimeric” is used to indicate that a DNA sequence, such as a vector or a gene, is comprised of two or more DNA sequences of distinct origin that are fused together by recombinant DNA techniques resulting in a DNA sequence, which does not occur naturally. A transcriptional cassette, expression cassette or cassette can incorporate numerous nucleotide sequences, promoters, regulatory elements, nucleotide sequences of interest, etc. In certain aspects the transcriptional cassette can have a HPIV3-specific gene-start and gene-end transcriptional signals upstream and downstream, respectively, of the SARS-CoV-2 S protein encoding region. In certain aspects the SARS-CoV-2 S protein transcriptional cassette is incorporated in the P-M intergenic sequence of the engineered HPIV. The construct can have a nucleotide sequence that is at least 80, 90, 95, 98, or 100% identical to SEQ ID NO:3, SEQ ID NO:4, SEQ ID NO:5, SEQ ID NO:6, SEQ ID NO:7, SEQ ID NO:8, SEQ ID NO: 9, or SEQ ID NO:10. In certain aspects the construct has a nucleotide sequence that is at least 98% identical to SEQ ID NO: SEQ ID NO:3, SEQ ID NO:4, SEQ ID NO:5, SEQ ID NO:6, SEQ ID NO:7, SEQ ID NO:8, SEQ ID NO:9, or SEQ ID NO:10. In other aspects the construct has a nucleotide sequence of SEQ ID NO: SEQ ID NO:3, SEQ ID NO:4, SEQ ID NO:5, SEQ ID NO: 6, SEQ ID NO:7, SEQ ID NO:8, SEQ ID NO:9, or SEQ ID NO: 10. The construct can be a DNA or an RNA construct. The DNA construct can be a plasmid or other vector. Certain aspects are directed to a DNA vector encoding the construct described above. Other aspects are directed to a recombinant human parainfluenza virus containing the construct described above as its genome.

Other embodiments are directed to a method of inducing an antigen-specific immune response in a subject, the method comprising administering via intranasal administration to the subject of a vaccine or construct described herein to produce an antigen-specific immune response in the subject. In certain aspects a vaccine composition comprises 10³ to 10⁸ pfu of a HPIV/SARS-CoV-2 vaccine vector.

Certain embodiments are directed to a composition comprising a vaccine or construct described herein formulated for intranasal administration.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 Recovery and characterization of HPIV3-vectored vaccines expressing SARS-CoV-2 S proteins. (A.) Generation of vaccine constructs. The schematic domain organization of the S inserts (full S (see SEQ ID NO:2), S1, RBD1, RBD2) is shown. NTD, N-terminal domain; RBD, receptor-binding domain; RBM, receptor-binding motif; FP, fusion peptide; HR1, heptad repeat 1; HR2, heptad repeat 2; TM, transmembrane domain; CT, cytoplasmic tail. HPIV3 proteins: N, nucleocapsid protein; P, phosphoprotein; M, matrix protein; F, fusion protein; HN, hemagglutinin-neuraminidase; L, RNA polymerase. (B.) LLC-MK2 cell monolayers were infected for 48 hours, lysed, separated on a Western blot and stained for S proteins. Positive control: the recombinant S protein; loading control: actin. (C.) Viral plaques immunostained with rabbit polyclonal antibodies against HPIV3 in LLC-MK2 cells (8 dpi).

FIG. 2. Full S vaccine elicits antibodies neutralizing SARS-CoV-2 and its genetic variants. (A.) Total serum titers of SARS-CoV-2 S-specific IgG measured by ELISA. OD values from pre-bleed serum samples were subtracted from that of the test controls, and titers were determined using a four-parameter logistic curve fit. (B.) Serum neutralization of mNeonGreen-expressing SARS-CoV-2 (WA1/2020) measured by high-throughput screening (HTS). (C.) Serum neutralization of mNeonGreen-expressing SARS-CoV-2 (Kappa and Delta variants) on day 27 post-immunization, HTS format. Neutralization data for WA1/2020 are taken from panel B for comparison. (D.) Serum neutralization of SARS-CoV-2 biological isolates on day 27 postimmunization measured by a standard plaque reduction neutralization assay. Data represent mean±SEM of n=3-10 per group. *p<0.05; **p<0.01; ***p<0.0001 (Two-way ANOVA with multiple comparisons, Fisher's LSD test).

FIG. 3. Vaccine-induced antibodies target a variety of epitopes in the S protein. The S protein linear epitopes targeted by hamster immune sera, were elucidated using peptide microarrays of immobilized 15-mer oligopeptides covering the entire SARS-CoV-2 S sequence with 4 amino acid overlaps. Data for individual animals from HPIV3-wt (1A-10A), HPIV3/full S (11B-20B) and HPIV3/S1 groups (21C, 30C) are presented. Indices show days after immunization (27, 31, 42). Each column represents MFI of a 15-residue peptide matching to the sequence of SARS-CoV-2 S. Shown are mean values of triplicate peptides (for HPIV3/full S group) or single peptide values (for other groups) after subtraction of the corresponding preimmunization sera signals for each individual animal. Dotted lines indicate the arbitrary background level. Identification numbers are shown above the peaks for peptides exceeding background binding. Numbers in black indicate peaks that are present in HPIV3-wt control group serum samples, and numbers in red indicate peaks that are not present in the control group on day 27 post-immunization (i.e., specific for vaccine-raised antibodies). The S protein map (adapted from Stevens et al., 2021) depicts key domains and epitopes for vaccine induced antibodies, corresponding to the coordinates of specific peptides. The definitions for S protein domains are given in FIG. 1.

FIG. 4. Cell-mediated response to vaccination in lungs and spleen. A-D. Cells isolated from tissue samples of uninfected animals at 28 days post vaccination were stimulated with SARS-CoV-2 S-peptide pool and stained for CD4, CD8 and IFNγ markers. A-B. Representative histograms show the frequency of CD4+ and CD8+ T cells (gated from lymphocyte population) and IFNγ-positive CD4+ or CD8+ T cells following treatment with S peptides in lungs (A) or spleen (B). C-D. Frequency and absolute numbers of T cells per 106 total lung cells (C) or spleen cells (D). E. IFNγ secretion by lung and spleen cells at 28 days post vaccination. Cells were isolated from tissue samples of uninfected animals and stimulated with SARSCoV-2 S-peptide pool. The level of IFNγ was determined in supernatants of cultured cells by ELISA. Data represent mean±SEM of 4-5 animals per group. *p<0.05; **p<0.01; ***p<0.001; ****p<0.0001 (One-way ANOVA with multiple comparisons, Fisher's LSD test).

FIG. 5. HPIV3 vaccine expressing full S protein protects hamsters against SARS-CoV-2 infection. Groups of hamsters at ten animals per group were immunized with the indicated vaccine constructs by the intranasal route and challenged with SARS-CoV-2 in 28 days after immunization. Body weight curves are shown (A). On days 3 or 14 post-infection, animals were euthanized to determine viremia (B) and viral load (C) in lungs and nasal turbinates. B, C: each dot corresponds to individual sample. Data represent mean±SEM of n=4-5 per group. *p<0.05; **p<0.01; ***p<0.001; ****p<0.0001 (One-way ANOVA with multiple comparisons, Fisher's LSD test).

FIG. 6. Virus-induced lung pathology in vaccinated hamsters. Representative gross and histological lung images on days 3 (A-E) and 14 (G-K) after infection. Black arrows show congestion and focal consolidation in the lungs of SARS-CoV-2 infected hamsters. A, G: HPIV3-wt group; B, H: HPIV3/full S group; C, I: HPIV3/S1 group; D, J: HPIV3/RBD1 group; E, K: HPIV3/RBD2 group; M: naïve (image for panel M was taken from our previous study, Meyer et al., 2021, for comparison). Magnification: 4×. F, L. Comparative pathology scores on days 3 (F) and 14 (L) after infection calculated based on criteria described in Table 3. Data represent mean±SEM of n=4-5 per group. *p<0.05; **p<0.01; ***p<0.001 (One-way ANOVA with multiple comparisons, Fisher's LSD test).

FIG. 7. Gene expression pattern in vaccinated hamsters at 3 dpi resembles that of naïve animals. RNA seq of lung tissue harvested from naïve animals (unvaccinated, uninfected), or at 3 dpi from HPIV3-wt or HPIV3/full S vaccinated hamsters, was performed. Heat map of the top 300 differentially expressed genes is shown. Hierarchical clustering of genes is shown on the left. The color legend indicates log 2 expression measures.

FIG. 8. Numbers of T and B cells in lungs and spleen at 28 days post vaccination. Cells were isolated from tissue samples of uninfected animals, stained for T (A) or B (B) cell markers and analyzed by flow cytometry. The frequency and total numbers of specific cell populations are shown. Data represent mean±SEM of 4-5 animals per group. *p<0.05; **p<0.01; ***p<0.001 (One-way ANOVA with multiple comparisons, Fisher's LSD test).

FIG. 9. Immunohistochemical analysis of SARS-CoV-2 nucleoprotein (NP) antigen in lung tissues. A mild to strong positive immunoreactions were observed in lungs at 3 dpi from HPIV3-wt vector control, HPIV3/full S, HPIV3/S1, HPIV3/RBD1 and HPIV3/RBD2 groups. Mostly a mild immunoreaction was observed in lungs at 14 dpi from HPIV3-wt vector control, HPIV3/full S, HPIV3/S1, HPIV3/RBD1 and HPIV3/RBD2 groups. Magnification: 4×.

FIG. 10. Cytokine profile in lungs of vaccinated hamsters following SARS-CoV-2 challenge. The levels of cytokines (pg/ml) were determined in lung tissue homogenates by ELISA. A. Day 3 post-infection. B. Day 14 post-infection. Data represent mean±SEM of 4 animals per group. *p<0.05; **p<0.01; ***p<0.001; ****p<0.0001 (One-way ANOVA with multiple comparisons, Fisher's LSD test).

FIG. 11. Cytokine profile in lungs of vaccinated hamsters following SARS-CoV-2 challenge. The levels of cytokines (pg/ml) were determined in lung tissue homogenates by ELISA. A. Day 3 post-infection. B. Day 14 post-infection. Data represent mean±SEM of 4 animals per group. *p<0.05; **p<0.01; ***p<0.001; ****p<0.0001 (One-way ANOVA with multiple comparisons, Fisher's LSD test).

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Alternative vaccination strategies against SARS-CoV-2 are urgently needed to stop virus dissemination in human population and curb an ongoing pandemic of coronavirus-induced disease (COVID-19). While the currently approved SARS-CoV-2 vaccines are administered by intramuscular injection, a mucosal immunization may be necessary to restrain virus at the portal of entry and prevent an establishment of the lower respiratory tract infection. Described herein is a human parainfluenza virus (HPIV) vector platform to generate constructs expressing the full spike(S) protein of SARS-CoV-2, its S1 subunit, or the receptor-binding domain. The constructs were tested in golden Syrian hamsters as single-dose intranasal vaccines. Certain constructs induced high titers of neutralizing antibodies, and robust tissue-resident Th1 and CD8 T cell responses in lungs and spleen.

A full-length S protein construct induced potent neutralizing activity not only against the parental virus used in vaccine design (WA1/2020), but also against SARS-CoV-2 variants of concern (VOC). Using the peptide microarray, a comprehensive analysis of linear epitopes for vaccine-elicited antibodies specific to the S protein was performed. Conserved epitopes were identified at the junction of S2′ cleavage site and fusion peptide, and next to the heptad repeat 1. These epitopes likely contributed the serum neutralizing activity observed against SARS-CoV-2 variants by interfering with SARS-CoV-2 fusion machinery.

Induction of effective tissue-resident CD8. T cell response in the lungs by the vaccine has two advantages. First, CD8 T cells provide additional protection against infection at the portal of entry. Second, mutations in the S protein of SARS-CoV-2 variants which emerge as a result of selective pressure of antibodies are unlikely to significantly affect CD8. T cell epitopes. As such, lung-resident cytotoxic T cell response is likely to contribute equally well to the protection against both WA1/2020 and VOC.

Following SARS-CoV-2 challenge, hamsters vaccinated with the full S construct were completely protected from the disease and any detectable viral replication in the lungs. Immunized animals demonstrated gene expression pattern closely resembling the naïve group with a striking downregulation of the inflammatory pathways. A comprehensive analysis of secreted cytokine levels in the lungs and global transcriptome response demonstrated that HPIV3-based vaccination is a promising approach to minimize the excessive inflammatory response while preserving the protective immunity required for the effective clearance of the viral burden in the lungs after challenge infection. These data emphasize the advantages of respiratory tract vaccination against COVID-19 and inform the design of mucosal SARS-CoV-2 vaccines.

A panel of HPIV3-based constructs expressing the SARS-CoV-2 full-length spike glycoprotein or its fragments was developed and evaluated as single-dose IN vaccines in the golden Syrian hamster model of COVID-19. The data demonstrated strong SARS-CoV-2 neutralizing antibody and T cell responses induced by the full S construct. Although RBD is the immunodominant region eliciting SARS-CoV-2-neutralizing antibodies (Jiang et al., 2020; Premkumar et al., 2020), and that the vast majority (˜90%) of neutralizing antibodies detected after a natural human infection bind to RBD (Piccoli et al., 2020), only the full S vaccine, but not other constructs expressing the S1 protein subunit or any of the two versions of RBD, was able to induce strong immune responses that protected animals against the infection. However, membrane-anchored form of the S1 subunit expressed by a replication-competent vesicular stomatitis virus (VSV) (Malherbe et al., 2021) or inactivated rabies virus (RABV)-vectored vaccine (Kurup et al., 2021) induced virus-neutralizing antibody response and protected animals from SARS-CoV-2 challenge. Furthermore, the transmembrane RBD presented as a chimeric minispike in VSV replicon (Hennrich et al., 2021), virus spike RBD fused together as a tandem repeat (Dai et al., 2020), or RBD trimerized via fusion to the trimerization domain of T4 (Routhu et al., 2021) were also shown to elicit neutralizing antibodies in BALB/c mice. Thus, the membrane anchored or oligomerized forms of RBD displayed by these vaccines could have better preserved the conformation of B cell epitopes and thereby immunogenicity against S1 or RBD.

Despite good efficacy of multiple IM vaccines against SARS-CoV-2, a direct mucosal delivery may be necessary to attain robust protective immunity in the lungs. In BALB/c mice, only an IN inoculation, but not an IM injection, of a replication-incompetent recombinant adenovirus serotype 5 that carries the full-length SARS-CoV-2 S protein (Ad5-S-nb2) was able to elicit neutralizing antibody response in the bronchoalveolar lavage fluid (Feng et al., 2020). In the study with an integrase-deficient, non-integrative version of lentivirus expressing the full-length S protein administered as IM prime/IN boost vaccine, the boost immunization significantly improved protection against virus replication in lungs of the SARS-CoV-2-infected hamsters, even though it did not improve the serum neutralizing activity. Nevertheless, the complete protection was still not achieved (Ku et al., 2021). In another study, an IN vaccination with a chimpanzee adenovirus (simian Ad-36) encoding the S protein induced higher serum neutralizing titers, better protected hamsters, and was more potent in reduction of viral load when compared to IM vaccination (Bricker et al., 2021). However, even an IN vaccination with the construct was unable to completely prevent virus replication in the lungs of SARS-CoV-2-infected hamsters (Bricker et al., 2021) or rhesus macaques (Hassan et al., 2021a). In certain embodiments of the current invention, a single dose of HPIV3/full S provided excellent protection of the lower respiratory tract against virus replication, as evidenced by the lack of any detectable infectious virus or viral RNA at 3 dpi. Furthermore, very limited pathologic changes and mild antigen staining were observed in the lungs of hamsters vaccinated with the full S construct. Given the extremely high susceptibility of hamsters to SARS-CoV-2, the abrogation of virus replication in hamsters vaccinated with the full S construct is presumably attributed to effective neutralization of the virus by vaccine-induced antibodies at the mucosal surfaces. Consistently, HPIV3/full S vaccine reduced replication of virus in nasal turbinates to barely detectable levels. Even with IN route of vaccine delivery, it is difficult to achieve complete protection against SARS-CoV-2 in the upper respiratory tract (Bricker et al., 2021; Feng et al., 2020; Hassan et al., 2021a; Hassan et al., 2020; Hassan et al., 2021b; Lu et al., 2021). When high concentrations of potent RBD-specific neutralizing antibodies were applied intranasally 12 hours prior to challenge, the robust SARS-CoV-2 infection was still seen at 4 dpi in nasal turbinates, but not in the lungs of hamsters (Zhou et al., 2021). However, in two studies using the ferret animal model, full protection of the upper respiratory tract was observed after immunization (simultaneous oral and IN delivery) with a replication-deficient adenovirus serotype 5 encoding the spike protein (Ad5-nCoV) (Wu et al., 2020), or IN immunization with parainfluenza virus 5 expressing the S protein (An et al., 2021), although in the latter study virus transmission from vaccinated to naïve animals still occurred. In the first reported ferret challenge experiment (Kim et al., 2020), viral shedding was mainly observed in the upper respiratory tract, but infectious viral titers were low. The differences in disease severity among the animal models used to test SARS-CoV-2 vaccines, with the ferret model reproducing subclinical or mild human infections and the hamster model recapitulating lung pathology seen in patients with severe COVID-19 (Lee and Lowen, 2021), complicates the direct comparison of vaccine candidates.

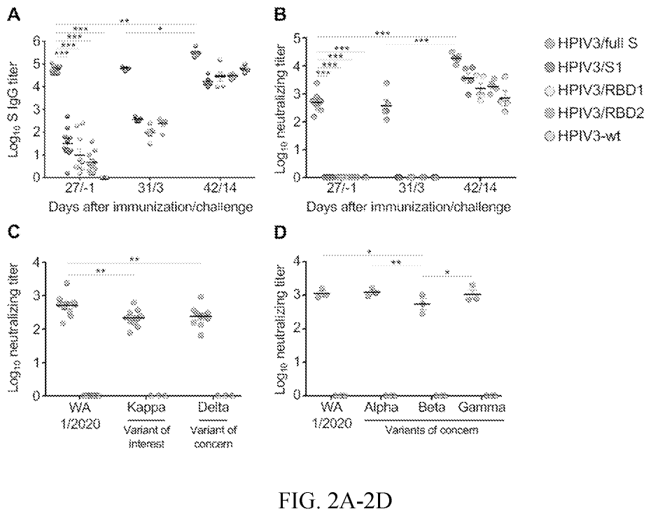

Using a peptide microarray, linear S protein epitopes were identified and targeted by vaccine-induced serum IgG. Five major linear epitopes have been identified, with 4 of them located outside RBD. The epitope found within RBD was outside of the receptor-binding motif, and antibodies to this epitope were detected in only one out of 10 pre-challenge serum samples from vaccine-immunized hamsters, with one of the lowest neutralizing titers across the group. Of note, early B cell response to the S protein analyzed from a COVID-19 patient was polyclonal and at epitopes mostly outside of the RBD (Seydoux et al., 2020). The linear epitope landscape of the SARS-CoV-2 spike protein constructed from 1,051 COVID-19 patients does not contain any relevant RBD epitopes (Li et al., 2021), suggesting that most of the epitopes in RBD are conformational. Epitope II in a close proximity to the S1/S2 cleavage site (669-683 aa) identified in our study overlaps with S1-111 (661-672 aa) and S1-113 (673-684 aa) epitopes from the epitope landscape (Li et al., 2021). Presumably, antibodies targeting this epitope can interfere with S1/S2 cleavage during the S protein processing, thus contributing the neutralizing serum activity. Interestingly, the antibody which blocks cleavage of Ebola virus (EBOV) GP into GP1 and GP2 subunits (Misasi et al., 2016) was shown protective in a non-human primate model against otherwise lethal EBOV infection (Corti et al., 2016). Epitope III which covers the S2′ cleavage site and fusion peptide (813-827 aa) overlaps with S2-22 (812-823 aa), S2-23 (818-829 aa) (Li et al., 2021) and S21P2 (809-826 aa) epitopes (Poh et al., 2020) identified when analyzing sera from COVID-19 patients. The antibodies against this epitope can neutralize SARS-CoV-2 (Poh et al., 2020), likely by blocking the cleavage and disturbing the function of FP. The cleavage at S2′ site is an important step to prime the S protein for membrane fusion. An inhibitor of TMPRSS2, serine protease which can mediate this cleavage, blocks SARS-CoV-2 infection of lung cells (Hoffmann et al., 2020). Most of the analyzed pre-challenge serum samples from hamsters vaccinated with HPIV3/full S construct (7 out of 10) contained antibodies with binding sites located next to HR1 (Table 1). The antibodies targeting HR1/2 may block the conformational changes that are essential for effective virus-cell fusion (Liu et al., 2004). Interestingly, antibodies that recognize parts of the viral envelope responsible for membrane fusion have been also identified for other viruses. Thus, 5 out of 6 most potent broadly neutralizing ebolavirus antibodies were shown to contain epitopes in the internal fusion loop of GP (King et al., 2019). Human monoclonal antibody BDBV259 binding to internal fusion loop of the Bundibugyo ebolavirus GP was shown to inhibit virus cell entry (Ilinykh et al., 2018). Overall, the epitopes found in S2 subunit are highly conserved across virus variants, and likely contribute neutralization of WA1/2020 and VOCs (FIG. 2B-D) by interfering with viral fusion machinery.

Although antibody response is important for neutralization of virus, the involvement of cell-mediated responses in SARS-CoV-2 clearance cannot be underestimated (McMahan et al., 2021). HPIV3-vectored vaccine induced both tissue-resident CD8⁺ T cell and Th1 immune responses in the lung tissues. The excessive Th2 cytokine-biased responses and inadequate Th1-biased T cell response were shown to contribute the immunopathology during SARS-COV infection in animal models (Bolles et al., 2011; Honda-Okubo et al., 2015; Tseng et al., 2012). Induction of effective tissue-resident CD8⁺ T-cell response in the lungs by the vaccine has two important advantages. First, CD8⁺ T-cells provide additional protection against infection at the portal of entry. Second, mutations in the S protein of SARS-CoV-2 variants emerge as a result of selective pressure of antibodies (Andreano and Rappuoli, 2021) rather than cytotoxic T cells, and therefore are unlikely to significantly affect sensitivity of CD8⁺ T cell epitopes to cell-mediated response. As such, the antiviral effect of lung-resident cytotoxic T cell response is likely to equally contribute to protection against both WA1/2020 and VOCs. Vaccination with HPIV3/full S elicited increase in both IFNγ and IL-12 following SARS-CoV-2 challenge. Both IFNγ and IL-12 activate macrophages and NK cells and increase antiviral activity of other tissue cells by upregulation of MHC class I/II molecules (Murphy and Weaver, 2017). On the other hand, vaccination limited the secretion of the proinflammatory cytokines TNFα and IL-6, which are involved in the acute inflammatory response, after the challenge. Supporting this, patients with severe COVID-19 had markedly higher levels of IL-6 and TNFα pro-inflammatory cytokines compared to patients with mild-to-moderate disease (Huang et al., 2020; Zhang et al., 2020). Furthermore, the bulk transcriptome analysis confirmed that HPIV3/full S vaccine prevented an overexpression of multiple chemokines and cytokines and subsequent influx of inflammatory cells including granulocytes and interstitial macrophages, which is a hallmark of COVID-19 pathogenesis (Huang et al., 2020; Zhang et al., 2020). Consistently, the histopathological analysis revealed a reduction in overall pneumonic changes including the extent of cellular infiltration and changes in vascular and airway compartments. Importantly, the presence of interstitial macrophages and granulocytes was lower in lungs of animals that were vaccinated with HPIV3/full S construct. The interstitial macrophages are attributed for excessive inflammatory response and tissue pathology (Byrne et al., 2015; Mosser and Edwards, 2008). Finally, no signs of increased pathology due to potential antibody-dependent enhancement were observed in vaccinated animals. Nevertheless, further investigation of this vaccine in additional animal models is required prior to considering clinical trials.

Essentially all humans are infected with HPIV3 during early childhood. Therefore, there is a concern that efficacy of HPIV3-vectored vaccines in adults might be reduced due to the prevalence of preexisting immunity against HPIV3 acquired by natural exposure (Bukreyev et al., 2006a). However, in guinea pigs that had previously been infected with HPIV3, immunization with a single dose of HPIV3 expressing EBOV GP (HPIV3/EboGP) induced titers of EBOV-specific serum antibodies that nearly equaled those detected in HPIV3-naïve animals (Yang et al., 2008). HPIV3/EboGP was also shown to replicate, although at a reduced level, in the respiratory tract of rhesus macaques, despite the preexisting immunity to vaccine vector, and elicit EBOV-specific neutralizing response. Remarkably, the neutralizing titers were even higher in HPIV3-immune monkeys after two vaccine doses each administered by the combined IN and intratracheal route, compared to HPIV3-naïve animals (Bukreyev et al., 2010). Further studies are required to determine the effects of HPIV3 preexisting immunity on protection elicited by HPIV3-vectored vaccines. However, in any case, HPIV3/full S is a good candidate for a pediatric needle-free bivalent vaccine against SARS-CoV-2 and HPIV3, as it is based on the JS strain of HPIV3 which is expected to be naturally attenuated in humans (Clements et al., 1991).

Given an unprecedented global spread of SARS-CoV-2 and emergence of the new virus variants resistant to neutralization, protection induced by tissue-resident CD8⁺ T cells in the lungs induced by the vaccine is unlikely to be significantly reduced by mutations present in the current and future SARS-CoV-2 variants. In addition, HPIV3 vector platforms may be considered for boost IN vaccination following IM prime with currently approved vaccines to stimulate balanced systemic and local immune response in the respiratory tract. The data obtained suggest HPIV3/full S is a promising vaccine candidate and justify its preclinical evaluation.

### I. Human Parainfluenza Virus Vectors/Constructs

Human parainfluenza virus type 3 (HPIV3) (family Paramyxoviridae) is an enveloped virus with a single-stranded negative-sense RNA genome. It is a common pediatric virus which infects the respiratory tract causing a mild respiratory disease and does not spread to other tissues. These features make HPIV3-based vaccines suitable for intranasal (IN) administration, but also ensure greater safety (Bukreyev et al., 2006a). Therefore, HPIV3 is well-suited as a vector for pathogens that use the respiratory tract as a portal of entry, such as SARS-CoV-2.

Described herein is a panel of HPIV3 constructs expressing the full S protein of SARS-CoV-2, its S1 subunit, or RBD expressed from an added transcriptional unit. The HPIV3 genome consists of six distinct transcriptional units, which encodes for one or more genes. Each transcriptional unit is separated by a gene end (5′-AAAUAAGAAAAA-3′ (SEQ ID NO:11)), intercistronic (5′-CUU-3′), and gene start sequences (5′-AGGAUUAAAG-3′ (SEQ ID NO:12)). All constructs were successfully recovered and shown to produce/express the expected proteins in infected cells. The efficiency of vaccine constructs was tested in a hamster animal model as a single IN dose. The full S-containing vaccine, HPIV3/full S, was able to induce strong neutralizing antibody titers and tissue-resident T cell responses resulting in barely detectable SARS-CoV-2 replication in the lungs and alleviated tissue pathologic changes and body weight loss upon challenge. Importantly, the tissue-resident cytotoxic CD8⁺ T cell response elicited by the vaccine in the lungs represents an additional barrier against the virus. Furthermore, this barrier is expected to be less sensitive to mutations present in SARS-CoV-2 variants than antibody responses. “Expression” refers to the transcription and stable accumulation of mRNA. Expression may also refer to the production of protein. In certain aspects, the HPIV3 vectors encode a heterologous segment encoding all or part of the SARS-CoV-2 spike protein. As used herein, “heterologous” refers to a nucleotide sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g. present in a different copy number, different location, and/or under the control of different regulatory sequences, than that found naturally in nature.

Vaccines described herein comprise HIPV polynucleotide (platform or construct) encoding one or more SARS-CoV-2 antigens. The sequences provided may be presented as DNA sequences, deoxyribose adenine, guanine, thymine, cytosine (AGTC) and/or RNA sequences ribose adenine, guanine, uracil, cytosine (AGUC); one of skill would readily identify the RNA or DNA counterpart.

Vaccine compositions of the invention may comprise other components including, but not limited to, adjuvants. Adjuvants may also be administered with or in combination with one or more vaccine. In one aspect, an adjuvant acts as a co-signal to prime T-cells and/or B-cells and/or NK cells as to the existence of an infection. Adjuvants may be co-administered by any route. In certain aspects adjuvants can be co-administered or co-formulated with the vaccine compositions described herein. Adjuvants useful in the present invention may include, but are not limited to, natural or synthetic adjuvants. Adjuvants can be selected from any of the classes (1) mineral salts, e.g., aluminium hydroxide and aluminium or calcium phosphate gels; (2) emulsions including: oil emulsions and surfactant based formulations, e.g., microfluidised detergent stabilized oil-in-water emulsion, purified saponin, oil-in-water emulsion, stabilized water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensoactive compounds; (9) carbohydrates; or combinations thereof.

An “effective amount” of the vaccine composition is provided based, at least in part, on the target tissue, target cell type, means of administration, physical characteristics of the vector (e.g., size, and extent of modified nucleosides) and other components of the vaccine, and other determinants. In general, an effective amount of the vaccine composition provides an induced or boosted immune response as a function of antigen production in the subject or the subject's cells.

Activation of the Immune Response. According to various embodiments, the vaccine comprising the polynucleotides disclosed herein may act as a vaccine. As used herein, a “vaccine” refers to a composition, for example, a substance or preparation that stimulates, induces, causes or improves immunity in an organism, e.g., a mammalian organism (a human, etc.). Preferably, a vaccine provides immunity against one or more diseases or disorders, including prophylactic and/or therapeutic immunity. Vaccines may be administered prophylactically or therapeutically as part of an active immunization scheme to healthy individuals or early in infection during the incubation phase or during active infection after onset of symptoms.

In one embodiment, the polynucleotides of the vaccine may be administrated with other prophylactic or therapeutic compounds. As a non-limiting example, the prophylactic or therapeutic compound may be an adjuvant or a booster. As used herein, when referring to a prophylactic composition, such as a vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In certain aspects, the polynucleotides of the invention may be administered intranasally.

In certain aspects, vaccines described herein can be used as memory booster vaccines and are administered to boost antigenic memory across a time period of 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more years.

### II. Vaccine Polynucleotides

According to certain embodiments, the polynucleotides encode at least one polypeptide or peptide of interest (an antigen or immunogen). Antigens of the present invention may be derived from SARS-CoV-2. In certain embodiments, the antigen is derived from the spike protein (S protein) of SARS-CoV-2, in particular the antigen is all or a portion of the S protein, including S1 subunit or receptor binding domain or fragments thereof.

Certain embodiments are directed to nucleic acid molecules, e.g., viral vaccine vector, that encode one or more peptides or polypeptides of interest. Such peptides or polypeptides serve as an antigen or antigenic molecule. The term “nucleic acid,” in its broadest sense, is a polymer of nucleotides. These polymers are often referred to as polynucleotides. Nucleic acids or polynucleotides of the invention include, but are not limited to, ribonucleic acids (RNAs), which may or may not include ribonucleotide analogs or modifications.

In one embodiment, the polynucleotide has a region or segment encoding at least one polypeptide of interest. As used herein, such a region may be referred to as a “coding region” or “region encoding” or “open reading frame (ORF)”.

As used herein, “polypeptide” means a polymer of amino acid residues (natural or unnatural) linked together most often by peptide bonds. The term, as used herein, refers to proteins, polypeptides, and peptides of any size, structure, or function. In one embodiment, the polypeptides of interest are antigens encoded by the polynucleotides as described herein.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to polypeptides are those with one or more amino acids in the native or starting amino acid sequence removed. Ordinarily, deletional variants will have one or more amino acids deleted in a particular region of the molecule.

Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402). Other tools are described herein, specifically in the definition of “Identity.”

Default parameters in the BLAST algorithm include, for example, an expect threshold of 10, Word size of 28, Match/Mismatch Scores 1, −2, Gap costs Linear. Any filter can be applied as well as a selection for species specific repeats, e.g., Homo sapiens.

Codon Optimization. The polynucleotides contained in the vaccines of the invention, their regions or parts or subregions may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide. Codon optimization tools, algorithms and services are known in the art, non-limiting examples include services from GeneArt (Life Technologies), DNA2.0 (Menlo Park Calif.) and/or proprietary methods. In one embodiment, the ORF sequence is optimized using optimization algorithms.

### III. Pharmaceutical Vaccine Compositions

The present invention provides pharmaceutical compositions including vaccines and vaccine compositions and/or complexes optionally in combination with one or more pharmaceutically acceptable excipients. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21′ ed., Lippincott Williams & Wilkins, 2005 (incorporated herein by reference in its entirety).

In some embodiments, compositions are administered to humans, human patients or subjects. For the purposes of the present disclosure, the phrase “active ingredient” generally refers to the vaccines or the polynucleotides contained therein, e.g., antigen-encoding polynucleotides, for example, RNA polynucleotides, to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 98%. e.g., between 0.5 and 50%, between 1-30%, between 5-80%, at least 80% (w/w) active ingredient.

Formulations. The vaccines of the invention can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with vaccines (e.g., for transplantation into a subject), hyaluronidase, nanoparticle mimics and combinations thereof.

Accordingly, the formulations of the invention can include one or more excipients, each in an amount that increases the stability of the vaccine, increases cell transfection by the vaccine, increases the expression of polynucleotides encoded protein, and/or alters the release profile of polynucleotide encoded proteins. Further, the polynucleotides of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

Excipients. NAV pharmaceutical formulations may additionally comprise a pharmaceutically acceptable excipient, which, as used herein, includes, but are not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, antioxidants, osmolality adjusting agents. pH adjusting agents and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, Md., 2006; incorporated herein by reference in its entirety). The use of a conventional excipient medium may be contemplated within the scope of the present disclosure, except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Cryoprotectants. In some embodiments, NAV formulations may comprise cryoprotectants. As used herein, there term “cryoprotectant” refers to one or more agent that when combined with a given substance, helps to reduce or eliminate damage to that substance that occurs upon freezing. In some embodiments, cryoprotectants are combined with NAVs in order to stabilize them during freezing. Frozen storage of NAVs between −20° C. and −80° C. may be advantageous for long term (e.g. 36 months) stability of polynucleotide. In some embodiments, cryoprotectants are included in NAV formulations to stabilize polynucleotide through freeze/thaw cycles and under frozen storage conditions. Cryoprotectants of the present invention may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

Bulking Agents. In some embodiments, NAV formulations may comprise bulking agents. As used herein, there term “bulking agent” refers to one or more agents included in formulations to impart a desired consistency to the formulation and/or stabilization of formulation components. In some embodiments, bulking agents are included in lyophilized NAV formulations to yield a “pharmaceutically elegant” cake, stabilizing the lyophilized NAVs during long term (e.g. 36 month) storage. Bulking agents of the present invention may include, but are not limited to sucrose, trehalose, mannitol, glycine, lactose and/or raffinose. In some embodiments, combinations of cryoprotectants and bulking agents (for example, sucrose/glycine or trehalose/mannitol) may be included to both stabilize NAVs during freezing and provide a bulking agent for lyophilization.

Dosing. The present invention provides methods comprising administering vaccines and in accordance with the invention to a subject in need thereof. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 10³ to 10⁸ pfu per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens may be used.

According to the present invention, vaccines may be administered in split-dose regimens. As used herein, a “split dose” is the division of single unit dose or total daily dose into two or more doses, e.g., two or more administrations of the single unit dose. As used herein, a “single unit dose” is a dose of any therapeutic administer in one dose/at one time/single route/single point of contact, i.e., single administration event. As used herein, a “total daily dose” is an amount given or prescribed in 24 hr period. It may be administered as a single unit dose. In one embodiment, the NAVs of the present invention are administered to a subject in split doses. The NAVs may be formulated in buffer only or in a formulation described herein.

### IV. Kits and Devices

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

In one aspect, the present invention provides kits comprising the vaccine(s) of the invention.

The kit may further comprise packaging and instructions and/or a delivery agent to form a formulation composition. The delivery agent may comprise a saline, a buffered solution, or a delivery agent.

In one embodiment, the buffer solution may include sodium chloride, calcium chloride, phosphate and/or EDTA. In another embodiment, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose. In a further embodiment, the buffer solutions may be precipitated or it may be lyophilized. The amount of each component may be varied to enable consistent, reproducible higher concentration saline or simple buffer formulations. The components may also be varied in order to increase the stability of polynucleotides in the buffer solution over a period of time and/or under a variety of conditions.

Devices. The present invention provides for devices which may incorporate vaccines comprising polynucleotides that encode polypeptides of interest, e.g., encode antigenic polypeptides.

Devices for administration may be employed to deliver the vaccine of the present invention according to single, multi- or split-dosing regimens taught herein.

### V. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A single intranasal dose of human parainfluenza virus type 3-vectored vaccine induces effective antibody and tissue-resident T cell response in the lungs and protects hamsters against SARS-CoV-2

### A. Results

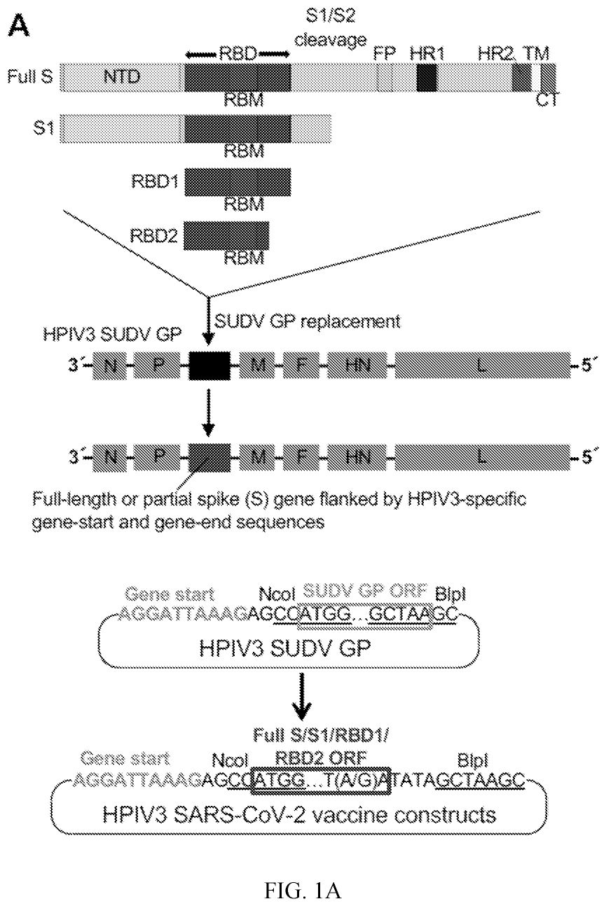

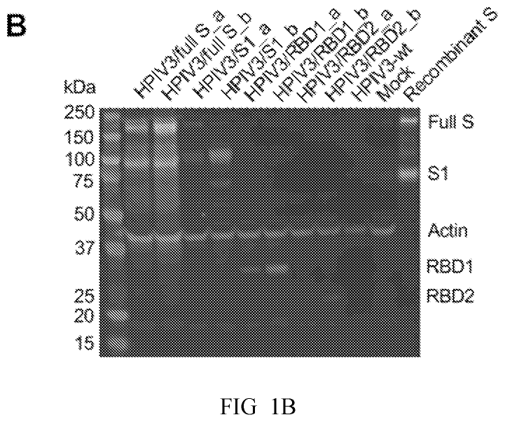

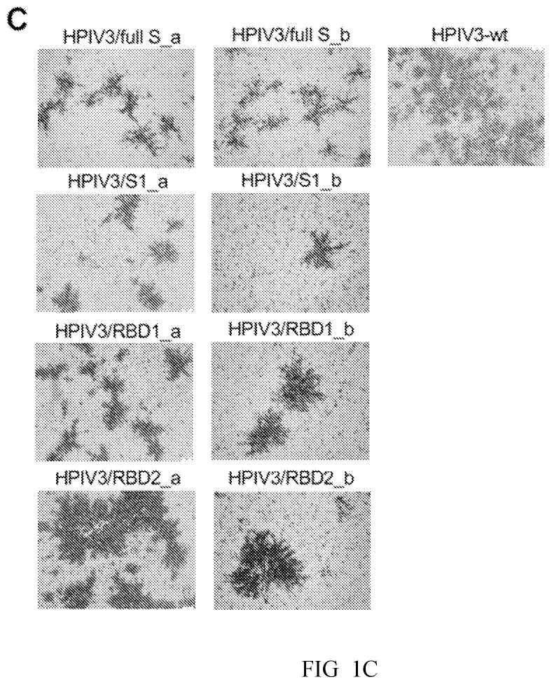

Introduction of SARS-CoV-2 S gene or its fragments into HPIV3 full-length clone results in recovery of replication-competent vaccine constructs. To generate candidate vectored vaccines, HPIV3-based platform was used. A SARS-CoV-2 S transcriptional cassette was generated by adding HPIV3-specific gene-start and gene-end transcriptional signals upstream and downstream the open reading frame and incorporated it in the P-M intergenic sequence (FIG. 1A). Constructs were also generated expressing subunit S1 and receptor binding domain (RBD). Since the amino acid positions determining RBD location do vary across published data, constructs were generated expressing the “long” (RBD1, 319-591 aa) and “short” (RBD2, 319-529 aa) versions of RBD (Shang et al., 2020; Wrapp et al., 2020). Two versions of the S gene cDNA were used: codon-optimized and not optimized for eucaryotic translation, resulting in set “a” and set “b” constructs, respectively. Totally, eight constructs were obtained (see Materials and methods): HPIV3/full S_a, HPIV3/full S_b, HPIV3/S1_a, HPIV3/S1_b, HPIV3/RBD1_a, HPIV3/RBD1_b, HPIV3/RBD2_a and HPIV3/RBD2_b. All of them were successfully recovered upon transfection of BSR-T7 cells. Expression of the expected S proteins in virus-infected cells was confirmed by western blot (FIG. 1B). In LLC-MK2 cells infected with HPIV3/full S constructs (the first 2 lanes), both full-length S and its cleaved product, S1, were detected, indicating the expected cleavage of the S protein. All constructs were shown to form viral plaques immunostained with HPIV3-specific antibodies in LLC-MK2 cell monolayers (FIG. 1C). As expected, the plaque size increased from full S to S1, RBD1, and RBD2 according to the reduction of length of the inserted S gene fragment.

The full S vaccine induces a robust antibody response. Since the set “b” constructs showed the uniformly higher expression of S gene fragments than the set “a” counterparts (FIG. 1B), the “b” set of constructs was further selected for immunogenicity and protection studies in the golden Syrian hamster model of SARS-CoV-2 infection (referred to without “b” index thereafter) (Imai et al., 2020). Groups of 6-7-week-old hamsters (n=10 per group) were inoculated by the IN route with single doses of the constructs expressing full S, S1, RBD1, RBD2, or wild-type (wt) HPIV3 (empty vector as mock-vaccine control) at 10⁶ plaque-forming units (PFU) per animal in a total volume of 100 μl (approximately 50 μl into each nostril). The full S vaccine induced higher S-specific binding antibody titers than all other vaccines (FIG. 2A). The serum neutralization assay performed with mNeonGreen SARS-CoV-2 (strain WA1/2020) (Xie et al., 2020) revealed that only HPIV3/full S construct was able to induce neutralizing antibodies (FIG. 2B). These data show that a single IN dose of HPIV3-vectored full S vaccine induces a robust serum antibody response.

The full S vaccine induces a robust antibody response against SARS-CoV-2 variants of concern. To evaluate the breadth of neutralization of vaccine-induced antibodies, pre-challenge samples from HPIV3/full S group were tested against Kappa and Delta SARS-CoV-2 variants (FIG. 2C). Kappa variant is classified as the variant of interest by the US CDC, whereas Delta variant is classified as the variant of concern (VOC) due to its high infectivity (Konings et al., 2021). Samples from three animals in the HPIV3-wt group were picked randomly to serve as negative controls. Both Kappa and Delta variants were neutralized by the immune sera, although at a slightly lower extent compared to an original WA1/2020 strain. Finally, three samples with the highest WA1/2020-neutralizing titers were selected and tested against the biological SARS-CoV-2 isolates in a standard plaque reduction neutralization assay: WA1/2020, and three VOCs, Alpha, Beta and Gamma (FIG. 2D). The level of Alpha and Gamma variants' neutralization was comparable to that of WA1/2020, whereas Beta variant appeared to be less sensitive to serum antibodies. To summarize, it is shown that a single IN dose of HPIV3-vectored full S vaccine induces a robust and broad humoral immune response, which is capable of neutralizing not only the homologous virus strain, but also SARS-CoV-2 VOCs.

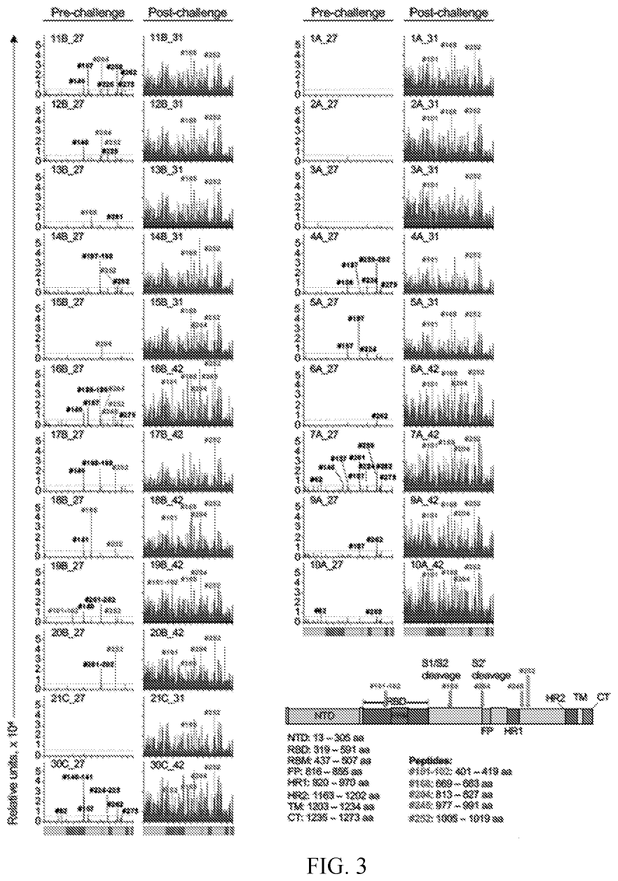

The vaccine-induced antibody response targets multiple linear epitopes across the S protein. The S protein linear epitopes targeted by hamster immune sera were elucidated using peptide microarrays of immobilized 15-mer oligopeptides covering the entire SARS-CoV-2 S sequence with 4 amino acid overlaps (FIG. 3, Table 1). Pre- and post-challenge samples from all animals in the HPIV3/full S and HPIV3-wt groups were analyzed. In addition, serum samples from two animals in HPIV3/S1 group with relatively high S IgG titers were also included (FIG. 2A). The slides with immobilized 15-mer oligopeptides overlapping the entire SARS-CoV-2 S sequence with 4 amino acid overlaps were incubated with serum samples, followed by incubation with secondary antibody conjugated with Cy5 fluorophore, and the mean fluorescent intensity (MFI) values for each individual peptide were recorded. The following S protein regions were identified as potential epitopes for vaccine-raised systemic IgG antibodies: 405-415 aa (the overlapping part of peptides #101 and #102; RBD, outside of the receptor-binding motif), 669-683 aa (peptide #168; in a close proximity to the S1/S2 cleavage site), 813-827 aa (peptide #204; overlapping S2′ cleavage site and N-terminus of the fusion peptide), 977-991 and 1005-1019 aa (peptides #245 and #252; adjacent to HR1). Antibodies to these S protein regions were further boosted by SARS-CoV-2 infection. Noteworthy, two samples with the highest pre-challenge neutralizing titers against the WA1/2020 strain demonstrated the presence of antibodies binding S2′ cleavage site and the fusion peptide. Seven out of 10 serum samples from HPIV3/full S group contained antibodies with binding sites located next to HR1 (Table 1). Another potential epitope was identified in vicinity to the S1/S2 cleavage site. None of the epitopes contain any N-linked glycosylation sites (Watanabe et al., 2020) or mutations identified in viral stocks of Gamma and Delta variants used for in vitro experiments. These data suggest that HPIV3-based SARS-CoV-2 vaccine induces antibodies spanning multiple linear epitopes in the S protein.

TABLE 1
Characteristics of individual pre-challenge serum samples from
HPIV3/full S_II group hamsters. The samples are ordered according
to their neutralizing titers, from highest to lowest.

| | Log₁₀ | Log₁₀ S | | | | | | |
|---|---|---|---|---|---|---|---|---|
| Animal | Neutralizing | IgG | Peptides (MFI, relative units) | | | | | |
| ID | titer* | titer** | #101 | #102 | #168 | #204 | #245 | #252 |
| 11B | 3.383 | 5.046 | *** | *** | *** | 7448 | *** | *** |
| 15B | 2.897 | 4.700 | *** | *** | *** | 11808 | *** | *** |
| 18B | 2.794 | 4.600 | *** | *** | 43856 | *** | *** | 10276 |
| 17B | 2.769 | 4.600 | *** | *** | *** | *** | *** | 20213 |
| 20B | 2.741 | 4.900 | *** | *** | *** | *** | *** | 36723 |
| 16B | 2.731 | 4.900 | *** | *** | *** | 7140 | 9705 | 17394 |
| 13B | 2.650 | 4.900 | *** | *** | 11135 | *** | *** | *** |
| 19B | 2.506 | 4.800 | 6564 | 8570 | *** | *** | *** | 7445 |
| 12B | 2.401 | 4.600 | *** | *** | *** | 7498 | *** | 14607 |
| 14B | 2.173 | 4.600 | *** | *** | *** | *** | *** | 6467 |

*FIG. 2B data;
**FIG. 2A data;
*** Below the background level

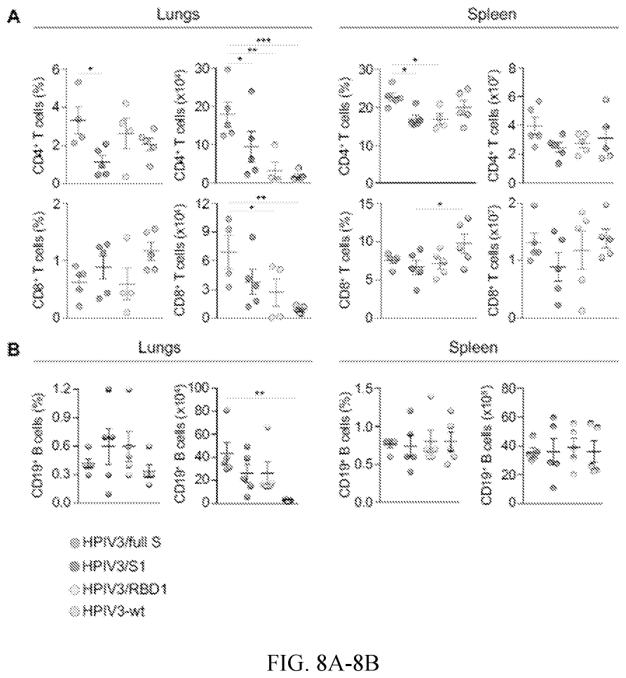

The full S vaccine induces robust Th1 and CD8′ T cell responses in lungs and spleen. Separate groups of hamsters were intranasally inoculated with HPIV3/full S, HPIV3/S1 or HPIV3/RBD1 constructs, or HPIV3-wt. After 28 days, vaccinated animals were euthanized, lungs and spleen were collected, and immune cells were isolated. Multi-parameter flow cytometry analysis demonstrated that the frequencies of CD4″ and CD8⁺ T cells in the lungs remained unchanged in vaccinated hamsters when compared to the control hamsters vaccinated with empty vector (FIG. 8A). However, the absolute numbers of these cells were higher in hamsters vaccinated with HPIV3/full S construct compared to other groups. In contrast to the lungs, no significant differences between HPIV3/full S and control group were observed in percentages and total T cell numbers in spleen. Similarly, the B cell numbers were higher in lungs of vaccinated animals compared to the vector-only control (FIG. 8B). However, B cell numbers in spleen did not differ between groups.

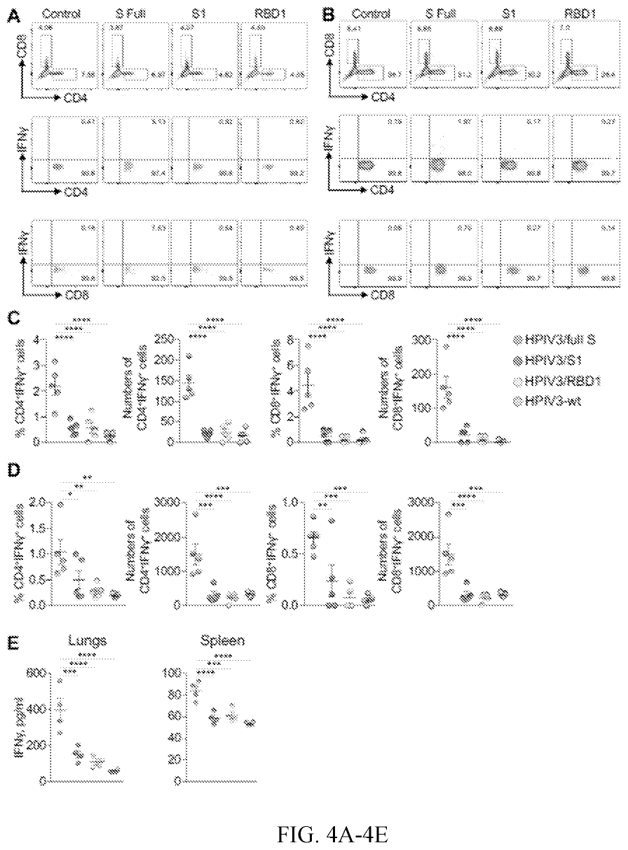

The immune cells isolated from organs were stimulated with a pool of SARS-CoV-2 S peptides, and IFNγ⁺ T-cells were quantified by flow cytometry (FIG. 4A-D). Significantly higher percentages and the total numbers of IFNγ⁺CD4⁺ T cells and IFNγ⁺CD8⁺ T cells in the lungs and spleen were detected in hamsters vaccinated with HPIV3/full S compared to all other groups. The levels of IFNγ secreted in the culture supernatants following peptide treatment were quantified by ELISA. Significantly greater levels of IFNγ were detected in supernatants of the lung cells in HPIV3/full S group compared to all other groups (FIG. 4E, left panel). Although the magnitude of IFNγ levels secreted by spleen cells was lower than that of lung cells, HPIV3/full S vaccine induced secretion of greater levels of IFNγ compared to the other constructs (FIG. 4E, right panel). These results demonstrate that HPIV3/full S induces robust tissue-resident memory Th1 and CD8⁺ T cell responses in lungs and spleen.

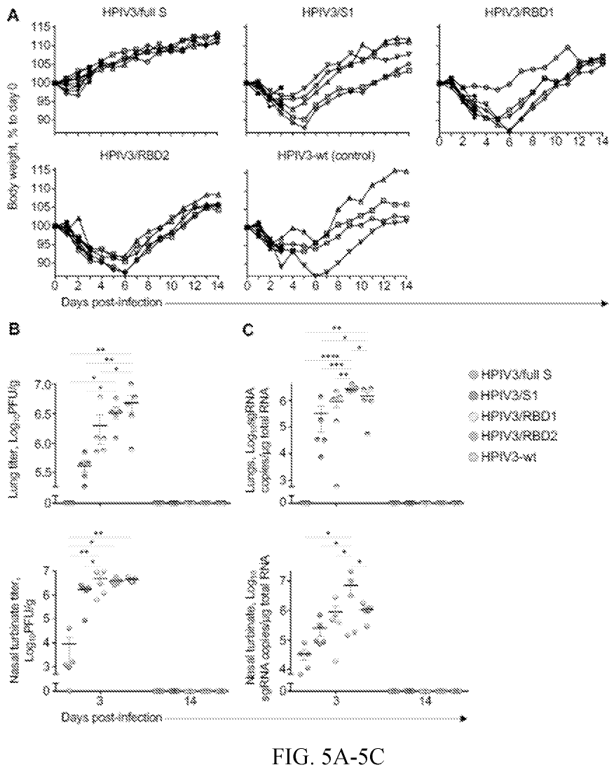

The HPIV3 full S vaccine construct protects hamsters from SARS-CoV-2 infection and disease. Four weeks after vaccination, hamsters were challenged by the IN route with 10⁵ PFU of SARS-CoV-2. Consistent with the immune response data, HPIV3/full S completely prevented the reduction of body weight upon infection. Animals from the other groups demonstrated weight loss reaching a maximum of approximately 10% on days 5-6 post-infection (dpi) (FIG. 5A). The hamsters were serially euthanized at 3 and 14 dpi to determine viral load (FIG. 5B) and viral RNA level (FIG. 5C) in tissues. Strikingly, no virus was detected in the lungs of HPIV3/full S immunized animals at 3 dpi by either plaque assay or qRT-PCR method. The HPIV3/S1 and HPIV3/RBD1 constructs also reduced the virus load in the lungs compared to the HPIV3-wt control at 3 dpi. Only HPIV3/full S construct significantly reduced viral titers in nasal turbinates when compared to other groups. At 14 dpi, SARS-CoV-2 was not detected in any animal, suggesting the resolution of active viral replication by this time point.

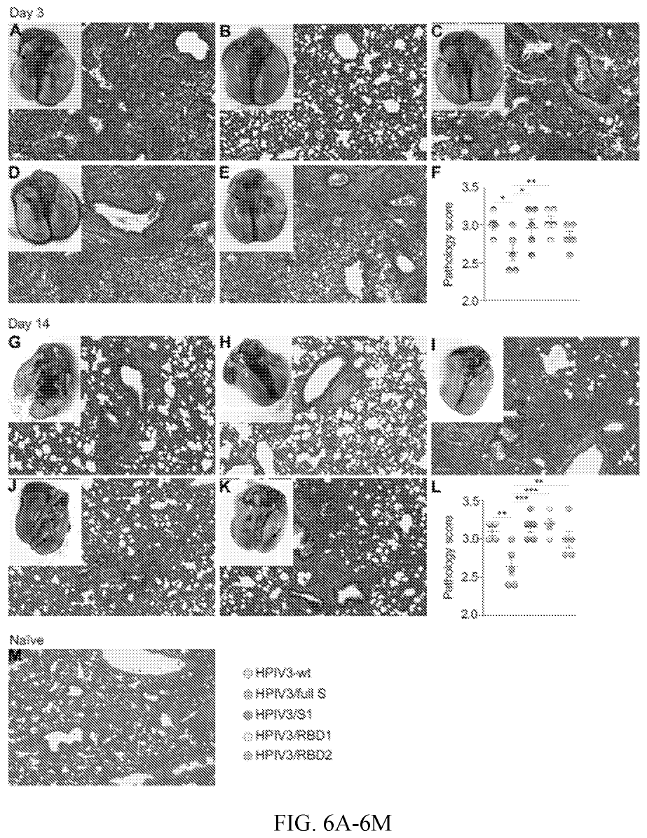

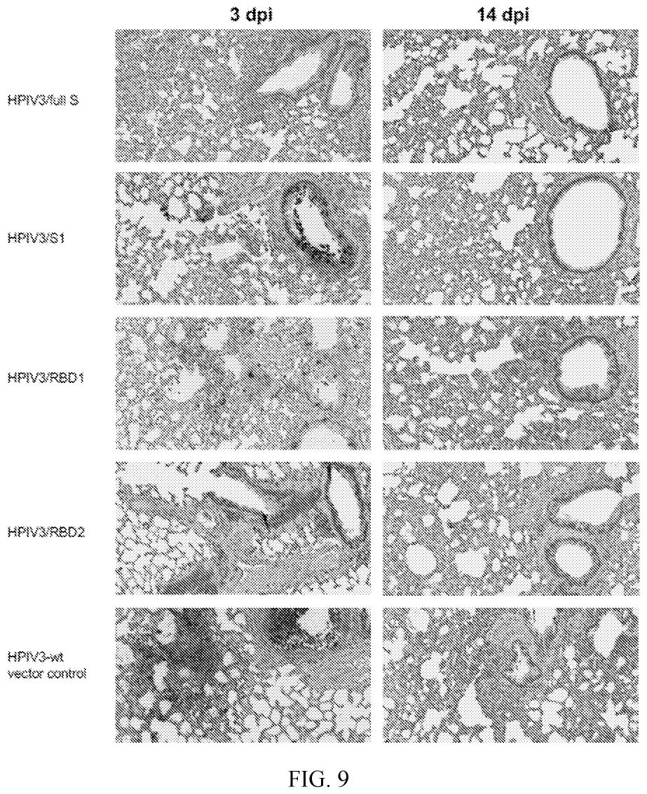

Immunohistochemical staining for viral antigen (NP) in lungs revealed mild to severe immunostaining pattern among different groups (FIG. 9; Table 2). At 3 dpi, a moderate to severe immunostaining was observed in all groups except HPIV3/full S which showed only barely detectable staining at a few places (FIG. 9, left), suggesting low-level transient replication of the virus. At 14 dpi, no-to-mild level of immunostaining was observed in all groups except for animals vaccinated with HPIV3/RBD1 and HPIV3/RBD2 which had mild-to-moderate staining (FIG. 9, right). These data are consistent with the viral load determined by plaque assay and qRT-PCR (FIG. 5B,C). On gross examination of the lungs, a generalized congestion and consolidatory foci were seen in the empty vector-vaccinated control animals euthanized at 3 dpi (FIG. 6A) or 14 dpi (FIG. 6G). These gross changes were absent or minimal in hamsters vaccinated with the HPIV3/full S construct (FIG. 6B,H), but the other groups had gross lesions comparable to that in the control animals (FIG. 6C-E, I-K). At 3 dpi, typical interstitial pneumonia was noticed in lungs from the vector-vaccinated control animals which was characterized by a severe inflammation with mononuclear cells and heterophils in the alveolar space, interstitial septa and airways septal thickening, perivascular cuffing and vascular endothelial cell damage (FIG. 6A). In contrast, in animals vaccinated with the HPIV3/full S construct, moderate inflammation with lesser vascular and airway changes and lesser numbers of inflammatory macrophages and heterophils were observed (FIG. 6B,F). Furthermore, in animals vaccinated with the HPIV3/S1 (FIG. 6C), HPIV3/RBD1 (FIG. 6D) or HPIV3/RBD2 (FIG. 6E) constructs, a moderate to severe interstitial pneumonic changes that are comparable to vector-vaccinated control animals were detected. On day 14, an interstitial pneumonia with marked septal thickening was prominent in vector-vaccinated control (FIG. 6G), as well as in HPIV3/S1 (FIG. 6I), HPIV3/RBD1 (FIG. 6J) and HPIV3/RBD2 groups (FIG. 6K). However, the inflammatory changes were mild-to-moderate in HPIV3/full S group (FIG. 6H,L), but a moderate septal thickening was still present in this group. In summary, vaccination with HPIV3/full S limits severe lung pathology and reduces the inflammatory changes. Thus, IN administration of a single dose of HPIV3/full S elicited protection in the upper respiratory tract and the lungs.

TABLE 2
Intensity of pattern for viral antigen.

| Vaccine construct | 3 dpi | 14 dpi |
|---|---|---|
| HPIV3-wt | ++/+++ | −/+ |
| HPIV3/full S_II | +/++ | −/+ |
| HPIV3/S1_II | ++/+++ | −/+ |
| HPIV3/RBD1_II | ++/+++ | +/++ |
| HPIV3/RBD2_II | +++ | +/++ |

Notes:
− none,
+ mild (<10% of the positive staining area under 4x field),
++ moderate (10-30% of the positive staining area under 4x field),
+++ severe (>30% of the positive staining area under 4x field).

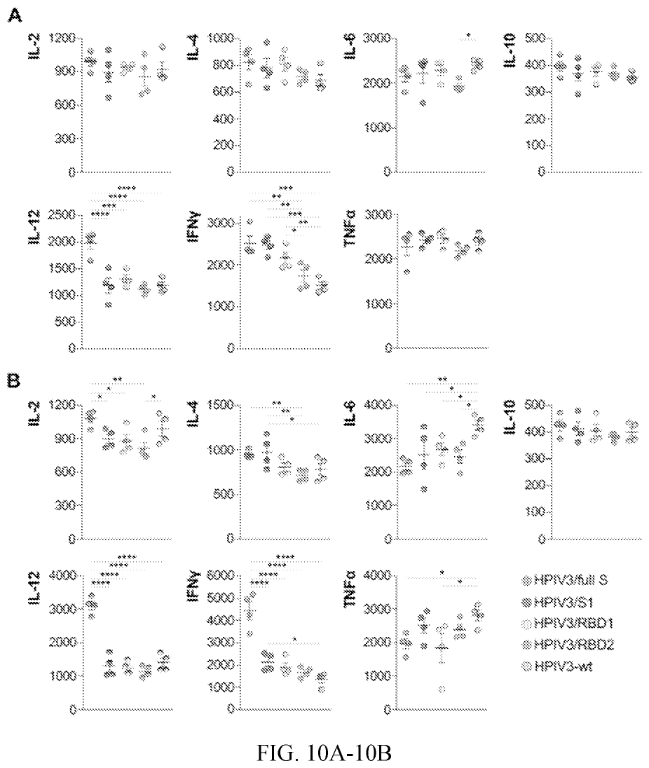

SARS-CoV-2 challenge of animals vaccinated with the full S vaccine construct induces Th1 environment in the lungs. The effects of the vaccination on cytokine profile in the lungs was tested post-challenge. The cytokine levels in lung homogenates were analyzed by ELISA. At 3 dpi, the levels of IL-2, IL-4, IL-6, IL-10 and TNFα were similar in all groups, except from the slight IL-6 reduction by HPIV3/RBD2 compared to the control group (FIG. 10A). However, the level of IL-12 was significantly higher in the HPIV3/full S group when compared to all other groups. Although the level of IFNγ was higher in HPIV3/full S than in HPIV3/RBD2 or vector-control group, it was not significantly different from HPIV3/S1 or HPIV3/RBD1 group. Interestingly, on day 14 post-infection, the levels of IL-12 and IFNγ were significantly higher in HPIV3/full S group compared to all other groups (FIG. 10B). At this time, there were no differences in IL-2 or IL-4 levels between HPIV3/full S and the control group, and IL-10 secretion remained similar in all groups. The IL-6 levels were reduced in all vaccinated animals compared to control animals. The decrease in TNFα secretion levels was observed in HPIV3/full S and HPIV3/RBD1 groups. The data demonstrate that SARS-CoV-2 challenge of HPIV3/full S vaccinated animals triggers Th1-type environment in the lungs, which is associated with protection.

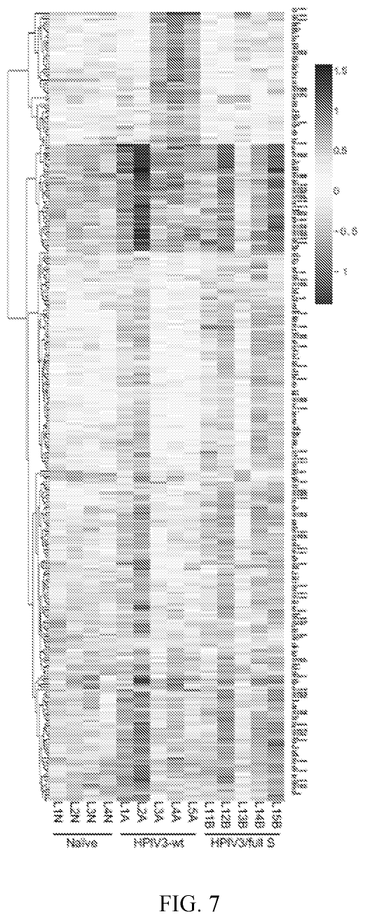

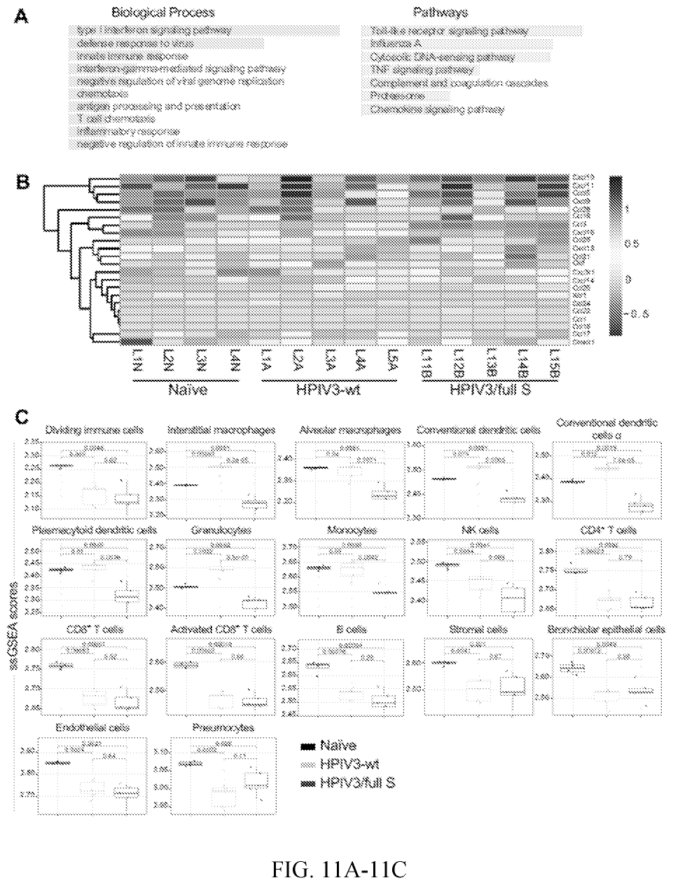

Vaccination with HPIV3 vaccine expressing the full S protein prevents inflammatory response in the lungs upon challenge. To characterize the global transcriptome response in lungs of vaccinated hamsters after the challenge, RNAseq was performed at 3 dpi comparing the HPIV3/full S group (n=5) to the HPIV3-wt control group (n=5). Lung samples from naïve hamsters (mock-infected with media, n=4) served as a baseline control for analysis. Heat map of the top 300 differentially expressed genes revealed a significant upregulation of 250 genes in HPIV3-wt group relative to the naive group, which included several chemokines and cytokines (FIG. 7), the data consistent with induction of inflammation and cytokine storm in COVID-19 patients (Huang et al., 2020; Zhang et al., 2020). In contrast, HPIV3/full S-vaccinated animals demonstrated a gene expression pattern closely resembling that in the naïve group, with a striking downregulation of multiple genes which contribute to the inflammation. Gene ontology analysis of the genes upregulated in HPIV3-wt group revealed a significant enrichment of several biological processes characteristic of an acute viral infection (FIG. 11A, left panel), including innate immune response, chemotaxis and inflammatory response. Similarly, pathway analysis revealed a significant enrichment of TLR, TNF and chemokine signaling pathways (FIG. 11A, right panel). Chemokine gene expression heat map showed a marked reduction of chemokine expression in vaccinated hamsters (FIG. 11B) when compared to upregulation in the HPIV3-wt group (Cxcl9, Cxcl10, Cxcl1 and Cxcl16).

To gain biological insights of the gene expression profiling data, deconvolution of the bulk RNAseq data was performed using pathway-level information extractor (PLIER) (Mao et al., 2019) to identify differences in cell-type proportion between the three groups. A significant increase in infiltration of myeloid cell populations: interstitial macrophages, activated DC (DCα) and granulocytes were detected in the mock-vaccinated infected animals compared to the naïve animals, suggesting a preponderance of acute inflammation. Vaccination with HPIV3/full S completely or partially prevented the infiltration of these cells, as well as alveolar macrophages, conventional DC (CDC), plasmacytoid DC (pDC) and monocytes (FIG. 11C). A significant reduction of lymphoid cell populations: natural killer (NK) cells, CD4⁺ T cells, CD8⁺ T cells, activated CD8⁺ T cells and B cells, as well as in stromal cells, bronchiolar epithelial cells and pneumocytes was also detected in mock-vaccinated animals, but vaccination did not alleviate these effects. Additionally, gene set enrichment analysis of the entire dataset was performed using GAGE pathway analysis (Luo et al., 2009) to identify enriched pathway categories, as even small coordinated gene expression changes in a pathway can lead to a greater biological effect. This identified COVID-19 and cytokine-cytokine receptor interaction pathways amongst the top enriched pathways. Thus, transcriptome profiling suggests that vaccination prevented inflammatory responses after the challenge.

### B. Materials and Methods

Viruses. The SARS-CoV-2 strain used for animal challenge is the first US isolate SARS-CoV-2 USA_WA1/2020 from the Washington State patient identified on Jan. 22, 2020 (GenBank accession number: MN985325 (SEQ ID NO:1)) (Harcourt et al., 2020). Passage 3 was obtained from the World Reference Center for Emerging Viruses and Arboviruses (WRCEVA) at UTMB and underwent two more passages on Vero-E6 cells (passage 5). For virus neutralization assay, viral stock which underwent two additional passages, was used (passage 7). Alpha (lineage B.1.1.7, isolate SARS-CoV-2/human/USA/CA_CDC_5574/2020), Beta (lineage B.1.351, isolate hCoV-19/USA/MD-HP01542/2021) and Gamma (lineage P.1, isolate SARS-CoV-2/human/USA/MD-MDH-0841/2021; GenBank accession number: MW621433) variants of SARS-CoV-2 were obtained from WRCEVA, passage 2 (Gamma) or 3 (Alpha, Beta), and subjected to an additional passage on Vero-E6 cells. The inoculum stocks used in virus neutralization assay were passages 3 or 4, respectively. The recombinant SARS-CoV-2 expressing mNeonGreen reporter-WA1/2020 (Xie et al., 2020), Kappa (lineage B.1.617.1) and Delta (lineage B.1.617.2) variants-were kindly provided by Dr. Pei-Yong Shi (UTMB). For all SARS-CoV-2 variants, S ORF was sequenced in viral stocks used for in vitro experiments. The following mutations were identified: Gamma, K417T; Delta, deletion of amino acids 689-691. No other changes were found compared to the corresponding reference sequences. All studies involving infectious SARS-CoV-2 were performed under BSL-3 containment of the Galveston National Laboratory (GNL), UTMB. HPIV3 wild-type (wt), strain JS (Bukreyev et al., 2006b) was used as vehicle control in vaccination experiments.

Generation of vaccine constructs. HPIV3-vectored vaccine constructs expressing SARS-CoV-2 full S protein (1-1273 aa), its S1 subunit (1-685 aa), RBD1 (319-591 aa) or RBD2 (319-529 aa) were generated (the amino acid sequences of S protein are based on the first U.S. SARS-CoV-2 isolate, GenBank accession no. MN985325 (SEQ ID NO:1). The vaccine vector was based on HPIV3 SUDV GP full-length clone (Kimble et al., 2019) generated with HPIV3 reverse genetics system (Durbin et al., 1997) kindly provided by Peter L. Collins (NIAID, NIH). For optimal immunogen expression in mammalian cells, different strategies for codon optimization in S ORF were utilized, resulting in the two sets of constructs. Coding sequences for full S, S1, RBD1 or RBD2 flanked by NcoI and BlpI restriction sites were PCR-amplified with PfuUltra high-fidelity DNA polymerase (Agilent Technologies) using cDNA not codon-optimized for eucaryotic translation (set “a”) or codon-optimized (set “b”). The codon-optimized (Genscript) cDNA in pCR-Blunt II-TOPO vector was kindly provided by Dr. Matthias J. Schnell (Thomas Jefferson University). Because of the overlapping NcoI site with the ORF start (ATGG), the second amino acid in full S and S1 constructs was different from that in an original SARS-CoV-2 S sequence (P→V). For RBD constructs, Val codon (GTC or GTT) was introduced immediately downstream of the start codon, followed by sequences encoding for 319-591 aa or 319-529 aa. The reverse primers were designed to introduce stop-codons (TGA for HPIV3/full S_b, and TAA for other 7 constructs) followed by four-nucleotide TATA sequence to comply with the “rule of six” for paramyxovirus genomes (Kolakofsky et al., 1998). Obtained PCR fragments were introduced into HPIV3 SUDV GP by NcoI and BlpI restriction sites to replace SUDV GP ORF. A complete nucleotide sequences of the resulting full-length clones were verified by Sanger sequencing. The full-length clones were next used to recover vaccine constructs as previously described (Kimble et al., 2019). Passage 0 aliquots were amplified in LLC-MK2 cells for 2-3 passages to raise the sufficient stocks for experiments. The inserts encoding for S protein or its fragments were sequenced in genomic RNA of all recovered viruses, with no mutations identified. To determine viral titers, viruses were inoculated onto LLC-MK2 monolayers, and incubated for 8 days under 0.45% methylcellulose overlay. Then, monolayers were fixed with ice-cold methanol, and viral plaques were immunostained with wiffleball fluid of HPIV3-infected rabbits provided by Drs. Peter Collins and Ursula Buchholz (NIAID) and HRP-labeled goat anti-rabbit IgG secondary antibody (ThermoFisher Scientific).

Western blot. The expression of SARS-CoV-2 S proteins by infected cells was detected by Western blot as in (Kimble et al., 2019). Briefly, monolayers of LLC-MK2 cells were infected with HPIV3/full S_a, HPIV3/full S_b, HPIV3/S1_a, HPIV3/S1_b, HPIV3/RBD1_a, HPIV3/RBD1_b, HPIV3/RBD2_a, HPIV3/RBD2_b, HPIV3-wt, or mock-infected. After 48 hours, cells were lysed and run on a 4 to 12% SDS-PAGE gel. The recombinant S protein (Sino Biological) was loaded at 2.5 μg as control. The membrane was stained with primary rabbit anti-SARS-CoV-2 S polyclonal (Sino Biological) and mouse actin pan monoclonal (ThermoFisher Scientific) antibodies, followed by secondary goat anti-rabbit IgG 800 CW and anti-mouse IgG 680 RD antibodies (LI-COR). Protein bands were visualized with a LI-COR Odyssey Fc imaging system.

Hamster studies. Six- to seven-week-old female golden Syrian hamsters were obtained from Envigo. Hamsters were housed at 4 animals per isolator cage and provided food and water ad libitum. SARS-CoV-2 challenge study was conducted in an animal biosafety level 3 (ABSL-3) GNL facility. The animal protocol for testing of HPIV3-based vaccine constructs in hamsters was approved by the UTMB Institutional Animal Care and Use Committee (IACUC) in compliance with the Animal Welfare Act and other applicable federal statutes and regulations relating to animals and experiments involving animals. Five groups of hamsters (n=10 per group) were intranasally vaccinated with HPIV3/full S, HPIV3/S1, HPIV3/RBD1, HPIV3/RBD2, or mock-vaccinated with HPIV3-wt, at 10⁶ PFU in a total volume of 100 μl (approximately 50 μl into each nostril). Four weeks after the vaccination, animals were challenged with SARS-CoV-2 intranasally at 10⁵ PFU in a total volume of 100 μl administered as described above. Over the infection course, hamsters were monitored daily for weight changes. On each serial endpoint day (days 3 and 14 post-challenge), lungs, nasal turbinates and sera were collected from 5 hamsters per group. Additionally, serum samples were collected from all animals at one day prior to vaccination, and one day prior to challenge. One animal in HPIV3-wt group was found dead on day 15 after vaccination (13 days prior to challenge) with no clinical signs of disease and was therefore excluded from the subsequent analysis.

In a separate study conducted under ABSL-2 containment, four groups of hamsters (n=5 per group) were intranasally vaccinated with HPIV3/full S, HPIV3/S1, HPIV3/RBD1, or mock-vaccinated with HPIV3-wt, at 1×10⁶ PFU in a total volume of 100 μl. Four weeks after the vaccination, animals were euthanized, lungs and spleen were collected, and subjected to an analysis for the specific immune cell populations by flow cytometry.

Analysis of viremia. The right lung and nasal turbinates were homogenized in Leibovitz L-15 cell culture medium supplemented with 10% fetal bovine serum (FBS) and 1% Antibiotic-Antimycotic (ThermoFisher Scientific) using the TissueLyser II bead mill and 5 mm stainless steel beads (Qiagen) and briefly centrifuged (5 min, 2000×g, 4° C.). The duplicate 10-fold serial dilutions of homogenates were prepared in MEM medium and adsorbed on Vero-E6 monolayers in 96-well plates for 1 hour at 37° C., 5% CO₂. The virus inoculum was then replaced with 0.55% methylcellulose overlay. After 2 days, an overlay was removed, and cells were fixed with 10% formalin at 4° C. for 24 hours. Fixed monolayers were stained with 10% formalin containing 0.25% crystal violet (ThermoFisher Scientific) at room temperature for 20 min and washed with water. Plaques were counted, and virus load per gram tissue was determined.

RNA isolation. The aliquots of supernatants from lung and nasal turbinate homogenates were dissolved in TRIzol LS (Life Technologies). Total RNA was isolated using Direct-zol™ RNA Microprep kit (Zymo Research) with on-column DNAse digestion according to the manufacturer's recommendations. The final RNA solutions were stored at −80° C. until used for qRT-PCR or transcriptome analysis.

Analysis of viral load by qRT-PCR. Replicating viral RNA was determined in the lungs and nasal turbinates by measuring subgenomic SARS-CoV-2 E gene RNA by qRT-PCR in triplicates as described previously (Meyer et al., 2021). An Ultramer DNA oligo (IDT Bioservices) spanning the amplicon was used to generate standard curves to calculate the sgRNA copies per microgram of the total RNA.

Total IgG ELISA. The 96-well microplates (Greiner Bio-One) were coated with 1 μg/mL SARS-CoV-2 S protein (Sino Biological) diluted in phosphate buffered saline (PBS). After overnight incubation at 4° C., plates were washed 4 times with PBS with 0.05% Tween-20 and blocked with SuperBlock blocking buffer (ThermoFisher Scientific) for 2 hours at 37° C. After washing, the duplicate 5-fold serial dilutions of hamster serum, starting at 1:10, were added in 50 μl/well (assay diluent: PBS+0.05% Tween-20+5% goat serum). Plates were incubated for 2 hours at 37° C. and washed 4 times, and then HRP-conjugated goat anti-Syrian hamster IgG (Abcam) was added at 1:10,000 dilution in assay diluent. Plates were incubated for 1 hour at 37° C. and washed 4 times, and then one-component SureBlue reserve TMB microwell peroxidase substrate (Sera Care) was used to detect bound antibody. After incubation at room temperature for 10 min, the reaction was stopped by adding TMB BlueSTOP solution (Sera Care), and the absorbance was measured at optical density (OD) 630 nm. Titers were determined using a four-parameter logistic curve fit in GraphPad Prism 6.07 software and defined as the reciprocal dilution at approximately OD_{630 nm}=0.075 (normalized to the pre-vaccination serum sample for each animal).

Virus neutralization assays. A total of 200 PFU of mNeonGreen SARS-CoV-2 (WA1/2020, Kappa variant, or Delta variant) were incubated in duplicates with 2-fold serial dilutions of serum starting from the initial dilution of 1:20 for 1 hour at 37° C. in MEM medium containing 2% FBS and 0.1% gentamicin sulfate. Virus-serum mixtures were then added to Vero-E6 monolayers in black polystyrene 96-well plates with clear bottoms and incubated at 37° C., 5% CO₂. Plates were read using the Cytation 7 Cell Imaging Multi-Mode Reader (BioTek) (EX 488 nm, EM 528 nm) at 48 hours post-infection. The percentage of neutralization was determined by the following formula: 100−((X−Z)*100/(Y−Z)), where X represents MFI readout of sample monolayers, Y-MFI of virus-infected monolayers with no serum added, and Z-MFI of uninfected monolayers (background).

The selected serum samples were also tested in a standard plaque reduction neutralization assay against biological isolates of the WA1/2020 strain and Alpha, Beta and Gamma VOCs. A total of 100 PFU of SARS-CoV-2 were incubated in duplicates with 2-fold serial dilutions of serum starting from the initial dilution of 1:20 for 1 hour at 37° C. in MEM medium containing 2% FBS and 0.1% gentamicin sulfate. Virus-serum mixtures were then added to Vero-E6 monolayers in 24-well plates and incubated for 1 hour at 37° C., 5% CO₂. The virus inoculum was then replaced with 0.67% methylcellulose overlay. After 3 days, an overlay was removed, and cells were fixed with 10% formalin at 4° C. for 24 hours. Fixed monolayers were stained with 10% formalin containing 0.25% crystal violet at room temperature for 20 min and washed with water. In all neutralization assays, IC60 values were calculated in GraphPad Prism 6.07 software.

Peptide microarray. Glass slides with imprinted 316 overlapping 15 residue peptides corresponding to SARS-CoV-2 S sequence (protein ID: PODTC2) were produced by JPT Peptide Technologies GmbH (Berlin, Germany). The first peptide corresponds to residues 1 to 15 and each successive peptide begins 4 residues downstream (5 to 19, 9 to 23, etc.). Post-challenge sera were virus-inactivated by γ-irradiation at 5 MRad prior to the analysis. Slides were incubated with serum samples diluted at 1:50 for 1 h at 30° C. followed by 5 washes in JPT washing buffer. Then, slides were incubated with 0.1 μg/mL Cy5-conjugated goat anti-Syrian hamster IgG secondary antibody (Abcam) followed by 5 washes in JPT washing buffer. After additional 2 washes in deionized water, the slides were dried by centrifugation. MFI was recorded for each spot on the GenePix 4000b at 650 V and analyzed by GenePix Pro 7. Post-vaccination serum samples from animal group treated with HPIV3/full S vaccine were analyzed in triplicates; for all other samples, single runs were performed. Pre-vaccination sera served as background control. The results were expressed as mean values of specific signals produced by post-vaccination sera minus background, for each individual peptide.

Histopathology. Following euthanasia of hamsters with ketamine/xylazine injection, necropsy was performed, and lungs were inspected for gross lesions. A representative specimen of lungs (right lower lobe) was collected in 10% buffered formalin for histological examination. Formalin-fixed tissues were processed per a standard protocol for histological analysis, and 4 μm-thick sections were cut and stained with hematoxylin and eosin (HE). Lung sections were examined under light microscopy using an Olympus CX43 microscope for the extent of inflammation, size of inflammatory foci, and changes in alveoli, alveolar septa, airways, and blood vessels. The slides were imaged in a digital scanner (Leica Aperio LV1). The blinded tissue sections were semi-quantitatively scored for pathological lesions using the criteria described in Table 3.

TABLE 3
Criteria for histopathology scoring.

| | Scores→ | 0 | 1 | 2 | 3 | 4 |
|---|---|---|---|---|---|---|
| A | Extent of | 0 | <10 | 10-30 | 30-60 | >60 |
| | inflammation | | | | | |
| | (% tissue | | | | | |
| | involved) | | | | | |
| B | Inflammatory | No | Patchy | Patchy | Large | Large |
| | foci type | inflammation | inflammatory | inflammatory | inflammatory | inflammatory |
| | | | foci, | foci, many | foci, few | foci, many |
| | | | few (<2) | (>2) | (<2) | (>2) |
| C | Alveolar | Thin and | Thickened | Thickened | Thickened | Thickened |
| | septa | delicate | in <10% HPF | in <30% HPF | in <60% HPF | in >60% HPF |
| D | Airways | Clear; no | Few cells in | Moderate | More cells in | Occlusion of |
| | | cells | airway | cells in | air way; | air |
| | | | | airway | Epithelial | way/epithelial |
| | | | | | hyperplasia | hyperplasia |
| | | | | | | or desquamation |
| E | Alveoli/ | Clear; no | Few cells. Few | Moderate | More | Abundant |
| | perivascular | inflammatory | PMN or MNC | cells/ | cells/PVC/ | cells/large |
| | cuff/blood | cells | | PVC/mild | more | PVC/severe |
| | vessels/ | | | congestion/ | congestion | congestion or |
| | pleuritis/cell | | | mild | and | pleuritis/ |
| | types | | | pleuritis/ | pleuritis/ | mixed cells |
| | | | | mostly MNC | more MNC and | |
| | | | | | PMN | |

The criteria were adapted from (Matute-Bello et al., 2011). HPF—high power field (>10x); PMN—polymorphonuclear cells/heterophils; MNC—mononuclear cells including lymphocytes and macrophages; PVC—perivascular cuff.

Immunohistochemistry. SARS-CoV-2 antigen (NP) was identified in situ on formalin-fixed paraffin-embedded tissue sections by immunohistochemical (IHC) staining. Briefly, paraffin-embedded lung specimens were serially sectioned (5 μm). A citric acid-based antigen unmasking solution (Vector Laboratories Inc.), pH=6 was used for the 20 min antigen retrieval process using a microwave (BioTek EZ-Retriever, BioGenix) heat treatment. A primary antibody specific for SARS-CoV-2 nucleocapsid (GeneTex) followed by a secondary anti-rabbit IgG AP (Vector Laboratories Inc.) were used for the staining. Viral antigen was visualized with the Vector® Red AP Substrate Kit (Vector Laboratories Inc.) and analyzed by light microscopy.

Cytokine ELISA. The level of cytokines in culture supernatants or lung homogenates were quantified using Immunotag® Hamster ELISA kits (G-Biosciences), following the manufacturer's instructions. Briefly, 100 μl of the lung homogenates or culture supernatants (4-fold diluted in Assay diluent) were incubated in duplicate wells pre-coated with cytokine-specific capture antibodies for 90 min at 37° C. After two washes, 100 μl of biotin-labeled detection antibodies were added for 60 min at 37° C. HRP-streptavidin conjugate was used for immunodetection with TMB substrate. The absorbance at 450 nm was measured. A standard curve obtained for each cytokine with known standards was used for calculating the level of cytokines.

Immunophenotyping of lung and spleen cells by flow cytometry. For immunophenotyping, single cell suspensions were prepared from the lungs and spleens of vaccinated and control hamsters. Briefly, the lungs were cut into small pieces and suspended in 1 ml digestion buffer containing 3 mg/ml collagenase type II and 40 U/ml rDNase I (Worthington Biochemical). Following digestion for 40 min at 37° C., the lung cells were passed through 70 μm cell strainer and collected as pellet by centrifugation (250×g, 10 min). Red blood cells were lysed with ACK lysis buffer (Lonza) and washed out with an excess of 0.5% BSA in 1×PBS (PBS/BSA). Cells were further processed for gradient centrifugation using Histopaque-1077 (Sigma-Aldrich) to enrich immune cells, following standard procedure. The final cell pellet was resuspended in PBS/BSA and cells were counted in TC20™ Automated Cell Counter (Bio-Rad Laboratories). The spleen samples were gently minced with syringe plunger on top of the cell strainer and red blood cells were lysed as above. The cells were resuspended in PBS/BSA buffer and counted.

The supply of antibodies specific to hamster immune cells and proteins is very limited. As such, cell staining using anti-mouse antibodies was optimized. A previous study has shown that several anti-mouse antibodies bind to hamster immune cells well (Kaewraemruaen et al., 2016). Hamster lung and spleen cells were incubated with Live/Dead Fixable Aqua stain (BioLegend) and monoclonal antibodies specific for mouse CD4-FITC (clone GK1.5), CD8-PE (clone eBio341, Invitrogen), CD44-Pacific Blue (clone IM7), CD62L-APC/Fire (clone MEL-14), and CD25 PE/cy7 (clone PC61) T cell markers for 30 min at 4° C. in the dark. As well, cells were stained separately with B220-BV421 (clone RA3-6B2) and CD19-PerCP (clone 4G7) for B cell markers. All these antibodies, except CD8-PE, were purchased from the BioLegend. The stained cells were washed thoroughly with PBS/BSA and fixed in 2% paraformaldehyde (PFA) overnight. Fixed cells were analyzed on an LSR Fortessa flow cytometer (BD Biosciences) and frequencies of each cell type were calculated using FlowJo software (v10.8.0_CL).

Cell culture, peptide treatment, flow cytometry and cytokine quantification. To test in vitro recall response of vaccine candidates, the lung and spleen immune cells were cultured in 96-well plates (5×10⁵ cells/well) using RPMI-10% FBS (Gibco) supplemented with 1% Penicillin-Streptomycin-Glutamine (Sigma-Aldrich). The cells were treated with peptides of SARS-CoV-2 spike protein (PepTivator peptides Prot_S and Prot_S1) obtained from Miltenyi Biotec. As per manufacturer, the average purity of peptides was >70% (HPLC). The peptides were dissolved per manufacturer's directions in sterile water and used at a concentration of 1 mg/ml. The cells were stimulated with peptides for 14 h at 37° C./5% CO₂, and Brefeldin A (1 mg/ml, Sigma-Aldrich) was added for 2 h prior to collection of cells and culture supernatants. As a positive control, cells were stimulated with PMA/Ionomycin (1 mg/ml, Sigma-Aldrich) and, as a negative control, cells were left untreated.

After peptide treatment, the cells were collected and centrifuged at 200×g for 5 min. The culture supernatants were harvested and stored frozen at −80° C. for ELISA quantification of cytokines as described below. The cells were surface stained with CD4-FITC (clone GK1.5) and CD8-PE (clone eBio341) antibodies for 30 min. After washing with PBS/BSA, cells were fixed with 2% PFA, permeabilized using Intracellular Fixation & Permeabilization Buffer (BioLegend) and stained with IFNγ-APC (clone XMG1.2, BioLegend) for 30 min at 4° C. in the dark. Then, cells were washed, resuspended in PBS, and analyzed on an LSR Fortessa flow cytometer (BD Biosciences). The frequency of cell types and IFNγ expressions were calculated using FlowJo software (v10.8.0_CL).

Library construction, high-throughput sequencing and transcriptome analysis. Libraries for deep sequencing were made using the Smart-3SEQ protocol (Foley et al., 2019). Briefly, the first strand primer was annealed to 1 μl of sample RNA and extended with SMARTScribe reverse transcriptase (Clontech, Inc). Second strand synthesis was performed after the addition of the second strand primer, and adapter sequences with unique indexes were added with 15 cycles of PCR with NEBNext single index adapters (New England BioLabs). PCR products were purified with AMPure XP SPRI beads (Beckman Coulter Life Sciences), quantified, and pooled for sequencing on an Illumina NextSeq 550 High Output Flow Cell with the single-end 75 base protocol.

The Smart-3SEQ protocol adds a 5 base unique molecular identifier (UMI) and 3 Gs to the 5′ end of each sequence. These were removed from the reads and the UMI was added to the read name with the umi_homopolymer.py software provided by the Smart-3SEQ authors. Reads were aligned to the Mesocricetus auratus NCBI assembly GCF_000349665.1 using STAR version 2.7.5c (Dobin et al., 2013) using the parameters recommended by the software authors for the Encode consortium. FeatureCounts software (Liao et al., 2014) was used to count reads per gene using the NCBI annotation release 102. The counttable was used as an input into DESeq2 (Love et al., 2014), and differential gene expression was estimated following the DESeq2 vignette provided with the software. Hierarchical clustering of the genes was done with heatmap program in R. Gene ontology enrichment analysis was performed with DAVID v6.8 (Huang da et al., 2009a, b). Gene set enrichment analysis was performed with the GAGE software (Luo et al., 2009) and Pathview software (Luo and Brouwer, 2013) was used to produce the KEGG pathway figures, both following the vignettes provided by the authors.

Hamster specific gene lists for stromal and immune lung cells were taken from a published single cell study of hamster SARS-CoV-2 infection (Meyer et al., 2021). These gene sets were used to compute ssGSEA scores from log transformed and quantile normalized bulk RNA seq data using the “gsva” function with parameters method=“ssgsea” and ssgsea.norm=T.

Statistical analysis. Statistical analyses and generation of graphs were performed using GraphPad Prism version 6.07 (GraphPad Software). One- or two-way ANOVA with multiple comparisons (Fisher's LSD test) or a T-test were used for determination of statistical significance.

### REFERENCES

COVID-19 (coronavirus) in babies and children (Mayo Clinic). (2021) URL www.mayoclinic.org/diseases-conditions/coronavirus/in-depth/coronavirus-in-babies-and-children/art-20484405
An et al., (2021). Science advances 7.
Andreano and Rappuoli, (2021). Nature medicine 27, 759-761.
Baden et al., (2021). The New England journal of medicine 384, 403-416.
Bolles et al., (2011). J Virol 85, 12201-12215.
Bricker et al., (2021). Cell reports 36, 109400.
Bridges et al., (2021). Thorax.
Bukreyev et al., (2006a). Journal of virology 80, 10293-10306.
Bukreyev et al., (2006b). Journal of virology 80, 2267-2279.
Bukreyev et al., (2010). Virology 399, 290-298.
Byrne et al., (2015). Thorax 70, 1189-1196.
Clements et al., (1991). J Clin Microbiol 29, 1175-1182.
Corti et al., (2016). Science (New York, NY) 351, 1339-1342.
Dai et al., (2020). Cell 182, 722-733.e711.
Dobin et al., (2013). Bioinformatics (Oxford, England) 29, 15-21.
Durbin et al., (1997). Virology 235, 232-232.
Feng et al., (2020). Nature communications 11, 4207.
Foley et al., (2019). Genome research 29, 1816-1825.
Gao et al., (2020). Science (New York, NY) 369, 77-81.
Harcourt et al., (2020). Emerg Infect Dis 26, 1266-1273.
Hassan et al., (2021a). Cell reports Medicine 2, 100230.
Hassan et al., (2020). Cell 183, 169-184.e113.
Hassan et al., (2021b). Cell reports 36, 109452.
Hennrich et al., (2021). PLOS pathogens 17, e1009064.
Hoffmann et al., (2020). Cell 181, 271-280.e278.
Honda-Okubo et al., (2015). J Virol 89, 2995-3007.
Huang et al., (2020). Lancet (London, England) 395, 497-506.
Huang da et al., (2009a). Nucleic Acids Res 37, 1-13.
Huang da et al., (2009b). Nat Protoc 4, 44-57.
Ilinykh et al., (2018). PLOS pathogens 14, e1007204.
Imai et al., (2020). PNAS of the United States of America 117, 16587-16595.
Jiang et al., (2020). Nature communications 11, 3581.
Kaewraemruaen et al., (2016). Parasite immunology 38, 688-697.
Keech et al., (2020). The New England journal of medicine 383, 2320-2332.
Khoury et al., (2021). Nature medicine 27, 1205-1211.
Kim et al., (2020). Cell host & microbe 27, 704-709.e702.
Kimble et al., (2019). Journal of virology 93.
King et al., (2019). Current opinion in virology 34, 140-148.
Kolakofsky et al., (1998). Journal of virology 72, 891-899.
Konings et al., (2021). Nature microbiology 6, 821-823.
Krause et al., (2021). The New England journal of medicine 385, 179-186.
Ku et al., (2021). Cell host & microbe 29, 236-249.e236.
Kurup et al., (2021). PLOS pathogens 17, e1009383.
Lau et al., (2021). Nature communications 12, 63.
Lee and Lowen, (2021). Current opinion in virology 48, 73-81.
Li et al., (2021). Cell reports 34, 108915.
Liao et al., (2014). Bioinformatics (Oxford, England) 30, 923-930.
Liu et al., (2020). Nature 584, 450-456.
Liu et al., (2004). Lancet (London, England) 363, 938-947.
Logunov et al., (2021). Lancet (London, England) 397, 671-681.
Love et al., (2014). Genome biology 15, 550.
Lu et al., (2021). PNAS of the United States of America 118.
Luo and Brouwer, (2013). Bioinformatics 29, 1830-1831.
Luo et al., (2009). BMC bioinformatics 10, 161.
Lycke, (2012). Nature reviews Immunology 12, 592-605.
Malherbe et al., (2021). NPJ vaccines 6, 91.
Mao et al., (2019). Nature methods 16, 607-610.
McMahan et al., (2021). Nature 590, 630-634.
Mercado et al., (2020). Nature 586, 583-588.
Meyer et al., (2021). J Clin Invest 131.
Misasi et al., (2016). Science (New York, NY) 351, 1343-1346.
Mosser and Edwards, (2008). Nature reviews Immunology 8, 958-969.
Murphy and Weaver, (2017). Janeway's immunobiology, 9th edn (New York: Garland Science).
Ni et al., (2020). Immunity 52, 971-977.e973.
Piccoli et al., (2020). Cell 183, 1024-1042.e1021.
Poh et al., (2020). Nature communications 11, 2806.
Polack et al., (2020). The New England journal of medicine 383, 2603-2615.
Premkumar et al., (2020). Science immunology 5.
Robbiani et al., (2020). Nature 584, 437-442.
Routhu et al., (2021). Nature communications 12, 3587.
Seydoux et al., (2020). Immunity 53, 98-105.e105.
Shang et al., (2020). Nature 581, 221-224.
Smith et al., (2020). Nature communications 11, 2601.
Stevens et al., (2021). Curr Opin Virol 50, 49-58.
Subbarao, (2021). Cell host & microbe 29, 1111-1123.
Tseng et al., (2012). PLOS One 7, e35421.
van Doremalen et al., (2020). Nature 586, 578-582.
Voysey et al., (2021). Lancet (London, England) 397, 881-891.
Walls et al., (2020). Cell 181, 281-292.e286.
Wang et al., (2020). Cell 182, 713-721.e719.
Watanabe et al., (2020). Science (New York, NY) 369, 330-333.
Wrapp et al., (2020). Science (New York, NY) 367, 1260-1263.
Wu et al., (2020). Nature communications 11, 4081.
Xie et al., (2020). Cell host & microbe 27, 841-848.e843.
Xiong et al., (2020). Nat Struct Mol Biol 27, 934-941.
Yang et al., (2008). Virology 377, 255-264.
Yu et al., (2020). Science (New York, NY) 369, 806-811.
Zhang et al., (2020). Nature 583, 437-440.
Zhou et al., (2021). Cell host & microbe 29, 551-563.e555.
Zhu et al., (2020). The New England journal of medicine 382, 727-733.

1. A SARS-CoV-2 vaccine comprising an engineered Human parainfluenza virus (HPIV) encoding a SARS-CoV-2 spike protein (S protein) in a transcriptional cassette.

2. The vaccine of claim 1, wherein the Human parainfluenza virus is a Human parainfluenza virus type 3 (HIPV3).

3. The vaccine of claim 1, wherein the SARS-CoV-2 S protein peptide is a SARS-CoV-2 S protein.

4. The vaccine of claim 1, wherein the SARS-CoV-2 spike peptide is a SARS-CoV-2 S protein S1 subunit.

5. The vaccine of claim 4, wherein the SARS-CoV-2 spike peptide is a SARS-CoV-2 S protein receptor binding domain (RBD).

6. The vaccine of claim 1, wherein the transcriptional cassette comprises a HPIV3-specific gene-start and gene-end transcriptional signals upstream and downstream of the SARS-CoV-2 S protein encoding region.

7. The vaccine of claim 1, wherein the SARS-CoV-2 S protein transcriptional cassette is incorporated in the P-M intergenic sequence of the engineered HPIV.

8. A human parainfluenza virus (HPIV)/SARS-CoV-2 construction comprising a HPIV genome having a SARS-CoV-2 transcription cassette, wherein the transcription cassette encodes for all or part of a SARS-CoV-2 spike protein.

9. The construct of claim 8, wherein the transcriptional cassette comprises a HPIV3-specific gene-start and gene-end transcriptional signals upstream and downstream, respectively, of the SARS-CoV-2 S protein encoding region.

10. The construct of claim 8, wherein the SARS-CoV-2 S protein transcriptional cassette is incorporated in the P-M intergenic sequence of the engineered HPIV.

11. The construct of claim 8, wherein the construct has a nucleotide sequence that is at least 80, 90, 95, 98, or 100% identical to SEQ ID NO: SEQ ID NO:3, SEQ ID NO: 4, SEQ ID NO:5, SEQ ID NO:6, SEQ ID NO:7, SEQ ID NO:8, SEQ ID NO:9, or SEQ ID NO:10.

12. The construct of claim 8, wherein the construct has a nucleotide sequence that is at least 98% identical to SEQ ID NO: SEQ ID NO:3, SEQ ID NO:4, SEQ ID NO: 5, SEQ ID NO:6, SEQ ID NO:7, SEQ ID NO:8, SEQ ID NO:9, or SEQ ID NO: 10.

13. The construct of claim 8, wherein the construct has a nucleotide sequence of SEQ ID NO: SEQ ID NO:3, SEQ ID NO:4, SEQ ID NO:5, SEQ ID NO:6, SEQ ID NO: 7, SEQ ID NO:8, SEQ ID NO:9, or SEQ ID NO:10.

14. The construct of claim 8, where in the construct is an RNA.

15. A DNA construct encoding the construct of claim 8.

16. A recombinant human parainfluenza virus containing the construct of claim 8.

17. A method of inducing an antigen-specific immune response in a subject, the method comprising administering via intranasal administration to the subject the vaccine of claim 1 to produce an antigen-specific immune response in the subject.

18. The method of claim 17 wherein the vaccine composition comprises 10³ to 10⁸ pfu of a vaccine vector.

19. A composition comprising a vaccine of claim 1 formulated for intranasal administration.
